SEQUENCE LISTING (33 sequences)

SEQ ID NO: 1 - protein, 27 aa, Artificial Sequence
  Peptide fragment from alphacoronavirus RNA dependent RNA polymerase (RdRp)
TKRNVLPTMTQLNLKYAISGKERARTV

SEQ ID NO: 2 - protein, 27 aa, Artificial Sequence
  Peptide fragment from betacoronavirus RNA dependent RNA polymerase (RdRp)
TKRNVIPTITQMNLKYAISAKNRARTV

SEQ ID NO: 3 - protein, 27 aa, Artificial Sequence
  Peptide fragment from betacoronavirus RNA dependent RNA polymerase (RdRp)
TKRNVLPTLTQMNLKYAISAKNRARTV

SEQ ID NO: 4 - protein, 19 aa, Artificial Sequence
  Peptide fragment from alphacoronavirus membrane protein of protein M (MP)
PTGVTLTLLSGNLYAEGFK

SEQ ID NO: 5 - protein, 19 aa, Artificial Sequence
  Peptide fragment from alphacoronavirus membrane protein of protein M (MP)
PTGVTLTLLSGTLLVEGYK

SEQ ID NO: 6 - protein, 20 aa, Artificial Sequence
  Peptide fragment from alphacoronavirus membrane protein of protein M (MP)
YPTGITVTLLSGVLYVDGHR

SEQ ID NO: 7 - protein, 19 aa, Artificial Sequence
  Peptide fragment from alphacoronavirus membrane protein of protein M (MP)
PTGVTLTLLSGVLLVDGHK

SEQ ID NO: 8 - protein, 21 aa, Artificial Sequence
  Peptide fragment from alphacoronavirus membrane protein of protein M (MP)
PTGITLTILSGTLFFDGIRIA

SEQ ID NO: 9 - protein, 23 aa, Artificial Sequence
  Peptide fragment from betacoronavirus membrane protein of protein M (MP)
YFIASFRLFARTRSMWSFNPETN

SEQ ID NO: 10 - protein, 23 aa, Artificial Sequence
  Peptide fragment from betacoronavirus membrane protein of protein M (MP)
YFVNSIRLFIRTGSWWSFNPETN

SEQ ID NO: 11 - protein, 24 aa, Artificial Sequence
  Peptide fragment from gammacoronavirus membrane protein of protein M (MP)
GYWIQSIRLFKRCRSWWSFNPESN

SEQ ID NO: 12 - protein, 18 aa, Artificial Sequence
  Peptide fragment from deltacoronavirus membrane protein of protein M (MP)
KLILLWLLQPFTLVVTIW

SEQ ID NO: 13 - protein, 419 aa, Homo sapiens
MAAPGSARRPLLLLLLLLLLGLMHCASAAMFMVKNGNGTACIMANFSAAFSVNYDTKSGPKNMTFDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSMSMLIPIAVGGALAGLVLIVLIAYLVGRKRSHAGYQTI

SEQ ID NO: 14 - protein, 410 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQDCSADDDNFLVPIAVGAALAGVLILVLLAYFIGLKHHHAGYEQF

SEQ ID NO: 15 - protein, 416 aa, Homo sapiens
MPRQLSAAAALFASLAVILHDGSQMRAKAFPETRDYSQPTAAATVQDIKKPVQQPAKQAPHQTLAARFMDGHITFQTAATVKIPTTTPATTKNTATTSPITYTLVTTQATPNNSHTAPPVTEVTVGPSLAPYSLPPTITPPAHTTGTSSSTVSHTTGNTTQPSNQTTLPATLSIALHKSTTGQKPVQPTHAPGTTAAAHNTTRTAAPASTVPGPTLAPQPSSVKTGIYQVLNGSRLCIKAEMGIQLIVQDKESVFSPRRYFNIDPNATQASGNCGTRKSNLLLNFQGGFVNLTFTKDEESYYISEVGAYLTVSDPETIYQGIKHAVVMFQTAVGHSFKCVSEQSLQLSAHLQVKTTDVQLQAFDFEDDHFGNVDECSSDYTIVLPVIGAIVVGLCLMGMGVYKIRLRCQSSGYQRI

SEQ ID NO: 16 - protein, 6 aa, Artificial Sequence
  Polyhistadine Tag
HHHHHH

SEQ ID NO: 17 - protein, 9 aa, Artificial Sequence
  Haemagglutinin (HA) tag
YPYDVPDYA

SEQ ID NO: 18 - protein, 3 aa, Artificial Sequence
  AAY peptide linker/spacer
AAY

SEQ ID NO: 19 - protein, 3 aa, Artificial Sequence
  GGS peptide linker/spacer
GGS

SEQ ID NO: 20 - protein, 5 aa, Artificial Sequence
  GPGPG peptide linker/spacer
GPGPG

SEQ ID NO: 21 - protein, 5 aa, Artificial Sequence
  EAAAK peptide linker/spacer
EAAAK

SEQ ID NO: 22 - protein, 8 aa, Artificial Sequence
  Core amino acid sequence motif of a 2A self- cleaving peptide
  VARIANT (2)..(2): X is any amino acid residue
  VARIANT (4)..(4): X is any amino acid residue
DXEXNPGP

SEQ ID NO: 23 - protein, 18 aa, Artificial Sequence
  T2A self-cleaving peptide
EGRGSLLTCGDVEENPGP

SEQ ID NO: 24 - protein, 19 aa, Artificial Sequence
  P2A self-cleaving peptide
ATNFSLLKQAGDVEENPGP

SEQ ID NO: 25 - protein, 20 aa, Artificial Sequence
  E2A self-cleaving peptide
QCTNYALLKLAGDVESNPGP

SEQ ID NO: 26 - protein, 22 aa, Artificial Sequence
  F2A self-cleaving peptide
VKQTLNFDLLKLAGDVESNPGP

SEQ ID NO: 27 - protein, 21 aa, Artificial Sequence
  T2A self-cleaving peptide with a GSG linker
GSGEGRGSLLTCGDVEENPGP

SEQ ID NO: 28 - protein, 22 aa, Artificial Sequence
  P2A self-cleaving peptide with GSG linker
GSGATNFSLLKQAGDVEENPGP

SEQ ID NO: 29 - protein, 23 aa, Artificial Sequence
  E2A self-cleaving peptide with GSG linker
GSGQCTNYALLKLAGDVESNPGP

SEQ ID NO: 30 - protein, 25 aa, Artificial Sequence
  F2A self-cleaving peptide with GSG linker
GSGVKQTLNFDLLKLAGDVESNPGP

SEQ ID NO: 31 - protein, 299 aa, Artificial Sequence
  CORV-1 polypeptide
TKRNVLPTMTQLNLKYAISGKERARTVAAYTKRNVIPTITQMNLKYAISAKNRARTVAAYTKRNVLPTLTQMNLKYAISAKNRARTVAAYPTGVTLTLLSGNLYAEGFKAAYPTGVTLTLLSGTLLVEGYKAAYPTGITVTLLSGVLYVDGHRAAYPTGVTLTLLSGVLLVDGHKAAYPTGITLTILSGTLFFDGIRIAAAYYFIASFRLFARTRSMWSFNPETNAAYYFVNSIRLFIRTGSWWSFNPETNAAYGYWIQSIRLFKRCRSWWSFNPESNAAYKLILLWLLQPFTLVVTIW

SEQ ID NO: 32 - protein, 308 aa, Artificial Sequence
  CORV-3 polypeptide
TKRNVLPTMTQLNLKYAISGKERARTVAAYTKRNVIPTITQMNLKYAISAKNRARTVAAYTKRNVLPTLTQMNLKYAISAKNRARTVAAYPTGVTLTLLSGNLYAEGFKAAYPTGVTLTLLSGTLLVEGYKAAYPTGITVTLLSGVLYVDGHRAAYPTGVTLTLLSGVLLVDGHKAAYPTGITLTILSGTLFFDGIRIAAAYYFIASFRLFARTRSMWSFNPETNAAYYFVNSIRLFIRTGSWWSFNPETNAAYGYWIQSIRLFKRCRSWWSFNPESNAAYKLILLWLLQPFTLVVTIWAAYHHHHHH

SEQ ID NO: 33 - protein, 1059 aa, Artificial Sequence
  CORV-2 polypeptide
TKRNVLPTMTQLNLKYAISGKERARTVAAYTKRNVIPTITQMNLKYAISAKNRARTVAAYTKRNVLPTLTQMNLKYAISAKNRARTVAAYPTGVTLTLLSGNLYAEGFKAAYPTGVTLTLLSGTLLVEGYKAAYPTGITVTLLSGVLYVDGHRAAYPTGVTLTLLSGVLLVDGHKAAYPTGITLTILSGTLFFDGIRIAAAYYFIASFRLFARTRSMWSFNPETNAAYYFVNSIRLFIRTGSWWSFNPETNAAYGYWIQSIRLFKRCRSWWSFNPESNAAYKLILLWLLQPFTLVVTIWAAYHHHHHHGSGEGRGSLLTCGDVEENPGPYPYDVPDYAMAAPGSARRPLLLLLLLLLLGLMHCASAAMFMVKNGNGTACIMANFSAAFSVNYDTKSGPKNMTFDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSMTKRNVLPTMTQLNLKYAISGKERARTVAAYTKRNVIPTITQMNLKYAISAKNRARTVAAYTKRNVLPTLTQMNLKYAISAKNRARTVAAYPTGVTLTLLSGNLYAEGFKAAYPTGVTLTLLSGTLLVEGYKAAYPTGITVTLLSGVLYVDGHRAAYPTGVTLTLLSGVLLVDGHKAAYPTGITLTILSGTLFFDGIRIAAAYYFIASFRLFARTRSMWSFNPETNAAYYFVNSIRLFIRTGSWWSFNPETNAAYGYWIQSIRLFKRCRSWWSFNPESNAAYKLILLWLLQPFTLVVTIWAAYSMLIPIAVGGALAGLVLIVLIAYLVGRKRSHAGYQTI

(19) United States
(12) Patent Application Publication
(10) Pub. No.: US 2022/0226465 A1
(43) Pub. Date: Jul. 21, 2022

(54) Coronavirus Immunogenic Compositions, Methods and Uses Thereof

(71) Applicant: ConserV Bioscience, Bicester (GB)

(72) Inventors: Olga PLEGUEZUELOS MATEO, Bicester (GB); Kimbell DUNCAN, Verbier (CH)

(73) Assignee: ConserV Bioscience, Bicester (GB)

(21) Appl. No.: 17/648,283

(22) Filed: Jan. 18, 2022

Related U.S. Application Data
(60) Provisional application No. 63/138,740, filed on Jan. 18, 2021.

(51) Int. Cl. A61K 39/215; A61P 43/00; A61P 37/02; A61K 48/00

(52) U.S. Cl. CPC A61K 39/215; A61P 43/00; A61K 38/00; A61K 48/0066; A61P 37/02

(57) ABSTRACT

The present specification discloses broad-spectrum, pan-Coronavirus immunogenic composition including amino acid-based, broad-spectrum, pan-Coronavirus immunogenic compositions and nucleotide-based, broad-spectrum, pan-Coronavirus immunogenic compositions, methods and uses for preventing or treating a Coronavirinae-based infection or pathology as well as methods and uses of creating, maintaining or restoring antigenic memory to one or more Coronavirus species, strains, genotypes, subgenotypes, serotypes or genetic/antigenic lineages in an individual or population of individuals.

This application claims the benefit of priority and is entitled to the filing date pursuant to 35 U.S.C. § 119(e) of U.S. Provisional Patent Application 63/138,740, filed Jan. 18, 2021, the content of which is hereby incorporated by reference in its entirety.

Severe acute respiratory syndrome coronavirus 1 (SARS-CoV-1) first emerged in China in 2002 and spread to other countries before brought under control. Currently, a new public health crisis is threatening the world, the emergence and spread of 2019 novel severe acute respiratory syndrome coronavirus 2 (SARS-CoV-2). This virus originated in Wuhan, Hubei province, China in December 2019 and on Jan. 30, 2020 the World Health Organization declared the outbreak as a Public Health Emergency of International Concern. The virus is typically transmitted by inhalation or contact with infected droplets and has an incubation period that ranges from 2 to 14 days. Symptoms are usually fever, persistent cough, loss of sense of smell and taste, sore throat, breathlessness, fatigue and malaise among others. The disease is mild in most people; in some (usually the elderly and those with comorbidities), it may progress to pneumonia, acute respiratory distress syndrome (ARDS), multi-organ dysfunction and death. Many people that contract this virus are asymptomatic but can spread the infection. The case fatality rate is estimated to range from 2% to 3%, although this is still debated. Infections are confirmed by detection of the virus in respiratory secretions (e.g. by a RT-PCR test) and exposure to the virus is confirmed by detection of IgG antibodies to the virus (serological testing), although some people are confirmed as infected by RT-PCR but do not seroconvert. Common laboratory findings include normal/low white cell counts with elevated levels of C-reactive protein (CRP). Computerized tomographic chest scans are usually abnormal even in those with no symptoms or mild disease. Anti-inflammatories such as dexamethasone and ventilation are the standard treatments for patients in intensive care. Prevention entails home isolation of suspected cases and those with mild illnesses and strict infection control measures at hospitals to prevent transmission through direct contact with the patient and droplets. This virus spreads faster than the other two epidemic/pandemic viruses in the family, the SARS-CoV-1 and Middle East respiratory syndrome coronavirus (MERS-CoV).

SARS-CoV-2 is a member of the family Coronaviridae and order Nidovirales. The family consists of two subfamilies, Coronavirinae and Torovirinae and members of the subfamily Coronavirinae are subdivided into four genera: (a) Alphacoronavirus contains the human coronavirus (HCoV)-229E and HCoV-NL63; (b) Betacoronavirus includes HCoV-0C43, Severe Acute Respiratory Syndrome coronavirus-1 (SARS-CoV-1), Severe Acute Respiratory Syndrome coronavirus-2 (SARS-CoV-2), HCoV-HKU1, and Middle Eastern respiratory syndrome coronavirus (MERS-CoV); (c) Gammacoronavirus includes mainly viruses that infect birds and; (d) Deltacoronavirus includes viruses isolated from pigs and birds.

Coronaviruses are enveloped positive sense RNA viruses ranging from 60 nm to 140 nm in diameter with spike like projections on its surface giving it a crown like appearance under the electron microscope: hence the name coronavirus. Four corona viruses namely HKU1, NL63, 229E and 0C43 have been in circulation in humans, and generally cause mild respiratory disease. There have been two events in the past two decades wherein crossover of animal Betacoronaviruses to humans has resulted in severe disease. The first such instance was in 2002-2003 when a new coronavirus of the β genera and with origin in bats crossed over to humans via the intermediary host of palm civet cats in the Guangdong province of China. This virus, designated as severe acute respiratory syndrome coronavirus, affected 8422 people mostly in China and Hong Kong and caused 916 deaths (mortality rate 11%) before being contained. Almost a decade later in 2012, the Middle East respiratory syndrome coronavirus (MERS-CoV), also of bat origin, emerged in Saudi Arabia with dromedary camels as the intermediate host and affected 2494 people and caused 858 deaths (fatality rate 34%).

SARS-CoV-2 is considered a novel human-infecting Betacoronavirus. Phylogenetic analysis of the SARS-CoV-2 genome indicates that the virus is closely related (with 88% identity) to two bat-derived SARS-like coronaviruses collected in 2018 in eastern China (bat-SL-CoVZC45 and bat-SL-CoVZXC21) and genetically distinct from SARS-CoV (with about 79% similarity) and MERS-CoV. Using the genome sequences of SARS-CoV-2, RaTG13, and SARS-CoV, a further study found that the virus is more related to BatCoV RaTG13, a bat coronavirus that was previously detected in Rhinolophus affinis from Yunnan Province, with 96.2% overall genome sequence identity. A study found that no evidence of recombination events detected in the genome of SARS-CoV-2 from other viruses originating from bats such as BatCoV RaTG13, SARS-CoV and SARSr-CoVs. Altogether, these findings may suggest that bats might be the original host of this virus.

Like most other members of the coronavirus family, Betacoronavirus exhibit high species specificity, but subtle genetic changes can significantly alter their tissue tropism, host range, and pathogenicity. A striking example of the adaptability of these viruses is the emergence of deadly zoonotic diseases in human history caused by SARS-CoV and MERS-CoV. In both viruses, bats served as the natural reservoir and humans were the terminal host, with the palm civet and dromedary camel the intermediary host for SARS-CoV and MERS-CoV, respectively. Intermediate hosts clearly play a critical role in cross species transmission as they can facilitate increased contact between a virus and a new host and enable further adaptation necessary for an effective replication in the new host. Because of the pandemic potential of SARS-CoV-2, careful surveillance is immensely important to monitor its future host adaptation, viral evolution, infectivity, transmissibility, and pathogenicity.

The host range of a virus is governed by multiple molecular interactions, including receptor interaction. The envelope spike (S) protein receptor binding domain of SARS-CoV-2 was shown structurally similar to that of SARS-CoV, despite amino acid variation at some key residues. Further extensive structural analysis strongly suggests that SARS-CoV-2 may use host receptor angiotensin-converting enzyme 2 (ACE2) to enter the cells, the same receptor facilitating SARS-CoV to infect the airway epithelium and alveolar type 2 (AT2) pneumocytes, pulmonary cells that synthesize pulmonary surfactant. In general, the spike protein of coronavirus is divided into the 51 and S2 domain, in which 51 is responsible for receptor binding and S2 domain is responsible for cell membrane fusion. The 51 domain of SARS-CoV and SARS-CoV-2 share around 50 conserved amino acids, whereas most of the bat-derived viruses showed more variation. In addition, identification of several key residues (Gln493 and Asn501) that govern the binding of SARS-CoV-2 receptor binding domain with ACE2 further support that SARS-CoV-2 has acquired the capacity for person-to-person transmission. Although, the spike protein sequence of receptor binding SARS-CoV-2 is more similar to that of SARS-CoV, at the whole genome level, SARS-CoV-2 is more closely related to bat-SL-CoVZC45 and bat-SL-CoVZXC21.

Similar to MERS-CoV and SARS-CoV-1, there is still no specific antiviral treatment for SARS-CoV-2. Drugs are being evaluated in line with past investigations into therapeutic treatments for SARS and MERS. Remdesivir which was developed for Ebola virus, has been approved to treat hospitaliaed severe cases of COVID-19 in the US. Neutralizing antibodies such as sotrovimab (Xevudy) and antiviral drugs such as molnupiravir (Lagevrio) have been approved in the United Kingdom as treatments for people with coronavirus (COVID-19) who are at highest risk of becoming seriously ill.

Accordingly, COVID-19 is clearly a serious disease of international concern. By some estimates it has a higher reproductive number than SARS-CoV-1, and more people have been reported to have been infected or died from it than SARS-CoV-1. Similar to SARS-CoV-1 and MERS-CoV, disrupting the chain of transmission is considered key to stopping the spread of disease. Several reviews of Coronavirus Disease (COVID-19) have been published, including Harapan, H. et al. Coronavirus disease 2019 (COVID-19): A literature review Journal of Infection and Public Health Volume 13, Issue 5, May 2020, Pages 667-673 and Singhal, T. A Review of Coronavirus Disease-2019 (COVID-19). Indian J Pediatr 87, 281-286 (2020), herein incorporated by reference.

Therefore, it is of great interest to develop new vaccines as well as new approaches to combating SARS-CoV-2 and associated disease and this and potential future variants of this highly zoonotic family of viruses. Provided herein are ribonucleic acid (RNA) vaccines that build on the knowledge that RNA (e.g., messenger RNA (mRNA)) can safely direct the body's cellular machinery to produce nearly any protein of interest, from native proteins to antibodies and other entirely novel protein constructs that can have therapeutic activity inside and outside of cells. A broad-spectrum, pan-Coronavirus immunogenic composition disclosed herein may be used to induce a balanced immune response against the current SARS-CoV-2 virus, and its potential future iterations, and other members of the Coronavirus family including the 4 genera: alpha, beta, gamma and deltacoronavirus.

### SUMMARY

Aspects of the present disclosure disclose a broad-spectrum pan-Coronavirus (COV) immunogenic composition. A broad-spectrum, pan-broad-spectrum, pan-COV immunogenic composition disclosed herein can be an amino acid-based immunogenic composition or a nucleotide-based immunogenic composition. A disclosed amino acid-based immunogenic composition comprises one or more COV peptide antigens. Examples of an amino acid-based immunogenic composition includes, without limitation, a peptide-based broad-spectrum, pan-broad-spectrum, pan-COV immunogenic composition and a recombinant protein-based broad-spectrum, pan-broad-spectrum, pan-COV immunogenic composition. A disclosed nucleotide-based immunogenic composition comprises a nucleotide sequence encoding one or more COV peptide antigens. A broad-spectrum, pan-broad-spectrum, pan-COV immunogenic composition disclosed herein may optionally also include one or more adjuvants. A broad-spectrum, pan-broad-spectrum, pan-COV immunogenic composition disclosed herein comprises one or more carriers and be formulated as a vaccine delivery system or a vaccine adjuvant-delivery system.

Other aspects of the present specification disclose methods of treating or preventing a Coronavirinae-based infection or pathology by administering a broad-spectrum, pan-broad-spectrum, pan-COV immunogenic composition disclosed herein to an individual. In addition, the present specification discloses a broad-spectrum, pan-broad-spectrum, pan-COV immunogenic composition disclosed herein for use in treating or preventing a Coronavirinae-based infection or pathology; use of a broad-spectrum, pan-broad-spectrum, pan-COV immunogenic composition disclosed herein in treating or preventing a Coronavirinae-based infection or pathology; and use of a broad-spectrum, pan-broad-spectrum, pan-COV immunogenic composition disclosed herein in the manufacture of a medicament for treating or preventing a Coronavirinae-based infection or pathology.

Other aspects of the present specification disclose methods of creating, maintaining, or restoring antigenic memory for one or more Coronavirus species, strains, genotypes, subgenotypes, serotypes or genetic/antigenic lineages in an individual or population of individuals. Such disclosed methods comprises administering a broad-spectrum, pan-broad-spectrum, pan-COV immunogenic composition disclosed herein to an individual or population of individuals. In addition, the present specification discloses a broad-spectrum, pan-broad-spectrum, pan-COV immunogenic composition disclosed herein for use in creating, maintaining or restoring antigenic memory for one or more Coronavirus species, strains, genotypes, subgenotypes, serotypes or genetic/antigenic lineages in an individual or population of individuals; use of a broad-spectrum, pan-broad-spectrum, pan-COV immunogenic composition disclosed herein in creating, maintaining or restoring antigenic memory to one or more Coronavirus species, strains, genotypes, subgenotypes, serotypes or genetic/antigenic lineages in an individual or population of individuals; and use of a broad-spectrum, pan-broad-spectrum, pan-COV immunogenic composition disclosed herein in the manufacture of a medicament for creating, maintaining or restoring antigenic memory for one or more Coronavirus species, strains, genotypes, subgenotypes, serotypes or genetic/antigenic lineages in an individual or population of individuals.

### DETAILED DESCRIPTION

Members of the family Coronaviridae are large, enveloped, positive-sense, single-stranded (ss) RNA viruses. They are the largest known RNA viruses, with genomes ranging from 25 kb to 32 kb and virions of 118 nm to 140 nm in diameter. The viral particles are large, roughly spherical particles with unique surface club- or petal-shaped surface projections of about 20 nm (the “peplomers” or “spikes”), which in electron micrographs of spherical particles create an image reminiscent of the solar corona.

The genome organization of Coronaviridae is 5′-methylated methylated-UTR-polycistron-3′UTR-polyadenylated (PolyA) tail. Two large, overlapping ORFs (ORF1a and ORF1b), which occupy the first two-thirds of the genome, encode the replicase polyprotein (pp1ab) which is initially expressed as a fusion protein but subsequently processed into two major non-structural proteins (pp1a and pp1ab) by a −1 programmed ribosomal frameshift. pp1a and pp1ab are co-translationally or post-transiationally processed into at least 16 distinct non-structural proteins designated nsp1-16 and include papain-like cysteine protease (PLpro or nsp3), main 3C-like cysteine protease (3CLpro or nsp5); RNA-dependent RNA polymerase (RdRp or nsp12), helicase (Hel or nsp 13), 3′→5′ exonuclease (ExoN or nsp 14), nidovirus undylate-specific endoribonuclease (NendoU or nsp 15) and ribose-2′-O-methyltransferase (2′OMT or nsp 16).

Downstream of pp1ab, and located at the 3′-end of the genome are seven other genes encoding structural proteins, namely the spike (S) protein, membrane (M) protein and the envelope (E) protein (collectively the envelope proteins), the nucleocapsid (N) protein, and the accessory ORF3 protein. A select subset of coronaviruses (e.g., Betacoronavirus subgroup A members) possess an additional envelope protein called the hemagglutinin-esterase (HE) protein. A 180 kDa to 220 kDa glycoprotein, the S protein makes up large surface projections called peplomers. The M protein is a 27 kDa to 32 kDa glycoprotein that forms a triple-spanning transmembrane protein. The smallest structural protein, the E protein is about 7 kDa and appears to be involved in viral budding. The N protein is a phosphoprotein which is responsible for the helical symmetry of the nucleocapsid that encloses the genomic RNA.

The viral envelope is a lipid bilayer, obtained by budding through membranes of the endoplasmic reticulum (ER) or Golgi apparatus, in which the M, E, and S structural proteins are anchored. The E and M protein combined with the lipid bilayer to shape the viral envelope and maintain its size while the S protein is for interaction with the host cells.

Infection begins when the S protein attaches to its complementary host cell receptor, such as a metalloprotease amino peptidase N. Viruses with the HE protein can also bind on N-acyl neuraminic acid that serves as a co-receptor. After attachment, a protease of the host cell cleaves and activates the receptor-attached S protein. Depending on the host cell protease available, the virus to enter the host cell by receptor-mediated endocytosis or direct fusion of the viral envelope with the host membrane. On host cell entry, the virus particle is uncoated, and its genome is released into the cell cytoplasm. Since the genome is a positive-sense ssRNA it is directly translated by the host cell's ribosomes. Initially, RnRp is preferentially translated by the host's ribosomes which then enables RnRp to use the positive-sense ssRNA genome as a template to synthesize minus-sense ssRNA. The RnRp the utilises the minus-sense ssRNA for both replication: of new positive-sense ssRNA genomes for viral progeny and as a template to transcribe smaller subgenomic (sg) positive ssRNAs which are used to synthesize all other viral proteins.

Upon their expression, PLpro (nsp3) and 3CLpro (nsp5) begin cleaving the polyprotein at different specific site to yield the 16 nonstructural proteins. A number of the non-structural proteins, including RdRp, coalesce to form a multi-protein replicase-transcriptase complex (RTC) which functions in replication and transcription of negative-sense ssRNA from an RNA strand. The mRNAs encoding the seven structural proteins are translated by the host's ribosomes inside the endoplasmic reticulum. Once processed, the S, E, and M proteins, as well as the HE protein if present, transit through the secretory pathway into the Golgi intermediate compartment where these envelop proteins integrate into the lipid bilayer membrane of the ER. The N protein binds the newly synthesized genomic RNA in the cytoplasm and the assembled nucleocapsid then binds to the M protein located on the ER membrane. Binding initiates a budding process into the lumen of the ER where the assembled nucleocapsid is encased with its membrane to produce viral progeny. Progeny viruses are transported by Golgi vesicles to the cell membrane and released from the host cell by exocytosis through secretory vesicles into the extracellular space. Once released the viruses can infect other host cells.

The present disclosure provides a COV antigen or combination of COV antigens. An antigen is a molecule that elicits an immune response and includes, without limitation, proteins, polypeptides, peptides, polysaccharides and conjugates of lipids, such as, e.g., lipoproteins and glycolipids. A COV peptide antigen is any antigen that can elicit an immune response against a species, strain, genotype, subgenotype, serotype or genetic/antigenic lineage of a Coronavirus or component of a species, a strain, genotype, subgenotype, serotype or genetic/antigenic lineage of a Coronavirus. A Coronavirus includes, without limitation, a genus, a subgenera or a species from the subfamily Orthocoronavirinae of the family Coronavirinae. There are four genera of Orthocoronavirinae, Alphacoronaviruses (Alpha-CoV), Betacoronaviruses (Beta-CoV), Gammacoronaviruses (Gamma-CoV), and Deltacoronaviruses (Delta-CoV). Subgenera of Alpha-CoV include, without limitation, Colacovirus, Decacovirus, Duvinacovirus, Luchacovirus, Minacovirus, Minunacovirus, Myotacovirus, Nyctacovirus, Pedacovirus, Rhinacovirus, Setracovirus, Soracovirus, Sunacovirus, and Tegacovirus. Subgenera of Beta-CoV include, without limitation, Embecovirus, Hibecovirus, Merbecovirus, Nobecovirus, and Sarbecovirus. Subgenera of Gamma-CoV include, without limitation, Brangacovirus, Cegacovirus, and Igacovirus. Subgenera of Delta-CoV include, without limitation, Andecovirus, Buldecovirus, and Herdecovirus. A component of a species, a strain, a genotype, a subgenotype, a serotype or a genetic/antigenic lineage of a Coronavirus includes, without limitation, the genome of a Coronavirus, a protein encoded by the genome of a Coronavirus, and a lipid membrane component of a Coronavirus. In an aspect of this embodiment, COV peptide antigen is any antigen that can elicit an immune response against a single Coronavirus species, strain, genotype, subgenotype, serotype or genetic/antigenic lineage or component of a Coronavirus species, strain, genotype, subgenotype, serotype or genetic/antigenic lineage. In another aspect of this embodiment, Coronavirus peptide antigen is any antigen that can elicit an immune response against two or more Coronavirus strains, genotypes, subgenotypes, serotypes or genetic/antigenic lineages or components from two or more Coronavirus strains, genotypes, subgenotypes, serotypes or genetic/antigenic lineages.

A COV peptide antigen must be large enough to be substantially unique in sequence, thus reducing the possibility of evoking T-cell response or B-cell response which produces antibodies that are cross-reactive against antigens other than a COV peptide antigen disclosed herein. Typically, a COV peptide antigen disclosed herein has a length of about 5 to about 60 amino acids. In aspects of this embodiment, a COV peptide antigen disclosed herein may have a length of, e.g., about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 26, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60 amino acids. In other aspects of this embodiment, a COV peptide antigen disclosed herein may have a length of, e.g., at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, at least 55, at least 60 amino acids. In yet other aspects of this embodiment, a COV peptide antigen disclosed herein may have a length of, e.g., at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, at most 60 amino acids.

In still other aspects of this embodiment, a COV peptide antigen disclosed herein may have a length of, e.g., between about 7 to about 10 amino acids, about 7 to about 12 amino acids, about 7 to about 15 amino acids, about 7 to about 18 amino acids, about 7 to about 20 amino acids, about 7 to about 25 amino acids, about 7 to about 30 amino acids, about 7 to about 35 amino acids, about 7 to about 40 amino acids, about 7 to about 45 amino acids, about 7 to about 50 amino acids, about 7 to about 55 amino acids, about 7 to about 60 amino acids, about 10 to about 12 amino acids, about 10 to about 15 amino acids, about 10 to about 18 amino acids, about 10 to about 20 amino acids, about 10 to about 25 amino acids, about 10 to about 30 amino acids, about 10 to about 35 amino acids, about 10 to about 40 amino acids, about 10 to about 45 amino acids, about 10 to about 50 amino acids, about 10 to about 55 amino acids, about 10 to about 60 amino acids, about 12 to about 15 amino acids, about 12 to about 18 amino acids, about 12 to about 20 amino acids, about 12 to about 25 amino acids, about 12 to about 30 amino acids, about 12 to about 35 amino acids, about 12 to about 40 amino acids, about 12 to about 45 amino acids, about 12 to about 50 amino acids, about 12 to about 55 amino acids, about 12 to about 60 amino acids, about 15 to about 18 amino acids, about 15 to about 20 amino acids, about 15 to about 25 amino acids, about 15 to about 30 amino acids, about 15 to about 35 amino acids, about 15 to about 40 amino acids, about 15 to about 45 amino acids, about 15 to about 50 amino acids, about 15 to about 55 amino acids, about 15 to about 60 amino acids, about 18 to about 20 amino acids, about 18 to about 25 amino acids, about 18 to about 30 amino acids, about 18 to about 35 amino acids, about 18 to about 40 amino acids, about 18 to about 45 amino acids, about 18 to about 50 amino acids, about 18 to about 55 amino acids, about 18 to about 60 amino acids, about 20 to about 25 amino acids, about 20 to about 30 amino acids, about 20 to about 35 amino acids, about 20 to about 40 amino acids, about 20 to about 45 amino acids, about 20 to about 50 amino acids, about 20 to about 55 amino acids, about 20 to about 60 amino acids, about 25 to about 30 amino acids, about 25 to about 35 amino acids, about 25 to about 40 amino acids, about 25 to about 45 amino acids, about 25 to about 50 amino acids, about 25 to about 55 amino acids, or about 25 to about 60 amino acids.

In some embodiments, a COV peptide antigen comprises an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12 (see Table 1). In aspects of this embodiment, a COV peptide antigen disclosed herein has an amino acid identity of, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In yet other aspects of this embodiment, a COV peptide antigen disclosed herein has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.

TABLE 1
COV Peptide Antigens

| Protein | SEQ ID NO: | Amino Acid Sequence | Length |
|---|---|---|---|
| RdRp-α | 1 | TKRNVLPTMTQLNLKYAISGKERARTV | 27 |
| RdRp-βγ | 2 | TKRNVIPTITQMNLKYAISAKNRARTV | 27 |
| RdRp-βδ | 3 | TKRNVLPTLTQMNLKYAISAKNRARTV | 27 |
| Mα | 4 | PTGVTLTLLSGNLYAEGFK | 19 |
| Mα | 5 | PTGVTLTLLSGTLLVEGYK | 19 |
| Mα | 6 | PTGITVTLLSGVLYVDGHR | 19 |
| Mα | 7 | PTGVTLTLLSGVLLVDGHK | 19 |
| Mα | 8 | PTGITLTILSGTLFFDGIRIA | 19 |
| Mβ | 9 | YFIASFRLFARTRSMWSFNPETN | 23 |
| Mβ | 10 | YFVNSIRLFIRTGSWWSFNPETN | 23 |
| Mγ | 11 | GYWIQSIRLFKRCRSWWSFNPESN | 24 |
| Mδ | 12 | KLILLWLLQPFTLVVTIW | 18 |

In other aspects of this embodiment, a COV peptide antigen disclosed herein comprises an amino acid sequence having a length of, e.g., at least 5 amino acids, at least 6 amino acids, at least 7 amino acids, at least 8 amino acids, at least 9 amino acids, at least 10 amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, at least 21 amino acids, at least 22 amino acids, at least 23 amino acids, at least 24 amino acids, at least 25 amino acids, at least 26 amino acids, at least 27 amino acids, at least 28 amino acids, at least 29 amino acids, at least 30 amino acids, at least 31 amino acids, at least 32 amino acids, at least 33 amino acids, at least 34 amino acids, at least 35 amino acids, at least 36 amino acids, at least 37 amino acids, at least 38 amino acids, at least 39 amino acids, at least 40 amino acids, at least 41 amino acids, at least 42 amino acids, at least 43 amino acids, at least 44 amino acids, at least 45 amino acids, at least 46 amino acids, at least 47 amino acids, at least 48 amino acids, at least 49 amino acids, at least 50 amino acids, at least 51 amino acids, at least 52 amino acids, at least 53 amino acids, at least 54 amino acids, at least 55 amino acids, at least 56 amino acids, at least 57 amino acids, at least 58 amino acids, at least 59 amino acids, at least 60 amino acids, the amino acid sequence being taken from a contiguous amino acid subsequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In yet other aspects of this embodiment, a COV peptide antigen disclosed herein comprises an amino acid sequence having a length of, e.g., at most 5 amino acids, at most 6 amino acids, at most 7 amino acids, at most 8 amino acids, at most 9 amino acids, at most 10 amino acids, at most 11 amino acids, at most 12 amino acids, at most 13 amino acids, at most 14 amino acids, at most 15 amino acids, at most 16 amino acids, at most 17 amino acids, at most 18 amino acids, at most 19 amino acids, at most 20 amino acids, at most 21 amino acids, at most 22 amino acids, at most 23 amino acids, at most 24 amino acids, at most 25 amino acids, at most 26 amino acids, at most 27 amino acids, at most 28 amino acids, at most 29 amino acids, at most 30 amino acids, at most 31 amino acids, at most 32 amino acids, at most 33 amino acids, at most 34 amino acids, at most 35 amino acids, at most 36 amino acids, at most 37 amino acids, at most 38 amino acids, at most 39 amino acids, at most 40 amino acids, at most 41 amino acids, at most 42 amino acids, at most 43 amino acids, at most 44 amino acids, at most 45 amino acids, at most 46 amino acids, at most 47 amino acids, at most 48 amino acids, at most 49 amino acids, at most 50 amino acids, at most 51 amino acids, at most 52 amino acids, at most 53 amino acids, at most 54 amino acids, at most 55 amino acids, at most 56 amino acids, at most 57 amino acids, at most 58 amino acids, at most 59 amino acids, at most 60 amino acids, the amino acid sequence being taken from a contiguous amino acid subsequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.

In other aspects of this embodiment, a COV peptide antigen disclosed herein has, e.g., at least 1, 2, 3, 4, 5, 6, or 7 contiguous amino acid deletions, additions, and/or substitutions relative to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12; or at most 1, 2, 3, 4, 5, 6, or 7 contiguous amino acid deletions, additions, and/or substitutions relative to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In yet other aspects of this embodiment, a COV peptide antigen disclosed herein has, e.g., at least 1, 2, 3, 4, 5, 6, or 7 non-contiguous amino acid deletions, additions, and/or substitutions relative to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12; or at most 1, 2, 3, 4, 5, 6, or 7 non-contiguous amino acid deletions, additions, and/or substitutions relative to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art and from the teaching herein.

Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein Sequence Alignments by Iterative Refinement as Assessed by Reference to Structural Alignments, 264(4) J. Mol. Biol. 823-838 (1996).

Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, Bioinformatics 20(9):1428-1435 (2004).

Hybrid methods combine functional aspects of both global and local alignment methods. Non-limiting methods include, e.g., segment-to-segment comparison, see, e.g., Burkhard Morgenstern et al., Multiple DNA and Protein Sequence Alignment Based on Segment-To-Segment Comparison, 93(22) Proc. Natl. Acad. Sci. U.S.A. 1 2098-12103 (1996); T-Coffee, see, e.g., Cédric Notredame et al., T-Coffee: A Novel Algorithm for Multiple Sequence Alignment, 302(1) J. Mol. Biol. 205-217 (2000); MUSCLE, see, e.g., Robert C. Edgar, MUSCLE: Multiple Sequence Alignment With High Score Accuracy and High Throughput, 32(5) Nucleic Acids Res. 1792-1797 (2004); and DIALIGN-T, see, e.g., Amarendran R Subramanian et al., DIALIGN-T: An Improved Algorithm for Segment-Based Multiple Sequence Alignment, 6(1) BMC Bioinformatics 66 (2005).

The present specification describes various polypeptide variants where one amino acid is substituted for another, such as, e.g., a COV peptide antigen disclosed herein. A substitution can be assessed by a variety of factors, such as, e.g., the physic properties of the amino acid being substituted (Table 2) or how the original amino acid would tolerate a substitution (Table 3). The selections of which amino acid can be substituted for another amino acid in a polypeptide are known to a person of ordinary skill in the art.

TABLE 2
Amino Acid Properties

| Property | Amino Acids |
|---|---|
| Aliphatic | G, A, I, L, M, P, V |
| Aromatic | F, H, W, Y |
| C-beta branched | I, V, T |
| Hydrophobic | C, F, I, L, M, V, W |
| Small polar | D, N, P |
| Small non-polar | A, C, G, S, T |
| Large polar | E, H, K, Q, R, W, Y |
| Large non-polar | F, I, L, M, V |
| Charged | D, E, H, K, R |
| Uncharged | C, S, T |
| Negative | D, E |
| Positive | H, K, R |
| Acidic | D, E |
| Basic | K, R |
| Amide | N, Q |

TABLE 3
Amino Acid Substitutions

| Amino | Favored | | |
|---|---|---|---|
| Acid | Substitution | Neutral Substitutions | Disfavored substitution |
| A | G, S, T | C, E, I, K, M, L, P, Q, R, V | D, F, H, N, Y, W |
| C | F, S, Y, W | A, H, I, M, L, T, V | D, E, G, K, N, P, Q, R |
| D | E, N | G, H, K, P, Q, R, S, T | A, C, I, L, |
| E | D, K, Q | A, H, N, P, R, S, T | C, F, G, I, L, M, V, W, Y |
| F | M, L, W, Y | C, I, V | A, D, E, G, H, K, N, P, Q, R, S, T |
| G | A, S | D, K, N, P, Q, R | C, E, F, H, I, L, M, T, V, W, Y |
| H | N, Y | C, D, E, K, Q, R, S, T, W | A, F, G, I, L, M, P, V |
| I | V, L, M | A, C, T, F, Y | D, E, G, H, K, N, P, Q, R, S, W |
| K | Q, E, R | A, D, G, H, M, N, P, S, T | C, F, I, L, V, W, Y |
| L | F, I, M, V | A, C, W, Y | D, E, G, H, K, N, P, Q, R, S, T |
| M | F, I, L, V | A, C, R, Q, K, T, W, Y | D, E, G, H, N, P, S |
| N | D, H, S | E, G, K, Q, R, T | A, C, F, I, L, M, P, V, W, Y |
| P | — | A, D, E, G, K, Q, R, S, T | C, F, H, I, L, M, N, V, W, Y |
| Q | E, K, R | A, D, G, H, M, N, P, S, T | C, F, I, L, V, W, Y |
| R | K, Q | A, D, E, G, H, M, N, P, S, T | C, F, I, L, V, W, Y |
| S | A, N, T | C, D, E, G, H, K, P, Q, R, T | F, I, L, M, V, W, Y |
| T | S | A, C, D, E, H, I, K, M, N, P, Q, | F, G, L, W, Y |
| | | R, V | |
| V | I, L, M | A, C, F, T, Y | D, E, G, H, K, N, P, Q, R, S, W |
| W | F, Y | H, L, M | A, C, D, E, G, I, K, N, P, Q, R, S, |
| | | | T, V |
| Y | F, H, W | C, I, L, M, V | A, D, E, G, K, N, P, Q, R, S, T |

Matthew J. Betts and Robert, B. Russell, Amino Acid Properties and Consequences of Substitutions, pp. 289-316, In Bioinformatics for Geneticists, (eds Michael R. Barnes, Ian C. Gray, Wiley, 2003).

In aspects of this embodiment, a hydrophobic amino acid at one particular position in a COV peptide antigen disclosed herein can be substituted with another hydrophobic amino acid. Examples of hydrophobic amino acids include, e.g., C, F, I, L, M, V and W. In another aspect of this embodiment, an aliphatic amino acid at one particular position in a COV peptide antigen disclosed herein can be substituted with another aliphatic amino acid. Examples of aliphatic amino acids include, e.g., A, I, L, P, and V. In yet another aspect of this embodiment, an aromatic amino acid at one specific position in a COV peptide antigen disclosed herein can be substituted with another aromatic amino acid. Examples of aromatic amino acids include, e.g., F, H, W and Y. In still another aspect of this embodiment, a stacking amino acid at one specific position in a COV peptide antigen disclosed herein can be substituted with another stacking amino acid. Examples of stacking amino acids include, e.g., F, H, W and Y. In a further aspect of this embodiment, a polar amino acid at one particular position in a COV peptide antigen disclosed herein can be substituted with another polar amino acid. Examples of polar amino acids include, e.g., D, E, K, N, Q, and R. In a further aspect of this embodiment, a less polar or indifferent amino acid at one particular position in a COV peptide antigen disclosed herein can be substituted with another less polar or indifferent amino acid. Examples of less polar or indifferent amino acids include, e.g., A, H, G, P, S, T, and Y. In a yet further aspect of this embodiment, a positive charged amino acid at one particular position in a COV peptide antigen disclosed herein can be substituted with another positive charged amino acid. Examples of positive charged amino acids include, e.g., K, R, and H. In a still further aspect of this embodiment, a negative charged amino acid at one particular position in a COV peptide antigen disclosed herein can be substituted with another negative charged amino acid. Examples of negative charged amino acids include, e.g., D and E. In another aspect of this embodiment, a small amino acid at one particular position in a COV peptide antigen disclosed herein can be substituted with another small amino acid. Examples of small amino acids include, e.g., A, D, G, N, P, S, and T. In yet another aspect of this embodiment, a C-beta branching amino acid at one particular position in a COV peptide antigen disclosed herein can be substituted with another C-beta branching amino acid. Examples of C-beta branching amino acids include, e.g., I, T and V.

Aspects of the present disclosure comprise, in part, a broad-spectrum, pan-broad-spectrum, pan-COV immunogenic composition. A broad-spectrum, pan-COV immunogenic composition disclosed herein can be, without limitation, an amino acid-based immunogenic composition or a nucleotide-based immunogenic composition. A broad-spectrum, pan-COV immunogenic composition disclosed herein may optionally also include one or more adjuvants, encoded/included in a construct provided in accordance with the present disclosure and/or included as a component in a broad-spectrum, pan-COV immunogenic composition as disclosed herein.

A broad-spectrum, pan-COV immunogenic composition disclosed herein may be used to prevent infection by a Coronavirus species, strain, genotype, subgenotype, serotype or genetic/antigenic lineage. In some embodiments, a broad-spectrum, pan-COV immunogenic composition disclosed herein when administered to an individual, elicits, evokes, or otherwise stimulates an immune response against one or more Coronavirus species, strains, genotypes, subgenotypes, serotypes or genetic/antigenic lineages. In some embodiments, a broad-spectrum, pan-COV immunogenic composition disclosed herein, when administered to an individual, elicits, evokes or otherwise stimulates an immune response against one Coronavirus species, strain, genotype, subgenotype, serotype or genetic/antigenic lineage to produce cellular and/or humoral response capable of recognizing infected cells inducing cytotoxicity. Some antibodies may also have viral neutralizing activity. In some embodiment, a broad-spectrum, pan-COV immunogenic composition disclosed herein, when administered to an individual, elicits, evokes or otherwise stimulates an immune response against two or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

An immune response disclosed herein is any response by the immune system of an individual to a broad-spectrum, pan-COV immunogenic composition disclosed herein. Exemplary immune responses include, but are not limited to, cellular and humoral immunity, such as, e.g., CTL responses, including antigen-specific induction of CD8+ CTLs, helper T-cell responses, including T-cell proliferative responses and cytokine production, and B-cell responses including, e.g., an antibody producing response. In an aspect of this embodiment, a broad-spectrum, pan-COV immunogenic composition is a COV vaccine.

In some embodiments, a broad-spectrum, pan-COV immunogenic composition disclosed herein comprises one or more COV peptide antigens disclosed herein. A COV peptide antigen can be a synthetic protein, polypetide, or peptide, a protein, polypetide, or peptide transcribed and/or translated from a nucleotide, as well as a peptidomimetic or any other a small protein-like chain designed to mimic a peptide. A peptidomimetic can be classified into four classes, Class A peptidomimetics which comprises modified peptides, Class B peptidomimetics which comprise modified peptides and peptidic foldamers, Class C peptidomimetics which comprise structural mimetics including foldamers, and Class D peptidomimetics which comprise mechanistic mimetids. Non-limiting examples of a peptidomimetic include peptoids, β-peptides, and D-peptides.

In some embodiments, a broad-spectrum, pan-COV immunogenic composition disclosed herein comprises one or more COV peptide antigens disclosed herein, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more COV peptide antigens disclosed herein, and when administered to an individual, the one or more COV peptide antigens elicit, evoke or otherwise stimulate an immune response against the one or more Coronavirus species, strains, genotypes, subgenotypes, serotypes or genetic/antigenic lineages. In aspects of these embodiments, a broad-spectrum, pan-COV immunogenic composition disclosed herein comprises one or more COV peptide antigens disclosed herein, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more COV peptide antigens disclosed herein, and when administered to an individual, the one or more COV peptide antigens elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises one or more COV peptide antigens disclosed herein, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more COV peptide antigens disclosed herein, where each COV peptide antigen is necessary to elicit, evoke or otherwise stimulate an immune response against one or more Coronavirus species, strains, genotypes, subgenotypes, serotypes or genetic/antigenic lineages. In aspects of these embodiments, a broad-spectrum, pan-COV immunogenic composition disclosed herein comprises one or more COV peptide antigens disclosed herein, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more COV peptide antigens disclosed herein, where each COV peptide antigen is necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a COV peptide antigen from a RNA dependent RNA polymerase from an alphacoronavirus species, strain, genotype, subgenotype, serotypes, or genetic/antigenic lineage disclosed herein, a RNA dependent RNA polymerase from a betacoronavirus species, strain, genotype, subgenotype, serotypes, or genetic/antigenic lineage disclosed herein, a RNA dependent RNA polymerase from a gammacoronavirus species, strain, genotype, subgenotype, serotypes, or genetic/antigenic lineage disclosed herein, a RNA dependent RNA polymerase from a deltacoronavirus species, strain, genotype, subgenotype, serotypes, or genetic/antigenic lineage disclosed herein, a M protein from an alphacoronavirus species, strain, genotype, subgenotype, serotypes, or genetic/antigenic lineage disclosed herein, a M protein from a betacoronavirus species, strain, genotype, subgenotype, serotypes, or genetic/antigenic lineage disclosed herein, a M protein from a gammacoronavirus species, strain, genotype, subgenotype, serotypes, or genetic/antigenic lineage disclosed herein, and/or a M protein from a deltacoronavirus species, strain, genotype, subgenotype, serotypes, or genetic/antigenic lineage disclosed herein, in any combination or order thereof. In some embodiments, a broad-spectrum, pan-COV immunogenic composition disclosed herein comprises 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more different COV peptide antigens, in any combination or order thereof, where each of the 2 or more different COV peptide antigens are from a RNA dependent RNA polymerase from an alphacoronavirus species, strain, genotype, subgenotype, serotypes, or genetic/antigenic lineage disclosed herein, a RNA dependent RNA polymerase from a betacoronavirus species, strain, genotype, subgenotype, serotypes, or genetic/antigenic lineage disclosed herein, a RNA dependent RNA polymerase from a gammacoronavirus species, strain, genotype, subgenotype, serotypes, or genetic/antigenic lineage disclosed herein, a RNA dependent RNA polymerase from a deltacoronavirus species, strain, genotype, subgenotype, serotypes, or genetic/antigenic lineage disclosed herein, a M protein from an alphacoronavirus species, strain, genotype, subgenotype, serotypes, or genetic/antigenic lineage disclosed herein, a M protein from a betacoronavirus species, strain, genotype, subgenotype, serotypes, or genetic/antigenic lineage disclosed herein, a M protein from a gammacoronavirus species, strain, genotype, subgenotype, serotypes, or genetic/antigenic lineage disclosed herein, and/or a M protein from a deltacoronavirus strain disclosed herein. In some embodiments, a broad-spectrum, pan-COV immunogenic composition disclosed herein comprises 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more different COV peptide antigens, in any combination or order thereof, where each of the 6 or more different COV peptide antigens are from a RNA dependent RNA polymerase from an alphacoronavirus species, strain, genotype, subgenotype, serotypes, or genetic/antigenic lineage disclosed herein, a RNA dependent RNA polymerase from a betacoronavirus species, strain, genotype, subgenotype, serotypes, or genetic/antigenic lineage disclosed herein, a RNA dependent RNA polymerase from a gammacoronavirus species, strain, genotype, subgenotype, serotypes, or genetic/antigenic lineage disclosed herein, a RNA dependent RNA polymerase from a deltacoronavirus species, strain, genotype, subgenotype, serotypes, or genetic/antigenic lineage disclosed herein, a M protein from an alphacoronavirus species, strain, genotype, subgenotype, serotypes, or genetic/antigenic lineage disclosed herein, a M protein from a betacoronavirus species, strain, genotype, subgenotype, serotypes, or genetic/antigenic lineage disclosed herein, a M protein from a gammacoronavirus species, strain, genotype, subgenotype, serotypes, or genetic/antigenic lineage disclosed herein, and/or a M protein from a deltacoronavirus species, strain, genotype, subgenotype, serotypes, or genetic/antigenic lineage disclosed herein. In some embodiments, a broad-spectrum, pan-COV immunogenic composition disclosed herein comprises 12 or more different COV peptide antigens, such as 13 or more, 14 or more, 15 or more, 16 or more, 17 or more, 18 or more, 19 or more, 20 or more, 21 or more, 22 or more, 23 or more, 24 or more COV peptide antigens, in any combination or order thereof, where each of the 12 or more different COV peptide antigens are from a RNA dependent RNA polymerase from an alphacoronavirus species, strain, genotype, subgenotype, serotypes, or genetic/antigenic lineage disclosed herein, a RNA dependent RNA polymerase from a betacoronavirus species, strain, genotype, subgenotype, serotypes, or genetic/antigenic lineage disclosed herein, a RNA dependent RNA polymerase from a gammacoronavirus species, strain, genotype, subgenotype, serotypes, or genetic/antigenic lineage disclosed herein, a RNA dependent RNA polymerase from a deltacoronavirus species, strain, genotype, subgenotype, serotypes, or genetic/antigenic lineage disclosed herein, a M protein from an alphacoronavirus species, strain, genotype, subgenotype, serotypes, or genetic/antigenic lineage disclosed herein, a M protein from a betacoronavirus species, strain, genotype, subgenotype, serotypes, or genetic/antigenic lineage disclosed herein, a M protein from a gammacoronavirus species, strain, genotype, subgenotype, serotypes, or genetic/antigenic lineage disclosed herein, and/or a M protein from a deltacoronavirus species, strain, genotype, subgenotype, serotypes, or genetic/antigenic lineage disclosed herein. In some embodiments, a broad-spectrum, pan-COV immunogenic composition disclosed herein comprises 18 or more different COV peptide antigens, such as 19 or more, 20 or more, 21 or more, 22 or more, 23 or more, 24 or more COV peptide antigens, in any combination or order thereof, where each of the 18 or more different COV peptide antigens are from a RNA dependent RNA polymerase from an alphacoronavirus species, strain, genotype, subgenotype, serotypes, or genetic/antigenic lineage disclosed herein, a RNA dependent RNA polymerase from a betacoronavirus species, strain, genotype, subgenotype, serotypes, or genetic/antigenic lineage disclosed herein, a RNA dependent RNA polymerase from a gammacoronavirus species, strain, genotype, subgenotype, serotypes, or genetic/antigenic lineage disclosed herein, a RNA dependent RNA polymerase from a deltacoronavirus species, strain, genotype, subgenotype, serotypes, or genetic/antigenic lineage disclosed herein, a M protein from an alphacoronavirus species, strain, genotype, subgenotype, serotypes, or genetic/antigenic lineage disclosed herein, a M protein from a betacoronavirus species, strain, genotype, subgenotype, serotypes, or genetic/antigenic lineage disclosed herein, a M protein from a gammacoronavirus species, strain, genotype, subgenotype, serotypes, or genetic/antigenic lineage disclosed herein, and/or a M protein from a deltacoronavirus species, strain, genotype, subgenotype, serotypes, or genetic/antigenic lineage disclosed herein. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more different COV peptide antigens, in any combination or order thereof, where at least one of the COV peptide antigens is from a RNA dependent RNA polymerase from an alphacoronavirus species, strain, genotype, subgenotype, serotypes, or genetic/antigenic lineage disclosed herein, at least one of the COV peptide antigens is from a RNA dependent RNA polymerase from a betacoronavirus species, strain, genotype, subgenotype, serotypes, or genetic/antigenic lineage disclosed herein, at least one of the COV peptide antigens is from a RNA dependent RNA polymerase from a gammacoronavirus species, strain, genotype, subgenotype, serotypes, or genetic/antigenic lineage disclosed herein, at least one of the COV peptide antigens is from a RNA dependent RNA polymerase from a deltacoronavirus species, strain, genotype, subgenotype, serotypes, or genetic/antigenic lineage disclosed herein, at least one of the COV peptide antigens is from a M protein from an alphacoronavirus species, strain, genotype, subgenotype, serotypes, or genetic/antigenic lineage disclosed herein, at least one of the COV peptide antigens is from a M protein from a betacoronavirus species, strain, genotype, subgenotype, serotypes, or genetic/antigenic lineage disclosed herein, at least one of the COV peptide antigens is from a M protein from a gammacoronavirus species, strain, genotype, subgenotype, serotypes, or genetic/antigenic lineage disclosed herein, and/or at least one of the COV peptide antigens is from a M protein from a deltacoronavirus species, strain, genotype, subgenotype, serotypes, or genetic/antigenic lineage disclosed herein. In some embodiments, a broad-spectrum, pan-COV immunogenic composition disclosed herein comprises 12 or more different COV peptide antigens, such as 13 or more, 14 or more, 15 or more, 16 or more, 17 or more, 18 or more, 19 or more, 20 or more, 21 or more, 22 or more, 23 or more, 24 or more different COV peptide antigens, in any combination or order thereof, where at least one of the COV peptide antigens is from a RNA dependent RNA polymerase from an alphacoronavirus species, strain, genotype, subgenotype, serotypes, or genetic/antigenic lineage disclosed herein, at least one of the COV peptide antigens is from a RNA dependent RNA polymerase from a betacoronavirus species, strain, genotype, subgenotype, serotypes, or genetic/antigenic lineage disclosed herein, at least one of the COV peptide antigens is from a RNA dependent RNA polymerase from a gammacoronavirus species, strain, genotype, subgenotype, serotypes, or genetic/antigenic lineage disclosed herein, at least one of the COV peptide antigens is from a RNA dependent RNA polymerase from a deltacoronavirus species, strain, genotype, subgenotype, serotypes, or genetic/antigenic lineage disclosed herein, at least one of the COV peptide antigens is from a M protein from an alphacoronavirus species, strain, genotype, subgenotype, serotypes, or genetic/antigenic lineage disclosed herein, at least one of the COV peptide antigens is from a M protein from a betacoronavirus species, strain, genotype, subgenotype, serotypes, or genetic/antigenic lineage disclosed herein, at least one of the COV peptide antigens is from a M protein from a gammacoronavirus species, strain, genotype, subgenotype, serotypes, or genetic/antigenic lineage disclosed herein, and/or at least one of the COV peptide antigens is from a M protein from a deltacoronavirus species, strain, genotype, subgenotype, serotypes, or genetic/antigenic lineage disclosed herein. In some embodiments, a broad-spectrum, pan-COV immunogenic composition disclosed herein comprises 18 or more different COV peptide antigens, such as 19 or more, 20 or more, 21 or more, 22 or more, 23 or more, 24 or more different COV peptide antigens, in any combination or order thereof, where at least one of the COV peptide antigens is from a RNA dependent RNA polymerase from an alphacoronavirus species, strain, genotype, subgenotype, serotypes, or genetic/antigenic lineage disclosed herein, at least one of the COV peptide antigens is from a RNA dependent RNA polymerase from a betacoronavirus species, strain, genotype, subgenotype, serotypes, or genetic/antigenic lineage disclosed herein, at least one of the COV peptide antigens is from a RNA dependent RNA polymerase from a gammacoronavirus species, strain, genotype, subgenotype, serotypes, or genetic/antigenic lineage disclosed herein, at least one of the COV peptide antigens is from a RNA dependent RNA polymerase from a deltacoronavirus species, strain, genotype, subgenotype, serotypes, or genetic/antigenic lineage disclosed herein, at least one of the COV peptide antigens is from a M protein from an alphacoronavirus species, strain, genotype, subgenotype, serotypes, or genetic/antigenic lineage disclosed herein, at least one of the COV peptide antigens is from a M protein from a betacoronavirus species, strain, genotype, subgenotype, serotypes, or genetic/antigenic lineage disclosed herein, at least one of the COV peptide antigens is from a M protein from a gammacoronavirus species, strain, genotype, subgenotype, serotypes, or genetic/antigenic lineage disclosed herein, and/or at least one of the COV peptide antigens is from a M protein from a deltacoronavirus species, strain, genotype, subgenotype, serotypes, or genetic/antigenic lineage disclosed herein. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 2 or more different COV peptide antigens, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more different COV peptide antigens, where each of the 2 or more different COV peptide antigen has an amino acid sequence selected from the sequences of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more different COV peptide antigens, where each of the 6 or more different COV peptide antigens have the amino acid sequences of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 12 or more different COV peptide antigens, such as 13 or more, 14 or more, 15 or more, 16 or more, 17 or more, 18 or more, 19 or more, 20 or more, 21 or more, 22 or more, 23 or more, 24 or more different COV peptide antigens, where each of the 12 or more different COV peptide antigens have the amino acid sequences of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 18 different COV peptide antigens, such as 19 or more, 20 or more, 21 or more, 22 or more, 23 or more, 24 or more different COV peptide antigens, where each of the 18 different COV peptide antigens have the amino acid sequences of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a COV peptide antigen with the amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more different COV peptide antigens, where each of the 2 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more different COV peptide antigens, where each of the 6 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 12 or more different COV peptide antigens, such as 13 or more, 14 or more, 15 or more, 16 or more, 17 or more, 18 or more, 19 or more, 20 or more, 21 or more, 22 or more, 23 or more, 24 or more different COV peptide antigens, where each of the 12 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 18 different COV peptide antigens, such as 19 or more, 20 or more, 21 or more, 22 or more, 23 or more, 24 or more different COV peptide antigens, where each of the 18 different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprising one or more nucleotide sequences encoding a polypeptide including one or more COV peptide antigens disclosed herein. Upon administration, the one or more nucleotide sequences contained in a broad-spectrum, pan-COV immunogenic composition disclosed herein are transcribed and/or translated and provide the encoded polypeptide, including one or more COV peptide antigens (in whole or in part; separately or contiguously or in any combination of length, order or fragment) disclosed herein, in a cell. Such translation can occur in vivo, although such translation may occur ex vivo, in culture or in vitro if so desired, and the resultant peptide/protein being subsequently administered to a patient. A nucleotide sequence includes, without limitation, a DNA nucleotide sequence, a RNA nucleotide sequence including a mRNA nucleotide sequence, and a self-amplifying RNA nucleotide sequence.

A broad-spectrum, pan-COV immunogenic composition disclosed herein can be chemically synthesized or recombinantly produced using molecular biology procedures. A broad-spectrum, pan-COV immunogenic composition disclosed herein can comprise a vector used to cause, facilitate, or enhance delivery and/or expression of one or more nucleotide sequences encoding a polypeptide including one or more COV peptide antigens disclosed herein. In some embodiments, the vector is a viral vector used to cause, facilitate, or enhance delivery and/or expression of one or more nucleotide sequences encoding a polypeptide including one or more COV peptide antigens disclosed herein. Non-limiting viral vectors include DNA-based viral vectors, such as, e.g., an adenovirus-based vector, an adeno-associated virus-based vector, and herpes simplex virus-based vector, and RNA-based viral vectors, such as, e.g., an oncoretrovirus-based vector, a lentivirus-based vector, and human foamy virus-based vector. Examples of methods of making nucleotide sequences and expression constructs disclosed herein are described in, e.g., U.S. Pat. Nos. 9,192,661, 9,254,265, 9,872,900, 10,022,435, 10,023,626, 10,064,935, 10.124,055, 10,138,507, 10,155,029, 10,238,731, 10,272,150, 10,286,086, 10,407,683, 10,543,269, 10,590,161, 10,675,342, 10,695,419, 10,702,599, 10,702,600, 10,808,242, as well as U.S. Publication Nos. 2016/0017313, 2016/0024140, 2016/0024140, 2016/0032273, 2020/0069794, 2020/0222526, and 20200268873, the content of each of which is incorporated by reference in its entirety.

A nucleotide sequence disclosed herein can be codon optimized. As used herein, “codon optimized” refers to changing one or more codons of a nucleotide sequence without altering the amino acid sequence that it encodes in order to favour expression in a specific species. Codon optimization may be used to increase the abundance of the polypeptide that the nucleotide sequence encodes since “rare” codons are removed and replaced with abundant codons. In some embodiments, a nucleotide sequence disclosed herein may be codon optimized specifically for expression in humans. In some embodiments, a nucleotide sequence disclosed herein may be codon optimized specifically for expression in plants, bacteria, fungi, insect or mammalian cells used to translate the nucleotide sequence.

In some embodiments, a nucleotide sequence disclosed herein may optionally comprise or encode one or more than one modified histone stem-loop. The histone stem-loop is generally derived from histone genes and includes an intramolecular base pairing of two neighbored partially or entirely reverse complementary sequences separated by a spacer, including (e.g., consisting of) a short sequence, which forms the loop of the structure. The unpaired loop region is typically unable to base pair with either of the stem loop elements. It occurs more often in RNA, as is a key component of many RNA secondary structures but may be present in single-stranded DNA as well. Stability of the stem-loop structure generally depends on the length, number of mismatches or bulges, and base composition of the paired region; wobble base pairing (non-Watson-Crick base pairing) may result. In some embodiments, a nucleotide sequence from a broad-spectrum, pan-COV immunogenic composition disclosed herein may optionally have one or more AU-rich sequences or other destabilizing sequences found in the 3′UTR removed.

In some embodiments, a nucleotide sequence disclosed herein may optionally comprise one or more nucleoside or nucleotide modifications. Nucleotide sequence may comprise modifications that are naturally-occurring, non-naturally-occurring or the nucleotide sequence may comprise a combination of naturally-occurring and non-naturally-occurring modifications. Nucleotide sequences may include any useful modification, for example, of a sugar, a nucleobase, or an internucleoside linkage (e.g., to a linking phosphate, to a phosphodiester linkage or to the phosphodiester backbone). In some embodiments, a modified nucleotide sequence, introduced to a cell or organism, exhibits reduced degradation in the cell or organism, respectively, relative to an unmodified nucleotide sequence. In some embodiments, a modified nucleotide sequence, introduced into a cell or organism, may exhibit reduced immunogenicity in the cell or organism, respectively (e.g., a reduced innate response). Examples of nucleoside or nucleotide modifications and methods of incorporating such in a nucleotide sequence disclosed herein are described in, e.g., U.S. Pat. Nos. 9,872,900, 10,022,435, 10,023,626, 10,155,029, 10,286,086, 10,406,113, 10,407,683, 10,709,779, 10,808,242, and 10,815,291, the content of each of which is incorporated by reference in its entirety.

In some embodiments, a nucleotide sequence disclosed herein may optionally comprise a coding sequence for a signal peptide, the signal peptide coding sequence being operably linked to and is in frame with the coding sequence of a polypeptide disclosed herein. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. Signal peptides generally include three regions: an N-terminal region of differing length, which usually comprises positively charged amino acids; a hydrophobic region; and a short carboxy-terminal peptide region. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by a ER-resident signal peptidase of the host cell, or they remain uncleaved and function as a membrane anchor. A signal peptide may also facilitate the targeting of the protein to the cell membrane. Exemplary signal peptides, their sequences, lengths and origin are described in U.S. Pat. No. 10,064,934 which is hereby incorporated by reference in its entirety. In some embodiments, a signal peptide is present at the amino-terminus of a polypeptide disclosed herein. In some embodiments, a signal peptide is present at the carboxy-terminus of a polypeptide disclosed herein. The mature polypeptide produced by a broad-spectrum, pan-COV immunogenic composition disclosed herein typically does not comprise a signal peptide as it is cleaved from the nascent polypeptide at the cleavage junction during ER processing.

In some embodiments, a nucleotide sequence disclosed herein may optionally comprise a coding sequence for an adjuvant, the adjuvant coding sequence being operably linked to and is in frame with the coding sequence of a polypeptide disclosed herein. In some embodiments, an adjuvant thus encoded is a flagellin polypeptide. In aspects of these embodiments, a carboxy-terminus of a polypeptide disclosed herein is fused or linked to an amino terminus of a flagellin polypeptide. In other embodiments, an amino-terminus of a polypeptide disclosed herein is fused or linked to a carboxy-terminus of a flagellin polypeptide.

In some embodiments, a nucleotide sequence disclosed herein may optionally comprise a coding sequence for one or more glycosylation sites. N-linked glycans of viral proteins play important roles in modulating the immune response. Glycans can be important for maintaining the appropriate antigenic conformations, shielding potential neutralization epitopes, and may alter the proteolytic susceptibility of proteins. In some embodiments, a nucleotide sequence encoding a polypeptide produced by a broad-spectrum, pan-COV immunogenic composition disclosed herein can comprise one or more N-linked glycosylation sites. In some embodiments, a nucleotide sequence encoding a polypeptide produced by a broad-spectrum, pan-COV immunogenic composition disclosed herein can comprise a deletion or modification of one or more N-linked glycosylation sites.

In some embodiments, a nucleotide sequence disclosed herein can include at least one nucleic acid region encoding a binding peptide. Such a binding peptide is operably-linked in-frame to an open reading frame encoding a polypeptide disclosed herein as a fusion protein. In another aspect of this embodiment, a nucleotide sequence disclosed herein can include a plurality of nucleic acid regions encoding multiple operably-linked binding peptides. Therefore, aspects of this embodiment can include a nucleotide sequence disclosed herein including a one nucleic acid region encoding one or more operably-linked binding peptides, two or more operably-linked binding peptides, three or more operably-linked binding peptides, four or more operably-linked binding peptides, or five or more operably-linked binding peptides. In another aspect of this embodiment, nucleic acid regions comprising multiple binding peptides can encode multiple copies of the same binding peptide, different binding peptides, or any combination thereof. The location of a nucleic acid region encoding a binding peptide may be in various positions, including, without limitation, before the amino terminus of a polypeptide disclosed herein, within a polypeptide disclosed herein, or after the carboxy terminus of a polypeptide disclosed herein. Examples of binding peptides that can be encoded by a nucleic acid region disclosed in the present specification include, without limitation, epitope-binding peptides such as FLAG, Express™, human Influenza virus hemagluttinin (HA), human p62c-Myc protein (c-MYC), Vesicular Stomatitis Virus Glycoprotein (VSV-G), glycoprotein-D precursor of Herpes simplex virus (HSV), V5, AU1, and AUS; affinity-binding peptides such as polyhistidine (HIS), streptavidin binding peptide (strep), and biotin; and peptide-binding domains such as the glutathione binding domain of glutathione-S-transferase, the calmodulin binding domain of the calmodulin binding protein, and the maltose binding domain of the maltose binding protein. An exemplary sequence for a HIS tag is SEQ ID NO: 16. An exemplary sequence for a HA tag is SEQ ID NO: 17. Non-limiting examples of specific protocols for selecting, making and using an appropriate binding peptide are described in, e.g., Molecular Cloning A Laboratory Manual (Joseph Sambrook & David W. Russell eds., Cold Spring Harbor Laboratory Press, 3rd ed. 2001); Antibodies: A Laboratory Manual (Edward Harlow & David Lane, eds., Cold Spring Harbor Laboratory Press, 2nd ed. 1998); and Using Antibodies: A Laboratory Manual: Portable Protocol No. I (Edward Harlow & David Lane, Cold Spring Harbor Laboratory Press, 1998), which are hereby incorporated by reference. In addition, non-limiting examples of binding peptides as well as well-characterized reagents, conditions and protocols are readily available from commercial vendors that include, without limitation, BD Biosciences-Clontech, Palo Alto, Calif.; BD Biosciences Pharmingen, San Diego, Calif.; Invitrogen, Inc, Carlsbad, Calif.; QIAGEN, Inc., Valencia, Calif.; and Stratagene, La Jolla, Calif. These protocols are routine procedures within the scope of one skilled in the art and from the teaching herein.

In some embodiments, a broad-spectrum, pan-COV immunogenic composition disclosed herein comprises one nucleotide sequence encoding a polypeptide including one or more COV peptide antigens disclosed herein, in any combination or order thereof. In some embodiments, a nucleotide sequence contains a single open-reading frame which encodes a polypeptide, the polypeptide including one or more COV peptide antigens disclosed herein, in any combination or order thereof. In some embodiments, a nucleotide sequence can contain multiple open-reading frames, each multiple-reading frame encoding a different polypeptide, each of the different polypeptides including one or more COV peptide antigens disclosed herein, in any combination or order thereof. In some embodiments, a nucleotide sequence can contain multiple open-reading frames, with a first subset of the multiple-reading frame encoding the same polypeptide, the same polypeptide including one or more COV peptide antigens disclosed herein, in any combination or order thereof and a second subset of the multiple-reading frame encoding different polypeptide, each of the different polypeptides including one or more COV peptide antigens disclosed herein, in any combination or order thereof.

In some embodiments, a broad-spectrum, pan-COV immunogenic composition disclosed herein comprises two or more nucleotide sequences, where each nucleotide sequence encodes a polypeptide including one or more COV peptide antigens disclosed herein, in any combination or order thereof. In some embodiments, each of the two or more nucleotide sequences contain a single open-reading frame which encodes a polypeptide, the polypeptide including one or more COV peptide antigens disclosed herein, in any combination or order thereof. In some embodiments, each of the two or more nucleotide sequences contain multiple open-reading frames, each multiple-reading frame encoding a different polypeptide, each of the different polypeptides including one or more COV peptide antigens disclosed herein, in any combination or order thereof. In some embodiments, each of the two or more nucleotide sequences contain multiple open-reading frames, with a first subset of the multiple-reading frame encoding the same polypeptide, the same polypeptide including one or more COV peptide antigens disclosed herein, in any combination or order thereof and a second subset of the multiple-reading frame encoding different polypeptide, each of the different polypeptides including one or more COV peptide antigens disclosed herein, in any combination or order thereof. In some embodiments, a first subset of the two or more nucleotide sequences can each contain a single open-reading frame as disclosed herein and a second subset of the two or more nucleotide sequences can each contain multiple open-reading frames as disclosed herein.

In some embodiments, a nucleotide sequence disclosed herein may optionally comprise a coding sequence for an internal ribosome entry site (IRES). An IRES disclosed herein is an RNA element that allows for translation initiation in cap-independent manner. An IRES can function as a sole ribosome binding site, but it can also serve to provide a bi- or even multicistronic mRNA, each of which encodes COV peptide antigens disclosed herein, which are to be translated by the ribosomes independently of one another. Examples of IRES sequences, include, without limitation, IRES sequences from picornaviruses (e.g. FMDV), pestiviruses (CFFV), polioviruses (PV), encephalomyocarditis viruses (ECMV), foot and mouth disease viruses (FMDV), hepatitis C viruses (HCV), classical swine fever viruses (CSFV), mouse leukoma virus (MLV), simian immunodeficiency viruses (SIV) or cricket paralysis viruses (CrPV). In some embodiments, an IRES is operationally linked with each open-reading frame encoding a polypeptide disclosed herein and/or any other open reading frame contained within a nucleotide sequence disclosed herein, such as, e.g., an adjuvant disclosed herein. In some embodiments, an IRES is operationally linked at the 5′ end of each open-reading frame encoding a polypeptide disclosed herein and/or at the 5′ end of any other open reading frame contained within a nucleotide sequence disclosed herein, such as, e.g., an adjuvant disclosed herein.

In some embodiments, a nucleotide sequence disclosed herein may optionally encode or be a self-replicating nucleotide sequence. A self-replicating nucleotide sequence can enhance efficiency of RNA delivery and expression of the enclosed gene product including a polypeptide disclosed herein. For example, a self-replicating nucleotide sequence disclosed herein may encode a polypeptide disclosed herein which may raise the immune response. In some embodiments, a self-replicating nucleotide sequence disclosed herein can be designed so that the self-replicating nucleotide sequence does not induce production of infectious viral particles. As a non-limiting example, a self-replicating nucleotide sequence disclosed herein may be designed by the methods described in US Pub. No. 2011/0300205 and International Pub. No. WO 2011/005799, each of which is herein incorporated by reference in their entirety.

In some embodiments, a broad-spectrum, pan-COV immunogenic composition disclosed herein comprises a nucleotide sequence encoding a polypeptide including one or more COV peptide antigens disclosed herein, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more of COV peptide antigens disclosed herein, in any combination or order thereof.

In some embodiments, a broad-spectrum, pan-COV immunogenic composition comprises a nucleotide sequence encoding a polypeptide including one or more COV peptide antigens disclosed herein, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more or more COV peptide antigens disclosed herein, in any combination or order thereof, and when administered to an individual, the one or more COV peptide antigens elicit, evoke or otherwise stimulate an immune response against the one or more Coronavirus species, strains, genotypes, subgenotypes, serotypes or genetic/antigenic lineages. In aspects of these embodiments, a broad-spectrum, pan-COV immunogenic composition comprises a nucleotide sequence encoding a polypeptide including one or more COV peptide antigens disclosed herein, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more COV peptide antigens disclosed herein, in any combination or order thereof, and when administered to an individual, the one or more COV peptide antigens elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition comprises a nucleotide sequence encoding a polypeptide including one or more COV peptide antigens disclosed herein, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more COV peptide antigens disclosed herein, in any combination or order thereof, where each of the one or more COV peptide antigens is necessary to elicit, evoke or otherwise stimulate an immune response against one or more Coronavirus species, strains, genotypes, subgenotypes, serotypes or genetic/antigenic lineages. In aspects of these embodiments, a broad-spectrum, pan-COV immunogenic composition comprises a nucleotide sequence encoding a polypeptide including one or more COV peptide antigens disclosed herein, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more COV peptide antigens disclosed herein, in any combination or order thereof, where each of the one or more COV peptide antigens is necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a DNA nucleotide sequence encoding a RNA nucleotide sequence encoding a polypeptide including one or more COV peptide antigens disclosed herein. DNA broad-spectrum, pan-COV immunogenic compositions disclosed herein work by delivering genetically engineered expression constructs containing the DNA nucleotide sequence encoding one or more COV antigens disclosed herein into cells in a manner that enables cells to directly produce the encoded antigens, thus causing a protective immunological response. DNA broad-spectrum, pan-COV immunogenic compositions disclosed herein may induce T-cell response against such epitopes and may also induce a satisfactory concentration of neutralizing antibodies, i.e., a B cell response. Generally, procedures for how to make and use DNA vaccines can be found in, e.g., U.S. Pat. Nos. 8,003,113, 8,785,411, 8,916,174, 9,085,638, 9,393,396, 9,415,098, 9,415,099, 9,446,120, 9,505,806, 9,913,886, 10,426,830, 10,543,261, 10,888,612, each of which is hereby incorporated by reference in its entirety.

In some embodiments, a broad-spectrum, pan-COV immunogenic composition disclosed herein comprises a RNA nucleotide sequence encoding a polypeptide including one or more COV peptide antigens. RNA broad-spectrum, pan-COV immunogenic compositions disclosed herein work by delivering a mRNA nucleotide sequence encoding one or more COV antigens disclosed herein into immune cells in a manner that enables cells to directly produce the encoded antigens, thus causing a protective immunological response. RNA broad-spectrum, pan-COV immunogenic compositions disclosed herein may induce T-cell response against such epitopes and may also induce a satisfactory concentration of neutralizing antibodies, i.e., a B cell response. Generally, procedures for how to make and use RNA vaccines can be found in, e.g., U.S. Pat. Nos. 9,872,900, 10,022,435, 10,064,935, 10,124,055, 10,238,731, 10,383,937, 10,449,244, 10,517,940, 10,653,767, 10,675,342, 10,695,419, 10,702,599, 10,702,600, 10,925,958, each of which is hereby incorporated by reference in its entirety.

A RNA nucleotide sequence encompasses not only RNA molecules as expressed or found in nature, but also RNA molecules comprising ribonucleic acid analogs and derivatives as described herein or as known in the art. Such nucleic acid analogs and derivatives of RNA include ribonucleotide analogs or derivatives and ribonucleoside analogs or derivatives. A “ribonucleoside” includes a nucleoside base and a ribose sugar, and a “ribonucleotide” is a ribonucleoside with one, two or three phosphate moieties. A RNA nucleotide sequence can be modified in the nucleobase structure or in the ribose-phosphate backbone structure, e.g., as described herein below. Non-limiting examples of ribonucleotide analogs or derivatives include a bridged nucleic acid (BNA), a cyclohexenyl nucleic acid (CeNA), an ethylene nucleic acid (ENA), a 2′-fluoro-hexitol nucleic acid (FHNA), a glycol nucleic acid (GNA), a locked nucleic acid (LNA), including a LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), a peptide nucleic acid (PNA), and a threose nucleic acid (TNA). Non-limiting examples of ribonucleoside analogs or derivatives include a 2′-O-methyl modified nucleoside, a nucleoside comprising a 5′ phosphorothioate group, 5′ phosphate group, 5′ triphosate group, 5′ phosphorodithioate group, a terminal nucleoside linked to a cholesteryl derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, 2′-alkoxyalkyl-modified nucleoside e.g., (2′-O-methoxyethyl) nucleoside, a phosphorodiamidate nucleoside, a phosphoramidate (amidophosphate) nucleoside, a morpholino nucleoside, and a non-natural base comprising ribonucleoside. In some embodiments, a RNA nucleotide sequence disclosed herein include one or more PNAs that have the ability to form the required duplex structure and that permit or mediate the specific degradation of a target RNA via a RISC pathway or inhibit the function by steric effects such as translation arrest or modulation. In some embodiments, a RNA nucleotide sequence disclosed herein include one or more hydrolysis resistant ribonucleic acid analogs or derivatives. In aspects of these embodiments, a hydrolysis resistant ribonucleic acid analog or derivative include a 2′-O-methyl-substituted nucleoside, a morpholino nucleoside, a BNA, a LNA, a PNA, or any combination thereof.

A skilled artisan will appreciate that, except where otherwise noted, nucleotide sequence sequences set forth in the instant application will recite “T”s in a representative DNA sequence but where the sequence represents RNA (e.g., mRNA), the “T”s would be substituted for “U”s. Thus, any of the RNA nucleotide sequences encoded by a DNA identified by a particular sequence identification number may also comprise the corresponding RNA (e.g., mRNA) sequence encoded by the DNA, where each “T” of the DNA sequence is substituted with “U.” Nucleotide sequences of the present disclosure may function as mRNA but can be distinguished from wild-type mRNA in their functional and/or structural design features, which serve to overcome existing problems of effective polypeptide expression using nucleic-acid based therapeutics.

In some embodiments, a RNA nucleotide sequence can comprise at one or more ribonucleic acid analogs or derivatives. These ribonucleic acid analogs or derivatives, can be part of a RNA nucleotide sequence encoding any one or more of the COV peptide antigens, in any combination or order thereof, and in accordance with the present disclosure. In aspects of these embodiments, a RNA nucleotide sequence can comprises, e.g., at least 1, at least 2, at least 3, at least 4, at least 5, at least 6, at least 7, at least 8, at least 9, at least 10, at least 15, at least 20 or more ribonucleic acid analogs or derivatives. In aspects of these embodiments, a RNA nucleotide sequence can comprises, e.g., about 1 to about 3, about 1 to about 5, about 1 to about 7, about 1 to about 9, about 1 to about 11, about 1 to about 13, about 1 to about 15, about 1 to about 17, about 1 to about 19, about 1 to about 21, about 3 to about 5, about 3 to about 7, about 3 to about 9, about 3 to about 11, about 3 to about 13, about 3 to about 15, about 3 to about 17, about 3 to about 19, about 3 to about 21, about 5 to about 7, about 5 to about 9, about 5 to about 11, about 5 to about 13, about 5 to about 15, about 5 to about 17, about 5 to about 19, about 5 to about 21, about 7 to about 9, about 7 to about 11, about 7 to about 13, about 7 to about 15, about 7 to about 17, about 7 to about 19, about 7 to about 21, about 9 to about 11, about 9 to about 13, about 9 to about 15, about 9 to about 17, about 9 to about 19, about 9 to about 21, about 11 to about 13, about 11 to about 15, about 11 to about 17, about 11 to about 19, about 11 to about 21, ribonucleic acid analogs or derivatives.

A mRNA refers to any RNA nucleotide sequence having an open reading frame encoding at least one polypeptide (comprising naturally-occurring amino acids, non-naturally-occurring amino acids, and/or modified polymer of amino acids) and can be translated to produce the encoded polypeptide in vitro, in vivo, in situ or ex vivo. The basic components of an mRNA nucleotide sequence comprise at least one coding region, and typically includes a 5′ untranslated region (UTR), and a 3′ UTR. An “open reading frame” is a continuous stretch of mRNA nucleotide sequence that encodes a polypeptide that begins with a start codon (the first codon of an mRNA nucleotide sequence translated by a ribosome and typically is AUG for methionine), and ends with a stop codon (the codon of an mRNA nucleotide sequence that signals a termination of translation and typically is UAA, UAG or UGA). A “5′ untranslated region” (5′UTR) refers to a region of an mRNA that is directly upstream (i.e., 5′) from the start codon that does not encode a polypeptide and may or may not contain, a 5′ terminal cap, and/or an enhancer and/or promoter sequence, which may be modified or unmodified or which may be activated or inactivated. In some embodiments, a 5′ terminal cap structure includes, without limitation, 7mG(5′)ppp(5′)N,pN2p (cap 0), 7mG(5′)ppp(5)NImpNp (cap 1), and 7mG(5′)-ppp(5′)NImpN2mp (cap 2). A “3′ untranslated region” (3′UTR) refers to a region of an mRNA that is directly downstream (i.e., 3′) from the stop codon that does not encode a polypeptide and may or may not contain a polyadenylation sequence and/or histone stem-loop. In some embodiments, a 3′UTR includes both a polyadenylation sequence and histone stem-loop which act synergistically to increase the protein expression beyond the level observed with either of the individual elements.

In some embodiments, a broad-spectrum, pan-COV immunogenic composition disclosed herein comprises a nucleotide sequence (e.g., a DNA or RNA) encoding a polypeptide comprising one or more COV peptide antigens obtained from a RNA dependent RNA polymerase from an alphacoronavirus species, strain, genotype, subgenotype, serotypes, or genetic/antigenic lineage disclosed herein, a RNA dependent RNA polymerase from a betacoronavirus species, strain, genotype, subgenotype, serotypes, or genetic/antigenic lineage disclosed herein, a RNA dependent RNA polymerase from a gammacoronavirus species, strain, genotype, subgenotype, serotypes, or genetic/antigenic lineage disclosed herein, a RNA dependent RNA polymerase from a deltacoronavirus species, strain, genotype, subgenotype, serotypes, or genetic/antigenic lineage disclosed herein, a M protein from an alphacoronavirus species, strain, genotype, subgenotype, serotypes, or genetic/antigenic lineage disclosed herein, a M protein from a betacoronavirus species, strain, genotype, subgenotype, serotypes, or genetic/antigenic lineage disclosed herein, a M protein from a gammacoronavirus species, strain, genotype, subgenotype, serotypes, or genetic/antigenic lineage disclosed herein, and/or a M protein from a deltacoronavirus species, strain, genotype, subgenotype, serotypes, or genetic/antigenic lineage disclosed herein, in any combination or order thereof. In some embodiments, a broad-spectrum, pan-COV immunogenic composition disclosed herein comprises a nucleotide sequence (e.g., a DNA or RNA) encoding a polypeptide comprising 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more COV peptide antigens, in any combination or order thereof, where each of the 2 or more different COV peptide antigens are from a RNA dependent RNA polymerase from an alphacoronavirus species, strain, genotype, subgenotype, serotypes, or genetic/antigenic lineage disclosed herein, a RNA dependent RNA polymerase from a betacoronavirus species, strain, genotype, subgenotype, serotypes, or genetic/antigenic lineage disclosed herein, a RNA dependent RNA polymerase from a gammacoronavirus species, strain, genotype, subgenotype, serotypes, or genetic/antigenic lineage disclosed herein, a RNA dependent RNA polymerase from a deltacoronavirus species, strain, genotype, subgenotype, serotypes, or genetic/antigenic lineage disclosed herein, a M protein from an alphacoronavirus species, strain, genotype, subgenotype, serotypes, or genetic/antigenic lineage disclosed herein, a M protein from a betacoronavirus species, strain, genotype, subgenotype, serotypes, or genetic/antigenic lineage disclosed herein, a M protein from a gammacoronavirus species, strain, genotype, subgenotype, serotypes, or genetic/antigenic lineage disclosed herein, and/or a M protein from a deltacoronavirus species, strain, genotype, subgenotype, serotypes, or genetic/antigenic lineage disclosed herein, in any combination or order thereof. In some embodiments, a broad-spectrum, pan-COV immunogenic composition disclosed herein comprises a nucleotide sequence (e.g., a DNA or RNA) encoding a polypeptide comprising 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more COV peptide antigens, in any combination or order thereof, where each of the 6 or more different COV peptide antigens are from a RNA dependent RNA polymerase from an alphacoronavirus species, strain, genotype, subgenotype, serotypes, or genetic/antigenic lineage disclosed herein, a RNA dependent RNA polymerase from a betacoronavirus species, strain, genotype, subgenotype, serotypes, or genetic/antigenic lineage disclosed herein, a RNA dependent RNA polymerase from a gammacoronavirus species, strain, genotype, subgenotype, serotypes, or genetic/antigenic lineage disclosed herein, a RNA dependent RNA polymerase from a deltacoronavirus species, strain, genotype, subgenotype, serotypes, or genetic/antigenic lineage disclosed herein, a M protein from an alphacoronavirus species, strain, genotype, subgenotype, serotypes, or genetic/antigenic lineage disclosed herein, a M protein from a betacoronavirus species, strain, genotype, subgenotype, serotypes, or genetic/antigenic lineage disclosed herein, a M protein from a gammacoronavirus species, strain, genotype, subgenotype, serotypes, or genetic/antigenic lineage disclosed herein, and/or a M protein from a deltacoronavirus species, strain, genotype, subgenotype, serotypes, or genetic/antigenic lineage disclosed herein, in any combination or order thereof. In some embodiments, a broad-spectrum, pan-COV immunogenic composition disclosed herein comprises a nucleotide sequence (e.g., a DNA or RNA) encoding a polypeptide comprising 12 or more different COV peptide antigens, such as 13 or more, 14 or more, 15 or more, 16 or more, 17 or more, 18 or more, 19 or more, 20 or more, 21 or more, 22 or more, 23 more, or 24 or more, 26 or more, 28 or more, 30 or more, 32 or more, 34 or more, 36 or more, 38 or more, 40 or more, 42 or more, 44 or more, 46 or more, 48 or more, or 50 or more COV peptide antigens, in any combination or order thereof, where each of the 12 or more different COV peptide antigens are from a RNA dependent RNA polymerase from an alphacoronavirus species, strain, genotype, subgenotype, serotypes, or genetic/antigenic lineage disclosed herein, a RNA dependent RNA polymerase from a betacoronavirus species, strain, genotype, subgenotype, serotypes, or genetic/antigenic lineage disclosed herein, a RNA dependent RNA polymerase from a gammacoronavirus species, strain, genotype, subgenotype, serotypes, or genetic/antigenic lineage disclosed herein, a RNA dependent RNA polymerase from a deltacoronavirus species, strain, genotype, subgenotype, serotypes, or genetic/antigenic lineage disclosed herein, a M protein from an alphacoronavirus species, strain, genotype, subgenotype, serotypes, or genetic/antigenic lineage disclosed herein, a M protein from a betacoronavirus species, strain, genotype, subgenotype, serotypes, or genetic/antigenic lineage disclosed herein, a M protein from a gammacoronavirus species, strain, genotype, subgenotype, serotypes, or genetic/antigenic lineage disclosed herein, and/or a M protein from a deltacoronavirus species, strain, genotype, subgenotype, serotypes, or genetic/antigenic lineage disclosed herein, in any combination or order thereof. In some embodiments, a broad-spectrum, pan-COV immunogenic composition disclosed herein comprises a nucleotide sequence (e.g., a DNA or RNA) encoding a polypeptide comprising 18 or more different COV peptide antigens, such as 19 or more, 20 or more, 21 or more, 22 or more, 23 more, or 24 or more, 26 or more, 28 or more, 30 or more, 32 or more, 34 or more, 36 or more, 38 or more, 40 or more, 42 or more, 44 or more, 46 or more, 48 or more, or 50 or more COV peptide antigens, in any combination or order thereof, where each of the 18 or more different COV peptide antigens are from a RNA dependent RNA polymerase from an alphacoronavirus species, strain, genotype, subgenotype, serotypes, or genetic/antigenic lineage disclosed herein, a RNA dependent RNA polymerase from a betacoronavirus species, strain, genotype, subgenotype, serotypes, or genetic/antigenic lineage disclosed herein, a RNA dependent RNA polymerase from a gammacoronavirus species, strain, genotype, subgenotype, serotypes, or genetic/antigenic lineage disclosed herein, a RNA dependent RNA polymerase from a deltacoronavirus species, strain, genotype, subgenotype, serotypes, or genetic/antigenic lineage disclosed herein, a M protein from an alphacoronavirus species, strain, genotype, subgenotype, serotypes, or genetic/antigenic lineage disclosed herein, a M protein from a betacoronavirus species, strain, genotype, subgenotype, serotypes, or genetic/antigenic lineage disclosed herein, a M protein from a gammacoronavirus species, strain, genotype, subgenotype, serotypes, or genetic/antigenic lineage disclosed herein, and/or a M protein from a deltacoronavirus species, strain, genotype, subgenotype, serotypes, or genetic/antigenic lineage disclosed herein, in any combination or order thereof. In some embodiments, a broad-spectrum, pan-COV immunogenic composition disclosed herein comprises a nucleotide sequence (e.g., a DNA or RNA) encoding a polypeptide comprising 24 or more different COV peptide antigens, such as 26 or more, 28 or more, 30 or more, 32 or more, 34 or more, 36 or more, 38 or more, 40 or more, 42 or more, 44 or more, 46 or more, 48 or more, or 50 or more COV peptide antigens, in any combination or order thereof, where each of the 24 or more different COV peptide antigens are from a RNA dependent RNA polymerase from an alphacoronavirus species, strain, genotype, subgenotype, serotypes, or genetic/antigenic lineage disclosed herein, a RNA dependent RNA polymerase from a betacoronavirus species, strain, genotype, subgenotype, serotypes, or genetic/antigenic lineage disclosed herein, a RNA dependent RNA polymerase from a gammacoronavirus species, strain, genotype, subgenotype, serotypes, or genetic/antigenic lineage disclosed herein, a RNA dependent RNA polymerase from a deltacoronavirus species, strain, genotype, subgenotype, serotypes, or genetic/antigenic lineage disclosed herein, a M protein from an alphacoronavirus species, strain, genotype, subgenotype, serotypes, or genetic/antigenic lineage disclosed herein, a M protein from a betacoronavirus species, strain, genotype, subgenotype, serotypes, or genetic/antigenic lineage disclosed herein, a M protein from a gammacoronavirus species, strain, genotype, subgenotype, serotypes, or genetic/antigenic lineage disclosed herein, and/or a M protein from a deltacoronavirus species, strain, genotype, subgenotype, serotypes, or genetic/antigenic lineage disclosed herein, in any combination or order thereof. In some embodiments, a broad-spectrum, pan-COV immunogenic composition disclosed herein comprises a nucleotide sequence (e.g., a DNA or RNA) encoding a polypeptide comprising 30 or more different COV peptide antigens, such as 32 or more, 34 or more, 36 or more, 38 or more, 40 or more, 42 or more, 44 or more, 46 or more, 48 or more, or 50 or more COV peptide antigens, in any combination or order thereof, where each of the 30 or more different COV peptide antigens are from a RNA dependent RNA polymerase from an alphacoronavirus species, strain, genotype, subgenotype, serotypes, or genetic/antigenic lineage disclosed herein, a RNA dependent RNA polymerase from a betacoronavirus species, strain, genotype, subgenotype, serotypes, or genetic/antigenic lineage disclosed herein, a RNA dependent RNA polymerase from a gammacoronavirus species, strain, genotype, subgenotype, serotypes, or genetic/antigenic lineage disclosed herein, a RNA dependent RNA polymerase from a deltacoronavirus species, strain, genotype, subgenotype, serotypes, or genetic/antigenic lineage disclosed herein, a M protein from an alphacoronavirus species, strain, genotype, subgenotype, serotypes, or genetic/antigenic lineage disclosed herein, a M protein from a betacoronavirus species, strain, genotype, subgenotype, serotypes, or genetic/antigenic lineage disclosed herein, a M protein from a gammacoronavirus species, strain, genotype, subgenotype, serotypes, or genetic/antigenic lineage disclosed herein, and/or a M protein from a deltacoronavirus species, strain, genotype, subgenotype, serotypes, or genetic/antigenic lineage disclosed herein, in any combination or order thereof. In some embodiments, a broad-spectrum, pan-COV immunogenic composition disclosed herein comprises a nucleotide sequence (e.g., a DNA or RNA) encoding a polypeptide comprising 36 or more different COV peptide antigens, such as 38 or more, 40 or more, 42 or more, 44 or more, 46 or more, 48 or more, or 50 or more COV peptide antigens, in any combination or order thereof, where each of the 36 or more different COV peptide antigens are from a RNA dependent RNA polymerase from an alphacoronavirus species, strain, genotype, subgenotype, serotypes, or genetic/antigenic lineage disclosed herein, a RNA dependent RNA polymerase from a betacoronavirus species, strain, genotype, subgenotype, serotypes, or genetic/antigenic lineage disclosed herein, a RNA dependent RNA polymerase from a gammacoronavirus species, strain, genotype, subgenotype, serotypes, or genetic/antigenic lineage disclosed herein, a RNA dependent RNA polymerase from a deltacoronavirus species, strain, genotype, subgenotype, serotypes, or genetic/antigenic lineage disclosed herein, a M protein from an alphacoronavirus species, strain, genotype, subgenotype, serotypes, or genetic/antigenic lineage disclosed herein, a M protein from a betacoronavirus species, strain, genotype, subgenotype, serotypes, or genetic/antigenic lineage disclosed herein, a M protein from a gammacoronavirus species, strain, genotype, subgenotype, serotypes, or genetic/antigenic lineage disclosed herein, and/or a M protein from a deltacoronavirus species, strain, genotype, subgenotype, serotypes, or genetic/antigenic lineage disclosed herein, in any combination or order thereof. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide comprising 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where at least one of the COV peptide antigens is from a RNA dependent RNA polymerase from an alphacoronavirus species, strain, genotype, subgenotype, serotypes, or genetic/antigenic lineage disclosed herein, at least one of the COV peptide antigens is from a RNA dependent RNA polymerase from a betacoronavirus species, strain, genotype, subgenotype, serotypes, or genetic/antigenic lineage disclosed herein, at least one of the COV peptide antigens is from a RNA dependent RNA polymerase from a gammacoronavirus species, strain, genotype, subgenotype, serotypes, or genetic/antigenic lineage disclosed herein, at least one of the COV peptide antigens is from a RNA dependent RNA polymerase from a deltacoronavirus species, strain, genotype, subgenotype, serotypes, or genetic/antigenic lineage disclosed herein, at least one of the COV peptide antigens is from a M protein from an alphacoronavirus species, strain, genotype, subgenotype, serotypes, or genetic/antigenic lineage disclosed herein, at least one of the COV peptide antigens is from a M protein from a betacoronavirus species, strain, genotype, subgenotype, serotypes, or genetic/antigenic lineage disclosed herein, at least one of the COV peptide antigens is from a M protein from a gammacoronavirus species, strain, genotype, subgenotype, serotypes, or genetic/antigenic lineage disclosed herein, and/or at least one of the COV peptide antigens is from a M protein from a deltacoronavirus species, strain, genotype, subgenotype, serotypes, or genetic/antigenic lineage disclosed herein. In some embodiments, a broad-spectrum, pan-COV immunogenic composition disclosed herein comprises a nucleotide sequence (e.g., a DNA or RNA) encoding a polypeptide comprising 12 or more different COV peptide antigens, such as 13 or more, 14 or more, 15 or more, 16 or more, 17 or more, 18 or more, 19 or more, 20 or more, 21 or more, 22 or more, 23 more, or 24 or more, 26 or more, 28 or more, 30 or more, 32 or more, 34 or more, 36 or more, 38 or more, 40 or more, 42 or more, 44 or more, 46 or more, 48 or more, or 50 or more COV peptide antigens, in any combination or order thereof, where at least one of the COV peptide antigens is from a RNA dependent RNA polymerase from an alphacoronavirus species, strain, genotype, subgenotype, serotypes, or genetic/antigenic lineage disclosed herein, at least one of the COV peptide antigens is from a RNA dependent RNA polymerase from a betacoronavirus species, strain, genotype, subgenotype, serotypes, or genetic/antigenic lineage disclosed herein, at least one of the COV peptide antigens is from a RNA dependent RNA polymerase from a gammacoronavirus species, strain, genotype, subgenotype, serotypes, or genetic/antigenic lineage disclosed herein, at least one of the COV peptide antigens is from a RNA dependent RNA polymerase from a deltacoronavirus species, strain, genotype, subgenotype, serotypes, or genetic/antigenic lineage disclosed herein, at least one of the COV peptide antigens is from a M protein from an alphacoronavirus species, strain, genotype, subgenotype, serotypes, or genetic/antigenic lineage disclosed herein, at least one of the COV peptide antigens is from a M protein from a betacoronavirus species, strain, genotype, subgenotype, serotypes, or genetic/antigenic lineage disclosed herein, at least one of the COV peptide antigens is from a M protein from a gammacoronavirus species, strain, genotype, subgenotype, serotypes, or genetic/antigenic lineage disclosed herein, and/or at least one of the COV peptide antigens is from a M protein from a deltacoronavirus species, strain, genotype, subgenotype, serotypes, or genetic/antigenic lineage disclosed herein. In some embodiments, a broad-spectrum, pan-COV immunogenic composition disclosed herein comprises a nucleotide sequence (e.g., a DNA or RNA) encoding a polypeptide comprising 18 or more different COV peptide antigens, such as 19 or more, 20 or more, 21 or more, 22 or more, 23 more, or 24 or more, 26 or more, 28 or more, 30 or more, 32 or more, 34 or more, 36 or more, 38 or more, 40 or more, 42 or more, 44 or more, 46 or more, 48 or more, or 50 or more COV peptide antigens, in any combination or order thereof, where at least one of the COV peptide antigens is from a RNA dependent RNA polymerase from an alphacoronavirus species, strain, genotype, subgenotype, serotypes, or genetic/antigenic lineage disclosed herein, at least one of the COV peptide antigens is from a RNA dependent RNA polymerase from a betacoronavirus species, strain, genotype, subgenotype, serotypes, or genetic/antigenic lineage disclosed herein, at least one of the COV peptide antigens is from a RNA dependent RNA polymerase from a gammacoronavirus species, strain, genotype, subgenotype, serotypes, or genetic/antigenic lineage disclosed herein, at least one of the COV peptide antigens is from a RNA dependent RNA polymerase from a deltacoronavirus species, strain, genotype, subgenotype, serotypes, or genetic/antigenic lineage disclosed herein, at least one of the COV peptide antigens is from a M protein from an alphacoronavirus species, strain, genotype, subgenotype, serotypes, or genetic/antigenic lineage disclosed herein, at least one of the COV peptide antigens is from a M protein from a betacoronavirus species, strain, genotype, subgenotype, serotypes, or genetic/antigenic lineage disclosed herein, at least one of the COV peptide antigens is from a M protein from a gammacoronavirus species, strain, genotype, subgenotype, serotypes, or genetic/antigenic lineage disclosed herein, and/or at least one of the COV peptide antigens is from a M protein from a deltacoronavirus species, strain, genotype, subgenotype, serotypes, or genetic/antigenic lineage disclosed herein. In some embodiments, a broad-spectrum, pan-COV immunogenic composition disclosed herein comprises a nucleotide sequence (e.g., a DNA or RNA) encoding a polypeptide comprising 24 or more different COV peptide antigens, such as 26 or more, 28 or more, 30 or more, 32 or more, 34 or more, 36 or more, 38 or more, 40 or more, 42 or more, 44 or more, 46 or more, 48 or more, or 50 or more COV peptide antigens, in any combination or order thereof, where at least one of the COV peptide antigens is from a RNA dependent RNA polymerase from an alphacoronavirus species, strain, genotype, subgenotype, serotypes, or genetic/antigenic lineage disclosed herein, at least one of the COV peptide antigens is from a RNA dependent RNA polymerase from a betacoronavirus species, strain, genotype, subgenotype, serotypes, or genetic/antigenic lineage disclosed herein, at least one of the COV peptide antigens is from a RNA dependent RNA polymerase from a gammacoronavirus species, strain, genotype, subgenotype, serotypes, or genetic/antigenic lineage disclosed herein, at least one of the COV peptide antigens is from a RNA dependent RNA polymerase from a deltacoronavirus species, strain, genotype, subgenotype, serotypes, or genetic/antigenic lineage disclosed herein, at least one of the COV peptide antigens is from a M protein from an alphacoronavirus species, strain, genotype, subgenotype, serotypes, or genetic/antigenic lineage disclosed herein, at least one of the COV peptide antigens is from a M protein from a betacoronavirus species, strain, genotype, subgenotype, serotypes, or genetic/antigenic lineage disclosed herein, at least one of the COV peptide antigens is from a M protein from a gammacoronavirus species, strain, genotype, subgenotype, serotypes, or genetic/antigenic lineage disclosed herein, and/or at least one of the COV peptide antigens is from a M protein from a deltacoronavirus species, strain, genotype, subgenotype, serotypes, or genetic/antigenic lineage disclosed herein. In some embodiments, a broad-spectrum, pan-COV immunogenic composition disclosed herein comprises a nucleotide sequence (e.g., a DNA or RNA) encoding a polypeptide comprising 30 or more different COV peptide antigens, such as 32 or more, 34 or more, 36 or more, 38 or more, 40 or more, 42 or more, 44 or more, 46 or more, 48 or more, or 50 or more COV peptide antigens, in any combination or order thereof, where at least one of the COV peptide antigens is from a RNA dependent RNA polymerase from an alphacoronavirus species, strain, genotype, subgenotype, serotypes, or genetic/antigenic lineage disclosed herein, at least one of the COV peptide antigens is from a RNA dependent RNA polymerase from a betacoronavirus species, strain, genotype, subgenotype, serotypes, or genetic/antigenic lineage disclosed herein, at least one of the COV peptide antigens is from a RNA dependent RNA polymerase from a gammacoronavirus species, strain, genotype, subgenotype, serotypes, or genetic/antigenic lineage disclosed herein, at least one of the COV peptide antigens is from a RNA dependent RNA polymerase from a deltacoronavirus species, strain, genotype, subgenotype, serotypes, or genetic/antigenic lineage disclosed herein, at least one of the COV peptide antigens is from a M protein from an alphacoronavirus species, strain, genotype, subgenotype, serotypes, or genetic/antigenic lineage disclosed herein, at least one of the COV peptide antigens is from a M protein from a betacoronavirus species, strain, genotype, subgenotype, serotypes, or genetic/antigenic lineage disclosed herein, at least one of the COV peptide antigens is from a M protein from a gammacoronavirus species, strain, genotype, subgenotype, serotypes, or genetic/antigenic lineage disclosed herein, and/or at least one of the COV peptide antigens is from a M protein from a deltacoronavirus species, strain, genotype, subgenotype, serotypes, or genetic/antigenic lineage disclosed herein. In some embodiments, a broad-spectrum, pan-COV immunogenic composition disclosed herein comprises a nucleotide sequence (e.g., a DNA or RNA) encoding a polypeptide comprising 36 or more different COV peptide antigens, such as 38 or more, 40 or more, 42 or more, 44 or more, 46 or more, 48 or more, or 50 or more COV peptide antigens, in any combination or order thereof, where at least one of the COV peptide antigens is from a RNA dependent RNA polymerase from an alphacoronavirus species, strain, genotype, subgenotype, serotypes, or genetic/antigenic lineage disclosed herein, at least one of the COV peptide antigens is from a RNA dependent RNA polymerase from a betacoronavirus species, strain, genotype, subgenotype, serotypes, or genetic/antigenic lineage disclosed herein, at least one of the COV peptide antigens is from a RNA dependent RNA polymerase from a gammacoronavirus species, strain, genotype, subgenotype, serotypes, or genetic/antigenic lineage disclosed herein, at least one of the COV peptide antigens is from a RNA dependent RNA polymerase from a deltacoronavirus species, strain, genotype, subgenotype, serotypes, or genetic/antigenic lineage disclosed herein, at least one of the COV peptide antigens is from a M protein from an alphacoronavirus species, strain, genotype, subgenotype, serotypes, or genetic/antigenic lineage disclosed herein, at least one of the COV peptide antigens is from a M protein from a betacoronavirus species, strain, genotype, subgenotype, serotypes, or genetic/antigenic lineage disclosed herein, at least one of the COV peptide antigens is from a M protein from a gammacoronavirus species, strain, genotype, subgenotype, serotypes, or genetic/antigenic lineage disclosed herein, and/or at least one of the COV peptide antigens is from a M protein from a deltacoronavirus species, strain, genotype, subgenotype, serotypes, or genetic/antigenic lineage disclosed herein. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including one or more different COV peptide antigens, such as, e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the one or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises a nucleotide sequence (e.g., a DNA or RNA) encoding a polypeptide including 2 or more different COV peptide antigens, such as, e.g., 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 2 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises a nucleotide sequence (e.g., a DNA or RNA) encoding a polypeptide including 6 or more different COV peptide antigens, such as, e.g., 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 6 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises a nucleotide sequence (e.g., a DNA or RNA) encoding a polypeptide including 12 or more different COV peptide antigens, such as, e.g., 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 12 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises a nucleotide sequence (e.g., a DNA or RNA) encoding a polypeptide including 18 or more different COV peptide antigens, such as, e.g., 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 18 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises a nucleotide sequence (e.g., a DNA or RNA) encoding a polypeptide including 24 or more different COV peptide antigens, such as, e.g., 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 24 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises a nucleotide sequence (e.g., a DNA or RNA) encoding a polypeptide including 30 or more different COV peptide antigens, such as, e.g., 32 or more, 34 or more, 36 or more, 38 or more, 40 or more, 42 or more, 44 or more, 46 or more, 48 or more, or 50 or more different COV peptide antigens, in any combination or order thereof, where each of the 30 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises a nucleotide sequence (e.g., a DNA or RNA) encoding a polypeptide including 36 or more different COV peptide antigens, such as, e.g., 38 or more, 40 or more, 42 or more, 44 or more, 46 or more, 48 or more, or 50 or more different COV peptide antigens, in any combination or order thereof, where each of the 36 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including one or more different COV peptide antigens, such as, e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the one or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises a nucleotide sequence (e.g., a DNA or RNA) encoding a polypeptide including 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 2 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises a nucleotide sequence (e.g., a DNA or RNA) encoding a polypeptide including 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 6 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises a nucleotide sequence (e.g., a DNA or RNA) encoding a polypeptide including 12 or more different COV peptide antigens, such as 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 12 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises a nucleotide sequence (e.g., a DNA or RNA) encoding a polypeptide including 18 or more different COV peptide antigens, such as 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 18 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises a nucleotide sequence (e.g., a DNA or RNA) encoding a polypeptide including 24 or more different COV peptide antigens, such as, e.g., 26 or more, 28 or more, 30 or more, 32 or more, 34 or more, 36 or more, 38 or more, 40 or more, 42 or more, 44 or more, 46 or more, 48 or more, or 50 or more, in any combination or order thereof, where each of the 24 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises a nucleotide sequence (e.g., a DNA or RNA) encoding a polypeptide including 30 or more different COV peptide antigens, such as, e.g., 32 or more, 34 or more, 36 or more, 38 or more, 40 or more, 42 or more, 44 or more, 46 or more, 48 or more, or 50 or more, in any combination or order thereof, where each of the 30 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises a nucleotide sequence (e.g., a DNA or RNA) encoding a polypeptide including 36 or more different COV peptide antigens, such as, e.g., 38 or more, 40 or more, 42 or more, 44 or more, 46 or more, 48 or more, or 50 or more, in any combination or order thereof, where each of the 36 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises 2 or more nucleotide sequences, such as, 3 or more, 4 or more, 5 or more, 6 or more, 7 or more, 8 or more, 9 or more, or 10 or more nucleotide sequences, each of the 2 or more nucleotide sequences encoding a polypeptide including one or more different COV peptide antigens, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the one or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 2 or more nucleotide sequences, such as, 3 or more, 4 or more, 5 or more, 6 or more, 7 or more, 8 or more, 9 or more, or 10 or more nucleotide sequences, each of the 2 or more nucleotide sequences encoding a polypeptide including 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 2 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 2 or more nucleotide sequences, such as, 3 or more, 4 or more, 5 or more, 6 or more, 7 or more, 8 or more, 9 or more, or 10 or more nucleotide sequences, each of the 2 or more nucleotide sequences encoding a polypeptide including 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 6 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 2 or more nucleotide sequences, such as, 3 or more, 4 or more, 5 or more, 6 or more, 7 or more, 8 or more, 9 or more, or 10 or more nucleotide sequences, each of the 2 or more nucleotide sequences encoding a polypeptide including 12 or more different COV peptide antigens, such as 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 12 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 2 or more nucleotide sequences, such as, 3 or more, 4 or more, 5 or more, 6 or more, 7 or more, 8 or more, 9 or more, or 10 or more nucleotide sequences, each of the 2 or more nucleotide sequences encoding a polypeptide including 18 or more different COV peptide antigens, such as 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 18 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 2 or more nucleotide sequences, such as, 3 or more, 4 or more, 5 or more, 6 or more, 7 or more, 8 or more, 9 or more, or 10 or more nucleotide sequences, each of the 2 or more nucleotide sequences encoding a polypeptide including 24 or more different COV peptide antigens, such as 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 24 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 2 or more nucleotide sequences, such as, 3 or more, 4 or more, 5 or more, 6 or more, 7 or more, 8 or more, 9 or more, or 10 or more nucleotide sequences, each of the 2 or more nucleotide sequences encoding a polypeptide including 30 or more different COV peptide antigens, such as 32 or more, 34 or more, 36 or more, 38 or more, 40 or more, 42 or more, 44 or more, 46 or more, 48 or more, or 50 or more different COV peptide antigens, in any combination or order thereof, where each of the 30 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 2 or more nucleotide sequences, such as, 3 or more, 4 or more, 5 or more, 6 or more, 7 or more, 8 or more, 9 or more, or 10 or more nucleotide sequences, each of the 2 or more nucleotide sequences encoding a polypeptide including 36 or more different COV peptide antigens, such as 38 or more, 40 or more, 42 or more, 44 or more, 46 or more, 48 or more, or 50 or more different COV peptide antigens, in any combination or order thereof, where each of the 36 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises 2 or more nucleotide sequences, such as, 3 or more, 4 or more, 5 or more, 6 or more, 7 or more, 8 or more, 9 or more, or 10 or more nucleotide sequences, each of the 2 or more nucleotide sequences encoding a polypeptide including one or more different COV peptide antigens, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the one or more different COV peptide antigens has the amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 2 or more nucleotide sequences, such as, 3 or more, 4 or more, 5 or more, 6 or more, 7 or more, 8 or more, 9 or more, or 10 or more nucleotide sequences, each of the 2 or more nucleotide sequences encoding a polypeptide including 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 2 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 2 or more nucleotide sequences, such as, 3 or more, 4 or more, 5 or more, 6 or more, 7 or more, 8 or more, 9 or more, or 10 or more nucleotide sequences, each of the 2 or more nucleotide sequences encoding a polypeptide including 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 6 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 2 or more nucleotide sequences, such as, 3 or more, 4 or more, 5 or more, 6 or more, 7 or more, 8 or more, 9 or more, or 10 or more nucleotide sequences, each of the 2 or more nucleotide sequences encoding a polypeptide including 12 or more different COV peptide antigens, such as 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 12 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 2 or more nucleotide sequences, such as, 3 or more, 4 or more, 5 or more, 6 or more, 7 or more, 8 or more, 9 or more, or 10 or more nucleotide sequences, each of the 2 or more nucleotide sequences encoding a polypeptide including 18 or more different COV peptide antigens, such as 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 18 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 2 or more nucleotide sequences, such as, 3 or more, 4 or more, 5 or more, 6 or more, 7 or more, 8 or more, 9 or more, or 10 or more nucleotide sequences, each of the 2 or more nucleotide sequences encoding a polypeptide including 24 or more different COV peptide antigens, such as 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 24 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 2 or more nucleotide sequences, such as, 3 or more, 4 or more, 5 or more, 6 or more, 7 or more, 8 or more, 9 or more, or 10 or more nucleotide sequences, each of the 2 or more nucleotide sequences encoding a polypeptide including 30 or more different COV peptide antigens, such as 32 or more, 34 or more, 36 or more, 38 or more, 40 or more, 42 or more, 44 or more, 46 or more, 48 or more, or 50 or more different COV peptide antigens, in any combination or order thereof, where each of the 30 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, a broad-spectrum, pan-COV immunogenic composition disclosed herein comprises 2 or more nucleotide sequences, such as, 3 or more, 4 or more, 5 or more, 6 or more, 7 or more, 8 or more, 9 or more, or 10 or more nucleotide sequences, each of the 2 or more nucleotide sequences encoding a polypeptide including 36 or more different COV peptide antigens, such as 38 or more, 40 or more, 42 or more, 44 or more, 46 or more, 48 or more, or 50 or more different COV peptide antigens, in any combination or order thereof, where each of the 36 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

A broad-spectrum, pan-COV immunogenic composition disclosed herein may comprises a nucleotide sequence (e.g., DNA or RNA) that further encodes linkers/spacers. In some embodiments, a linkers/spacer separates each of the one or more COV peptide antigens encoded by a nucleotide sequence disclosed herein. In some embodiments, a linkers/spacer separates a subset of the one or more COV peptide antigens encoded by a nucleotide sequence disclosed herein. A linker/spacer disclosed herein is a cleavage site recognized by one or more cellular proteases in a host cell. Non-limiting examples of linkers/spacers disclosed herein include an AAY peptide linker/spacer (SEQ ID NO: 18), GGS peptide linker/spacer (SEQ ID NO: 19), GPGPG peptide linker/spacer (SEQ ID NO: 20), and an EAAAK peptide linker/spacer (SEQ ID NO: 21). However, other peptide linkers/spacers may also be utilized in accordance with the disclosure to space/link the one or more COV peptide antigens encoded by the nucleotide sequence of a broad-spectrum, pan-COV immunogenic composition disclosed herein so long as the peptide linker/spacer is recognized by and cleaved by one or more cellular proteases in a host cell.

In some embodiments, a broad-spectrum, pan-COV immunogenic composition disclosed herein comprises a nucleotide sequence (e.g., a DNA or RNA) encoding a polypeptide including one or more different COV peptide antigens, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the one or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, and where a peptide linker/spacer separates a subset of the one or more different COV peptide antigens. In some embodiments, a broad-spectrum, pan-COV immunogenic composition disclosed herein comprises a nucleotide sequence (e.g., a DNA or RNA) encoding a polypeptide including one or more different COV peptide antigens, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the one or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, and where a peptide linker/spacer separates a subset of the one or more different COV peptide antigens, except for the amino-terminal and carboxyl-terminal ends of the polypeptide. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 2 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, and where a peptide linker/spacer separates a subset of the 2 or more different COV peptide antigens. In some embodiments, a broad-spectrum, pan-COV immunogenic composition disclosed herein comprises a nucleotide sequence (e.g., a DNA or RNA) encoding a polypeptide including 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 2 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, and where a peptide linker/spacer separates a subset of the 2 or more different COV peptide antigens except for the amino-terminal and carboxyl-terminal ends of the polypeptide. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 6 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, and where a peptide linker/spacer separates a subset of the 6 or more different COV peptide antigens. In some embodiments, a broad-spectrum, pan-COV immunogenic composition disclosed herein comprises a nucleotide sequence (e.g., a DNA or RNA) encoding a polypeptide including 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 6 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, and where a peptide linker/spacer separates a subset of the 6 or more different COV peptide antigens except for the amino-terminal and carboxyl-terminal ends of the polypeptide. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 12 or more different COV peptide antigens, such as 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 12 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, and where a peptide linker/spacer separates a subset of the 12 or more different COV peptide antigens. In some embodiments, a broad-spectrum, pan-COV immunogenic composition disclosed herein comprises a nucleotide sequence (e.g., a DNA or RNA) encoding a polypeptide including 12 or more different COV peptide antigens, such as 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 12 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, and where a peptide linker/spacer separates a subset of the 12 or more different COV peptide antigens except for the amino-terminal and carboxyl-terminal ends of the polypeptide. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 18 or more different COV peptide antigens, such as 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 18 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, and where a peptide linker/spacer separates a subset of the 18 or more different COV peptide antigens. In some embodiments, a broad-spectrum, pan-COV immunogenic composition disclosed herein comprises a nucleotide sequence (e.g., a DNA or RNA) encoding a polypeptide including 18 or more different COV peptide antigens, such as 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 18 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, and where a peptide linker/spacer separates a subset of the 18 or more different COV peptide antigens except for the amino-terminal and carboxyl-terminal ends of the polypeptide. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 24 or more different COV peptide antigens, such as, e.g., 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 24 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, and where a peptide linker/spacer separates a subset of the 24 or more different COV peptide antigens. In some embodiments, a broad-spectrum, pan-COV immunogenic composition disclosed herein comprises a nucleotide sequence (e.g., a DNA or RNA) encoding a polypeptide including 24 or more different or more different COV peptide antigens, such as, e.g., 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where the each of the 24 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 24 or more different COV peptide antigens except for the amino-terminal and carboxyl-terminal ends of the polypeptide. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 30 or more different COV peptide antigens, such as, e.g., 32 or more, 34 or more, 36 or more, 38 or more, 40 or more, 42 or more, 44 or more 46 or more, 48 or more, or 50 or more different COV peptide antigens, in any combination or order thereof, where each of the 30 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, and where a peptide linker/spacer separates a subset of the 30 or more different COV peptide antigens. In some embodiments, a broad-spectrum, pan-COV immunogenic composition disclosed herein comprises a nucleotide sequence (e.g., a DNA or RNA) encoding a polypeptide including 30 or more different or more different COV peptide antigens, such as, e.g., 32 or more, 34 or more, 36 or more, 38 or more, 40 or more, 42 or more, 44 or more 46 or more, 48 or more, or 50 or more different COV peptide antigens, in any combination or order thereof, where the each of the 30 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 30 or more different COV peptide antigens except for the amino-terminal and carboxyl-terminal ends of the polypeptide. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 36 or more different COV peptide antigens, such as, e.g., 38 or more, 40 or more, 42 or more, 44 or more 46 or more, 48 or more, or 50 or more, in any combination or order thereof, where each of the 36 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, and where a peptide linker/spacer separates a subset of the 36 or more different COV peptide antigens. In some embodiments, a broad-spectrum, pan-COV immunogenic composition disclosed herein comprises a nucleotide sequence (e.g., a DNA or RNA) encoding a polypeptide including 36 or more different COV peptide antigens such as, e.g., 38 or more, 40 or more, 42 or more, 44 or more 46 or more, 48 or more, or 50 or more, in any combination or order thereof, where the each of the 36 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 36 or more different COV peptide antigens except for the amino-terminal and carboxyl-terminal ends of the polypeptide. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including one or more different COV peptide antigens, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the one or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the one or more different COV peptide antigens. In some embodiments, a broad-spectrum, pan-COV immunogenic composition disclosed herein comprises a nucleotide sequence (e.g., a DNA or RNA) encoding a polypeptide including one or more different COV peptide antigens, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the one or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the one or more different COV peptide antigens except for the amino-terminal and carboxyl-terminal ends of the polypeptide. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order, where each of the 2 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 2 or more different COV peptide antigens. In some embodiments, a broad-spectrum, pan-COV immunogenic composition disclosed herein comprises a nucleotide sequence (e.g., a DNA or RNA) encoding a polypeptide including 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order, where each of the 2 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 2 or more different COV peptide antigens except for the amino-terminal and carboxyl-terminal ends of the polypeptide. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order, where each of the 6 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 6 or more different COV peptide antigens. In some embodiments, a broad-spectrum, pan-COV immunogenic composition disclosed herein comprises a nucleotide sequence (e.g., a DNA or RNA) encoding a polypeptide including 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order, where each of the 6 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 6 or more different COV peptide antigens except for the amino-terminal and carboxyl-terminal ends of the polypeptide. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 12 or more different COV peptide antigens, such as 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order, where each of the 12 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 12 or more different COV peptide antigens. In some embodiments, a broad-spectrum, pan-COV immunogenic composition disclosed herein comprises a nucleotide sequence (e.g., a DNA or RNA) encoding a polypeptide including 12 or more different COV peptide antigens, such as 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order, where each of the 12 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 12 or more different COV peptide antigens except for the amino-terminal and carboxyl-terminal ends of the polypeptide. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 18 or more different COV peptide antigens, such as 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order, where each of the 18 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 18 or more different COV peptide antigens. In some embodiments, a broad-spectrum, pan-COV immunogenic composition disclosed herein comprises a nucleotide sequence (e.g., a DNA or RNA) encoding a polypeptide including 18 or more different COV peptide antigens, such as 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order, where each of the 18 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 18 or more different COV peptide antigens except for the amino-terminal and carboxyl-terminal ends of the polypeptide. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 24 or more different COV antigens, such as, e.g., 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order, where each of the 24 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 24 or more different COV peptide antigens. In some embodiments, a broad-spectrum, pan-COV immunogenic composition disclosed herein comprises a nucleotide sequence (e.g., a DNA or RNA) encoding a polypeptide including 24 or more different COV antigens, such as, e.g., 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 24 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 24 or more different COV peptide antigens except for the amino-terminal and carboxyl-terminal ends of the polypeptide. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 30 or more different COV antigens, such as, e.g., such as, e.g., 32 or more, 34 or more, 36 or more, 38 or more, 40 or more, 42 or more, 44 or more 46 or more, 48 or more, or 50 or more different COV peptide antigens, in any combination or order, where each of the 30 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 30 or more different COV peptide antigens. In some embodiments, a broad-spectrum, pan-COV immunogenic composition disclosed herein comprises a nucleotide sequence (e.g., a DNA or RNA) encoding a polypeptide including 30 or more different COV antigens, such as, e.g., 32 or more, 34 or more, 36 or more, 38 or more, 40 or more, 42 or more, 44 or more 46 or more, 48 or more, or 50 or more different COV peptide antigens, in any combination or order thereof, where each of the 30 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 30 or more different COV peptide antigens except for the amino-terminal and carboxyl-terminal ends of the polypeptide. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 36 or more different COV antigens, such as, e.g., 38 or more, 40 or more, 42 or more, 44 or more 46 or more, 48 or more, or 50 or more, in any combination or order, where each of the 36 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 36 or more different COV peptide antigens. In some embodiments, a broad-spectrum, pan-COV immunogenic composition disclosed herein comprises a nucleotide sequence (e.g., a DNA or RNA) encoding a polypeptide including 36 or more different COV antigens, such as, e.g., 38 or more, 40 or more, 42 or more, 44 or more 46 or more, 48 or more, or 50 or more, in any combination or order thereof, where each of the 36 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 36 or more different COV peptide antigens except for the amino-terminal and carboxyl-terminal ends of the polypeptide. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including one or more different COV peptide antigens, in any combination or order thereof, where each of the one or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the one or more different COV peptide antigens, and where each of the peptide linker/spacers has the amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including one or more different COV peptide antigens, in any combination or order thereof, where each of the one or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the one or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 2 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 2 or more different COV peptide antigens, and where each of the peptide linker/spacers has the amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 2 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 2 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 6 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 6 or more different COV peptide antigens, and where each of the peptide linker/spacers has the amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 6 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 6 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 12 or more different COV peptide antigens, such as 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 12 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 12 or more different COV peptide antigens, and where each of the peptide linker/spacers has the amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 12 or more different COV peptide antigens, such as 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 12 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 12 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 18 or more different COV peptide antigens, such as 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 18 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 18 or more different COV peptide antigens, and where each of the peptide linker/spacers has the amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 18 or more different COV peptide antigens, such as 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 18 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 18 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 24 or more different COV peptide antigens, in any combination or order thereof, the 24 or more different COV peptide antigens, such as, e.g., 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 24 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 24 or more different COV peptide antigens, and where each of the peptide linker/spacers has the amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 24 or more different COV peptide antigens, such as, e.g., 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 24 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 24 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 30 or more different COV peptide antigens, in any combination or order thereof, the 30 or more different COV peptide antigens, such as, e.g., 32 or more, 34 or more, 36 or more, 38 or more, 40 or more, 42 or more, 44 or more 46 or more, 48 or more, or 50 or more different COV peptide antigens, in any combination or order thereof, where each of the 30 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 30 or more different COV peptide antigens, and where each of the peptide linker/spacers has the amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 30 or more different COV peptide antigens, such as, e.g., 32 or more, 34 or more, 36 or more, 38 or more, 40 or more, 42 or more, 44 or more 46 or more, 48 or more, or 50 or more different COV peptide antigens, in any combination or order thereof, where each of the 30 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 30 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 36 or more different COV peptide antigens, such as, e.g., 38 or more, 40 or more, 42 or more, 44 or more 46 or more, 48 or more, or 50 or more different COV peptide antigens, in any combination or order thereof, where each of the 36 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 36 or more different COV peptide antigens, and where each of the peptide linker/spacers has the amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 36 or more different COV peptide antigens, such as, e.g., 38 or more, 40 or more, 42 or more, 44 or more 46 or more, 48 or more, or 50 or more different COV peptide antigens, in any combination or order thereof, where each of the 36 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 36 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including one or more different COV peptide antigens, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the one or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the one or more different COV peptide antigens, and where each of the peptide linker/spacers has the amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including one or more different COV peptide antigens, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the one or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the one or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 2 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 2 or more different COV peptide antigens, and where each of the peptide linker/spacer has the amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 2 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 2 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 6 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 6 or more different COV peptide antigens, and where each of the peptide linker/spacer has the amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 6 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 6 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 12 or more different COV peptide antigens, such as 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 12 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 12 or more different COV peptide antigens, and where each of the peptide linker/spacer has the amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 12 or more different COV peptide antigens, such as 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 12 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 12 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 18 or more different COV peptide antigens, such as 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 18 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 18 or more different COV peptide antigens, and where each of the peptide linker/spacer has the amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 18 or more different COV peptide antigens, such as 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 18 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 18 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 24 or more different COV peptide antigens, such as, e.g., 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 24 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 24 or more different COV peptide antigens, and where each of the peptide linker/spacers has an amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 24 or more different COV peptide antigens, such as, e.g., 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 34 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 24 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 30 or more different COV peptide antigens, such as, e.g., 32 or more, 34 or more, 36 or more, 38 or more, 40 or more, 42 or more, 44 or more 46 or more, 48 or more, or 50 or more different COV peptide antigens, in any combination or order thereof, where each of the 30 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 30 or more different COV peptide antigens, and where each of the peptide linker/spacers has an amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 30 or more different COV peptide antigens, such as, e.g., 32 or more, 34 or more, 36 or more, 38 or more, 40 or more, 42 or more, 44 or more 46 or more, 48 or more, or 50 or more different COV peptide antigens, in any combination or order thereof, where each of the 30 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 30 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 36 or more different COV peptide antigens, such as, e.g., 38 or more, 40 or more, 42 or more, 44 or more 46 or more, 48 or more, or 50 or more different COV peptide antigens, in any combination or order thereof, where each of the 36 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 36 or more different COV peptide antigens, and where each of the peptide linker/spacers has an amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 36 or more different COV peptide antigens, such as, e.g., 38 or more, 40 or more, 42 or more, 44 or more 46 or more, 48 or more, or 50 or more different COV peptide antigens, in any combination or order thereof, where each of the 36 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates a subset of the 36 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including one or more different COV peptide antigens, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the one or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the one or more different COV peptide antigens, and where each of the peptide linker/spacers has the amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including one or more different COV peptide antigens, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the one or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the one or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 2 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 2 or more different COV peptide antigens, and where each of the peptide linker/spacers has an amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 2 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 2 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 6 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 6 or more different COV peptide antigens, and where each of the peptide linker/spacer has the amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 6 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 6 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 12 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 12 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 12 or more different COV peptide antigens, and where each of the peptide linker/spacer has the amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 12 or more different COV peptide antigens, such as 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 12 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 12 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 18 or more different COV peptide antigens, such as 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 18 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 18 or more different COV peptide antigens, and where each of the peptide linker/spacer has the amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 18 or more different COV peptide antigens, such as 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 18 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 18 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 24 or more different COV peptide antigens, such as, e.g., 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 24 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 24 or more different COV peptide antigens, and where each of the peptide linker/spacers has an amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 24 or more different COV peptide antigens, such as, e.g., 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 24 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 24 or more different COV peptide antigens except for the amino-terminal and carboxyl-terminal ends of the polypeptide, and where each of the peptide linker/spacer has an amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 30 or more different COV peptide antigens, such as, e.g., 32 or more, 34 or more, 36 or more, 38 or more, 40 or more, 42 or more, 44 or more 46 or more, 48 or more, or 50 or more different COV peptide antigens, in any combination or order thereof, where each of the 30 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 30 or more different COV peptide antigens, and where each of the peptide linker/spacers has an amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 30 or more different COV peptide antigens, such as, e.g., 32 or more, 34 or more, 36 or more, 38 or more, 40 or more, 42 or more, 44 or more 46 or more, 48 or more, or 50 or more different COV peptide antigens, in any combination or order thereof, where each of the 30 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 30 or more different COV peptide antigens except for the amino-terminal and carboxyl-terminal ends of the polypeptide, and where each of the peptide linker/spacer has an amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 36 or more different COV peptide antigens, such as, e.g., 38 or more, 40 or more, 42 or more, 44 or more 46 or more, 48 or more, or 50 or more different COV peptide antigens, in any combination or order thereof, where each of the 36 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 36 or more different COV peptide antigens, and where each of the peptide linker/spacers has an amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 36 or more different COV peptide antigens, such as, e.g., 38 or more, 40 or more, 42 or more, 44 or more 46 or more, 48 or more, or 50 or more different COV peptide antigens, in any combination or order thereof, where each of the 36 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 36 or more different COV peptide antigens except for the amino-terminal and carboxyl-terminal ends of the polypeptide, and where each of the peptide linker/spacer has an amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including one or more different COV peptide antigens, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the one or more different COV peptide antigens has an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the one or more different COV peptide antigens, and where each of the peptide linker/spacers has an amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including one or more different COV peptide antigens, such as 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the one or more different COV peptide antigens has an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the one or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 2 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 2 or more different COV peptide antigens, and where each of the peptide linker/spacers has an amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 2 or more different COV peptide antigens, such as 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 2 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 2 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 6 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 6 or more different COV peptide antigens, and where each of the peptide linker/spacers has an amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 6 or more different COV peptide antigens, such as 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 6 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 6 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 12 or more different COV peptide antigens, such as 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 12 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 12 or more different COV peptide antigens, and where each of the peptide linker/spacers has an amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 12 or more different COV peptide antigens, such as 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 12 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 12 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 18 or more different COV peptide antigens, such as 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 18 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 18 or more different COV peptide antigens, and where each of the peptide linker/spacers has an amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 18 or more different COV peptide antigens, such as 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 18 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 18 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 24 or more different COV peptide antigens, such as, e.g., 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 24 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 24 or more different COV peptide antigens, and where each of the peptide linker/spacers has an amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 24 or more different COV peptide antigens, such as, e.g., 26 or more, 28 or more, 30 or more, 32 or more, 34 or more, 36 or more, 38 or more, 40 or more, 42 or more, 44 or more, 46 or more, 48 or more, or 50 or more different COV peptide antigens, in any combination or order thereof, where each of the 24 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 24 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 30 or more different COV peptide antigens, such as, e.g., 32 or more, 34 or more, 36 or more, 38 or more, 40 or more, 42 or more, 44 or more 46 or more, 48 or more, or 50 or more different COV peptide antigens, in any combination or order thereof, where each of the 30 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 30 or more different COV peptide antigens, and where each of the peptide linker/spacers has an amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 30 or more different COV peptide antigens, such as, e.g., 32 or more, 34 or more, 36 or more, 38 or more, 40 or more, 42 or more, 44 or more 46 or more, 48 or more, or 50 or more different COV peptide antigens, in any combination or order thereof, where each of the 30 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 30 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide including 36 or more different COV peptide antigens, such as, e.g., 38 or more, 40 or more, 42 or more, 44 or more 46 or more, 48 or more, or 50 or more different COV peptide antigens, in any combination or order thereof, where each of the 36 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 36 or more different COV peptide antigens, and where each of the peptide linker/spacers has an amino acid sequence of SEQ ID NO: 18, SEQ ID NO: 19, SEQ ID NO: 20, or SEQ ID NO: 21. In some embodiments, a broad-spectrum, pan-COV immunogenic composition disclosed herein comprises a nucleotide sequence (e.g., a DNA or RNA) encoding a polypeptide including 36 or more different COV peptide antigens, such as, e.g., 38 or more, 40 or more, 42 or more, 44 or more 46 or more, 48 or more, or 50 or more different COV peptide antigens, in any combination or order thereof, where each of the 36 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, where a peptide linker/spacer separates each of the 36 or more different COV peptide antigens except for the amino-terminal and carboxyl-terminal ends of the polypeptide, and where each of the peptide linker/spacers has the amino acid sequence of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells inducing cytotoxicity. Some antibodies may also have viral neutralizing activity.

In some embodiments, a broad-spectrum, pan-COV immunogenic composition disclosed herein comprises a nucleotide sequence (e.g., a DNA or RNA) encoding a polypeptide of Polypeptide Structure I:

COVPA¹-(PLS¹ₘ)-[COVPA²-(PLS²ₘ)]ₙ-COVPA³ Polypeptide Structure I

wherein COVPA¹ is a first COV peptide antigen disclosed herein, COVPA² is a second COV peptide antigen disclosed herein, COVPA³ is a third COV peptide antigen disclosed herein, PLS¹ and PLS² are each independently a peptide linker/spacer, m is independently any integer from 0 to 1, n is any integer from 0 to 52, 2 to 48, 4 to 46, 6 to 44, 8 to 42, 10 to 40, 12 to 38, 14 to 36, 16 to 34, 18 to 32, or 20 to 30, wherein the polypeptide of Polypeptide Structure I comprises at least two peptide antigens selected from COVPA¹, COVPA², and COVPA³, or any combination thereof, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In aspects of the above embodiments, the polypeptide of Polypeptide Structure I comprises at least 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, or 52 peptide antigens, wherein for each n, COVPA² can be a different peptide antigen or the same peptide antigen, wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen disclosed herein, COVPA² is a second COV peptide antigen disclosed herein, COVPA³ is a third COV peptide antigen disclosed herein, PLS¹ and PLS² are each independently a peptide linker/spacer, m is independently any integer from 0 to 1, n is any integer from 0 to 4, 4, to 8, 8 to 12, 12 to 16, 16 to 20, 20 to 24, 24 to 28, 28 to 32, 32 to 36, 36 to 40, 40 to 44, 44 to 48, or 48 to 52, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In other aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen disclosed herein, COVPA² is a second COV peptide antigen disclosed herein, COVPA³ is a third COV peptide antigen disclosed herein, PLS¹ and PLS² are each independently a peptide linker/spacer, m is independently any integer from 0 to 1, n is any integer from 0 to 10, 5 to 15, 10 to 20, 25 to 35, 30 to 40, 35 to 45, or 40 to 50, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In yet other aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen disclosed herein, COVPA² is a second COV peptide antigen disclosed herein, COVPA³ is a third COV peptide antigen disclosed herein, PLS¹ and PLS² are each independently a peptide linker/spacer, m is independently any integer from 0 to 1, n is any integer from 0 to 10, 0 to 20, 0 to 30, 0 to 40, 0 to 50, 5 to 10, 5 to 20, 5 to 30, 5 to 40, 5 to 50, 10 to 20, 10 to 30, 10 to 40, 10 to 50, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages.

In aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer, m is independently any integer from 0 to 1, n is any integer from 0 to 52, 2 to 48, 4 to 46, 6 to 44, 8 to 42, 10 to 40, 12 to 38, 14 to 36, 16 to 34, 18 to 32, or 20 to 30, wherein the polypeptide of Polypeptide Structure I comprises at least two peptide antigens selected from COVPA1, COVPA2, and COVPA3, or any combination thereof, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer, m is independently any integer from 0 to 1, n is any integer from 0 to 4, 4, to 8, 8 to 12, 12 to 16, 16 to 20, 20 to 24, 24 to 28, 28 to 32, 32 to 36, 36 to 40, 40 to 44, 44 to 48, or 48 to 52, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer, m is independently any integer from 0 to 1, n is any integer from 0 to 10, 5 to 15, 10 to 20, 25 to 35, 30 to 40, 35 to 45, or 40 to 50, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer, m is independently any integer from 0 to 1, n is any integer from 0 to 10, 0 to 20, 0 to 30, 0 to 40, 0 to 50, 5 to 10, 5 to 20, 5 to 30, 5 to 40, 5 to 50, 10 to 20, 10 to 30, 10 to 40, 10 to 50, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages.

In aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer of SEQ ID NO: 18, SEQ ID NO: 19, SEQ ID NO: 20, or SEQ ID NO: 21, m is independently any integer from 0 to 1, n is any integer from 0 to 52, 2 to 48, 4 to 46, 6 to 44, 8 to 42, 10 to 40, 12 to 38, 14 to 36, 16 to 34, 18 to 32, or 20 to 30, wherein the polypeptide of Polypeptide Structure I comprises at least two peptide antigens selected from COVPA¹, COVPA², and COVPA³, or any combination thereof, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer of SEQ ID NO: 18, SEQ ID NO: 19, SEQ ID NO: 20, or SEQ ID NO: 21, m is independently any integer from 0 to 1, n is any integer from 0 to 4, 4, to 8, 8 to 12, 12 to 16, 16 to 20, 20 to 24, 24 to 28, 28 to 32, 32 to 36, 36 to 40, 40 to 44, 44 to 48, or 48 to 52, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer of SEQ ID NO: 18, SEQ ID NO: 19, SEQ ID NO: 20, or SEQ ID NO: 21, m is independently any integer from 0 to 1, n is any integer from 0 to 10, 5 to 15, 10 to 20, 25 to 35, 30 to 40, 35 to 45, or 40 to 50, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer of SEQ ID NO: 18, SEQ ID NO: 19, SEQ ID NO: 20, or SEQ ID NO: 21, m is independently any integer from 0 to 1, n is any integer from 0 to 10, 0 to 20, 0 to 30, 0 to 40, 0 to 50, 5 to 10, 5 to 20, 5 to 30, 5 to 40, 5 to 50, 10 to 20, 10 to 30, 10 to 40, 10 to 50, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages.

In aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer, m is independently any integer from 0 to 1, n is any integer from 0 to 52, 2 to 48, 4 to 46, 6 to 44, 8 to 42, 10 to 40, 12 to 38, 14 to 36, 16 to 34, 18 to 32, or 20 to 30, wherein the polypeptide of Polypeptide Structure I comprises at least two peptide antigens selected from COVPA1, COVPA2, and COVPA3, or any combination thereof, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer, m is independently any integer from 0 to 1, n is any integer from 0 to 4, 4, to 8, 8 to 12, 12 to 16, 16 to 20, 20 to 24, 24 to 28, 28 to 32, 32 to 36, 36 to 40, 40 to 44, 44 to 48, or 48 to 52, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer, m is independently any integer from 0 to 1, n is any integer from 0 to 10, 5 to 15, 10 to 20, 25 to 35, 30 to 40, 35 to 45, or 40 to 50, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer, m is independently any integer from 0 to 1, n is any integer from 0 to 10, 0 to 20, 0 to 30, 0 to 40, 0 to 50, 5 to 10, 5 to 20, 5 to 30, 5 to 40, 5 to 50, 10 to 20, 10 to 30, 10 to 40, 10 to 50, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages.

In other aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer of SEQ ID NO: 18, SEQ ID NO: 19, SEQ ID NO: 20, or SEQ ID NO: 21, m is independently any integer from 0 to 1, n is any integer from 0 to 52, 2 to 48, 4 to 46, 6 to 44, 8 to 42, 10 to 40, 12 to 38, 14 to 36, 16 to 34, 18 to 32, or 20 to 30, wherein the polypeptide of Polypeptide Structure I comprises at least two peptide antigens selected from COVPA1, COVPA2, and COVPA3, or any combination thereof, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In other aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer of SEQ ID NO: 18, SEQ ID NO: 19, SEQ ID NO: 20, or SEQ ID NO: 21, m is independently any integer from 0 to 1, n is any integer from 0 to 4, 4, to 8, 8 to 12, 12 to 16, 16 to 20, 20 to 24, 24 to 28, 28 to 32, 32 to 36, 36 to 40, 40 to 44, 44 to 48, or 48 to 52, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In other aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer of SEQ ID NO: 18, SEQ ID NO: 19, SEQ ID NO: 20, or SEQ ID NO: 21, m is independently any integer from 0 to 1, n is any integer from 0 to 10, 5 to 15, 10 to 20, 25 to 35, 30 to 40, 35 to 45, or 40 to 50, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In other aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer of SEQ ID NO: 18, SEQ ID NO: 19, SEQ ID NO: 20, or SEQ ID NO: 21, m is independently any integer from 0 to 1, n is any integer from 0 to 10, 0 to 20, 0 to 30, 0 to 40, 0 to 50, 5 to 10, 5 to 20, 5 to 30, 5 to 40, 5 to 50, 10 to 20, 10 to 30, 10 to 40, 10 to 50, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages.

In aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of SEQ ID NO: 1, COVPA² is a second COV peptide antigen selected from a COV peptide antigen having an amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11, COVPA³ is a third COV peptide antigen selected from a COV peptide antigen having an amino acid sequence of SEQ ID NO: 12, PLS¹ and PLS² are each independently a peptide linker/spacer of SEQ ID NO: 18, SEQ ID NO: 19, SEQ ID NO: 20, or SEQ ID NO: 21, m is independently any integer from 0 to 1, n is any integer from 0 to 52, 2 to 48, 4 to 46, 6 to 44, 8 to 42, 10 to 40, 12 to 38, 14 to 36, 16 to 34, 18 to 32, or 20 to 30, and wherein the polypeptide of Polypeptide Structure I comprises at least two peptide antigens selected from COVPA1, COVPA2, and COVPA3, or any combination thereof, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of SEQ ID NO: 1, COVPA² is a second COV peptide antigen selected from a COV peptide antigen having an amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11, COVPA³ is a third COV peptide antigen selected from a COV peptide antigen having an amino acid sequence of SEQ ID NO: 12, PLS¹ and PLS² are each independently a peptide linker/spacer of SEQ ID NO: 18, SEQ ID NO: 19, SEQ ID NO: 20, or SEQ ID NO: 21, m is independently any integer from 0 to 1, n is any integer from 0 to 4, 4, to 8, 8 to 12, 12 to 16, 16 to 20, 20 to 24, 24 to 28, 28 to 32, 32 to 36, 36 to 40, 40 to 44, 44 to 48, or 48 to 52, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of SEQ ID NO: 1, COVPA² is a second COV peptide antigen selected from a COV peptide antigen having an amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11, COVPA³ is a third COV peptide antigen selected from a COV peptide antigen having an amino acid sequence of SEQ ID NO: 12, PLS¹ and PLS² are each independently a peptide linker/spacer of SEQ ID NO: 18, SEQ ID NO: 19, SEQ ID NO: 20, or SEQ ID NO: 21, m is independently any integer from 0 to 1, n is any integer from 0 to 10, 5 to 15, 10 to 20, 25 to 35, 30 to 40, 35 to 45, or 40 to 50, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In aspects of these embodiments, a broad-spectrum, pan-COV immunogenic composition disclosed herein comprises a nucleotide sequence (e.g., a DNA or RNA) encoding a polypeptide of Polypeptide Structure I, wherein COVPA¹ is a first COV peptide antigen selected from a COV peptide antigen having an amino acid sequence of SEQ ID NO: 1, COVPA² is a second COV peptide antigen selected from a COV peptide antigen having an amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11, COVPA³ is a third COV peptide antigen selected from a COV peptide antigen having an amino acid sequence of SEQ ID NO: 12, PLS¹ and PLS² are each independently a peptide linker/spacer of SEQ ID NO: 18, SEQ ID NO: 19, SEQ ID NO: 20, or SEQ ID NO: 21, m is independently any integer from 0 to 1, n is any integer from 0 to 10, 0 to 20, 0 to 30, 0 to 40, 0 to 50, 5 to 10, 5 to 20, 5 to 30, 5 to 40, 5 to 50, 10 to 20, 10 to 30, 10 to 40, 10 to 50, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages.

In some embodiments, a broad-spectrum, pan-COV immunogenic composition disclosed herein comprises a nucleotide sequence (e.g., a DNA or RNA) encoding a polypeptide of Polypeptide Structure II:

SEQ ID NO: 1—(PLS¹ₘ)—SEQ ID NO: 2—(PLS²ₘ)—SEQ ID NO: 3—(PLS³ₘ)—SEQ ID NO: 4—(PLS⁴ₘ)—SEQ ID NO: 5—(PLS⁵ₘ)—SEQ ID NO: 6—(PLS⁶ₘ)—SEQ ID NO: 7—(PLS⁷ₘ)—SEQ ID NO: 8—(PLS⁸ₘ)—SEQ ID NO: 9—(PLS⁹ₘ)—SEQ ID NO: 10—(PLS¹⁰ₘ)—SEQ ID NO: 11—(PLS¹¹ₘ)—SEQ ID NO: 12 Polypeptide Structure II

wherein PLS¹, PLS², PLS³, PLS⁴, PLS⁵, PLS⁶, PLS⁷, PLS⁸, PLS⁹, PLS¹⁰, and PLS¹¹ are each independently a peptide linker/spacer of SEQ ID NO: 18, SEQ ID NO: 19, SEQ ID NO: 20, or SEQ ID NO: 21, and m is independently any integer from 0 to 1. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages.

In some embodiments, a broad-spectrum, pan-COV immunogenic composition disclosed herein comprises a nucleotide sequence (e.g., a DNA or RNA) encoding a polypeptide of Polypeptide Structure III:

SEQ ID NO: 1—PLS¹—SEQ ID NO: 2—PLS²—SEQ ID NO: 3—PLS³—SEQ ID NO: 4—PLS⁴—SEQ ID NO: 5—PLS⁵—SEQ ID NO: 6—PLS⁶—SEQ ID NO: 7—PLS⁷—SEQ ID NO: 8—PLS⁸—SEQ ID NO: 9—PLS⁹—SEQ ID NO: 10—PLS¹⁰—SEQ ID NO: 11—PLS¹¹—SEQ ID NO: 12 Polypeptide Structure III

wherein PLS¹, PLS², PLS³, PLS⁴, PLS⁵, PLS⁶, PLS⁷, PLS⁸, PLS⁹, PLS¹⁰, and PLS¹¹ are each independently a peptide linker/spacer of SEQ ID NO: 18, SEQ ID NO: 19, SEQ ID NO: 20, or SEQ ID NO: 21. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages.

In some embodiments, a broad-spectrum, pan-COV immunogenic composition disclosed herein comprises a nucleotide sequence (e.g., a DNA or RNA) encoding a polypeptide including the amino acid sequence of SEQ ID NO: 31 (Table 4). In some embodiments, a broad-spectrum, pan-COV immunogenic composition disclosed herein comprises a nucleotide sequence (e.g., a DNA or RNA) encoding a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence of SEQ ID NO: 31. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages.

TABLE 4
Polypeptide Comprising Multiple COV Peptide
Antigens

| SEQ ID | |
|---|---|
| NO: | Amino Acid Sequence of Peptide |
| 31 | TKRNVLPTMTQLNLKYAISGKERARTVAAYTKRNVIPTITQM |
| | NLKYAISAKNRARTVAAYTKRNVLPTLTQMNLKYAISAKNRA |
| | RTVAAYPTGVTLTLLSGNLYAEGFKAAYPTGVTLTLLSGTLL |
| | VEGYKAAYPTGITVTLLSGVLYVDGHRAAYPTGVTLTLLSGV |
| | LLVDGHKAAYPTGITLTILSGTLFFDGIRIAAAYYFIASFRL |
| | FARTRSMWSFNPETNAAYYFVNSIRLFIRTGSWWSFNPETNA |
| | AYGYWIQSIRLFKRCRSWWSFNPESNAAYKLILLWLLQPFTL |
| | VVTIW |

In some embodiments, a broad-spectrum, pan-COV immunogenic composition disclosed herein comprises a DNA nucleotide sequence encoding a polypeptide including the amino acid sequence of SEQ ID NO: 32 (Table 5). In some embodiments, a broad-spectrum, pan-COV immunogenic composition disclosed herein comprises a DNA nucleotide sequence encoding a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence of SEQ ID NO: 32. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages.

TABLE 5
Polypeptide Comprising Multiple COV Peptide
Antigens

| SEQ ID | |
|---|---|
| NO: | Amino Acid Sequence of Peptide |
| 32 | TKRNVLPTMTQLNLKYAISGKERARTVAAYTKRNVIPTITQM |
| | NLKYAISAKNRARTVAAYTKRNVLPTLTQMNLKYAISAKNRA |
| | RTVAAYPTGVTLTLLSGNLYAEGFKAAYPTGVTLTLLSGTLL |
| | VEGYKAAYPTGITVTLLSGVLYVDGHRAAYPTGVTLTLLSGV |
| | LLVDGHKAAYPTGITLTILSGTLFFDGIRIAAAYYFIASFRL |
| | FARTRSMWSFNPETNAAYYFVNSIRLFIRTGSWWSFNPETNA |
| | AYGYWIQSIRLFKRCRSWWSFNPESNAAYKLILLWLLQPFTL |
| | VVTIWAAYHHHHHH |

In some embodiments, a nucleotide sequence disclosed herein may optionally comprise a coding sequence for a stabilizing peptide, the stabilizing peptide sequence being operably linked to and is in frame with the coding sequence of a polypeptide disclosed herein. Stabilizing peptides enhance stability of a polypeptide disclosed herein once the polypeptide is synthesized in the cytosol or to ensure proper processing of a polypeptide disclosed herein and presentation by MHC-I and MHC-II. Non-limiting examples of a stabilizing peptide include ubiquitin, lysosomal-associated membrane protein 1 (LAMP-1), LAMP-2, and LAMP-3 (also known as DC-LAMP). A LAMP like LAMP-1, LAMP-2, or LAMP-3 has an amino to carboxyl organization of a signal peptide, a first luminal domain, a hinge domain, a second luminal domain, and a transmembrane domain. An exemplary sequence for LAMP-1 is SEQ ID NO: 13, for LAMP-2 is SEQ ID NO: 14, and for LAMP-3 is SEQ ID NO: 15. The entire polypeptide for a LAMP need not be used to stabilize a polypeptide disclosed herein as LAMP fragments containing some of the domains function to promote transport of peptides and polypeptides to the endosome. For example, one or more of a first luminal domain, a second luminal domain, a hinge domain, a transmembrane domain, portions thereof, or any combination thereof may be operably linked to and in frame with the coding sequence of a polypeptide disclosed herein. In addition, hybrid molecules are also envisioned such the signal peptide from a LAMP-1, the transmembrane domain from a LAMP-3, and one or more of the first and second luminal domains and hinge domain from a LAMP-3. In some embodiments, a stabilizing peptide is operably linked to the amino-terminus of a polypeptide disclosed herein. In some embodiments, a stabilizing peptide is operably linked to the carboxy-terminus of a polypeptide disclosed herein. In some embodiments, a polypeptide disclosed herein is inserted within a stabilizing peptide disclosed herein.

In some embodiments, a broad-spectrum, pan-COV immunogenic composition disclosed herein comprises a nucleotide sequence (e.g., a DNA or RNA) encoding a polypeptide including one or more COV peptide antigens disclosed herein inserted within a LAMP like LAMP-1. LAMP-2, or LAMP-3, or a LAMP fragment comprising one or more of a first luminal domain, a second luminal domain, a hinge domain, a transmembrane domain, portions thereof, or any combination thereof. In some embodiments, a polypeptide including one or more COV peptide antigens disclosed herein is inserted in between the second luminal domain and the transmembrane domain of a LAMP like LAMP-1, LAMP-2 or LAMP-3, or a LAMP fragment like LAMP-1 fragment, LAMP-2 fragment or LAMP-3 fragment comprising a second luminal domain and a transmembrane domain. In some embodiments, a polypeptide is any one of Polypeptide Structures I-III disclosed herein and the polypeptide is inserted in between the second luminal domain and the transmembrane domain of a LAMP like LAMP-1, LAMP-2 or LAMP-3, or a LAMP fragment like LAMP-1 fragment, LAMP-2 fragment or LAMP-3 fragment comprising a second luminal domain and a transmembrane domain. In some embodiments, a polypeptide is any one of Polypeptide Structures I-III disclosed herein and the polypeptide is inserted in between the second luminal domain and the transmembrane domain of a LAMP-1 protein having the amino acid sequence of SEQ ID NO: 13, a LAMP-2 protein having the amino acid sequence of SEQ ID NO: 14, or a LAMP-3 protein having the amino acid sequence of SEQ ID NO: 15, or a LAMP fragment comprising a second luminal domain and a transmembrane domain of SEQ ID NO: 13, SEQ ID NO: 14, or SEQ ID NO: 15.

In some embodiments, a broad-spectrum, pan-COV immunogenic composition disclosed herein comprises a nucleotide sequence (e.g., a DNA or RNA) encoding a first polypeptide including one or more COV peptide antigens disclosed herein, including any of the polypeptide structures disclosed herein, and a second polypeptide including one or more COV peptide antigens disclosed herein, including any of the polypeptide structures disclosed, inserted within a LAMP like LAMP-1. LAMP-2, or LAMP-3, or a LAMP fragment comprising one or more of a first luminal domain, a second luminal domain, a hinge domain, a transmembrane domain, portions thereof, or any combination thereof. In some embodiments, a second polypeptide including one or more COV peptide antigens disclosed herein is inserted in between the second luminal domain and the transmembrane domain of a LAMP like LAMP-1, LAMP-2 or LAMP-3, or a LAMP fragment like LAMP-1 fragment, LAMP-2 fragment or LAMP-3 fragment comprising a second luminal domain and a transmembrane domain. In some embodiments, a second polypeptide is any one of Polypeptide Structures I-III disclosed herein and the second polypeptide is inserted in between the second luminal domain and the transmembrane domain of a LAMP like LAMP-1, LAMP-2 or LAMP-3, or a LAMP fragment like LAMP-1 fragment, LAMP-2 fragment or LAMP-3 fragment comprising a second luminal domain and a transmembrane domain. In some embodiments, a second polypeptide is any one of Polypeptide Structures I-III disclosed herein and the second polypeptide is inserted in between the second luminal domain and the transmembrane domain of a LAMP-1 having the amino acid sequence of SEQ ID NO: 13, a LAMP-2 protein having the amino acid sequence of SEQ ID NO: 14, or a LAMP-3 protein having the amino acid sequence of SEQ ID NO: 15, or a LAMP fragment comprising a second luminal domain and a transmembrane domain of SEQ ID NO: 13, SEQ ID NO: 14, or SEQ ID NO: 15.

In some embodiments, a broad-spectrum, pan-COV immunogenic composition disclosed herein comprises a nucleotide sequence (e.g., a DNA or RNA) encoding a polypeptide including the amino acid sequence of SEQ ID NO: 33 (Table 6). In some embodiments, a broad-spectrum, pan-COV immunogenic composition disclosed herein comprises a nucleotide sequence (e.g., a DNA or RNA) encoding a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence of SEQ ID NO: 33. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages.

TABLE 6
Polypeptide Comprising Multiple COV Peptide Antigens

| SEQ ID NO: | Amino Acid Sequence of Peptide |
|---|---|
| 33 | TKRNVLPTMTQLNLKYAISGKERARTVAAYTKRNVIPTITQMNLKYAISAKNRARTVAAYTK |
| | RNVLPTLTQMNLKYAISAKNRARTVAAYPTGVTLTLLSGNLYAEGFKAAYPTGVTLTLLSG |
| | TLLVEGYKAAYPTGITVTLLSGVLYVDGHRAAYPTGVTLTLLSGVLLVDGHKAAYPTGITLTI |
| | LSGTLFFDGIRIAAAYYFIASFRLFARTRSMWSFNPETNAAYYFVNSIRLFIRTGSWWSFNP |
| | ETNAAYGYWIQSIRLFKRCRSWWSFNPESNAAYKLILLWLLQPFTLVVTIWAAYHHHHHH |
| | GSGEGRGSLLTCGDVEENPGPYPYDVPDYAMAAPGSARRPLLLLLLLLLLGLMHCASAA |
| | MFMVKNGNGTACIMANFSAAFSVNYDTKSGPKNMTFDLPSDATVVLNRSSCGKENTSDP |
| | SLVIAFGRGHTLTLNFTRNATRYSVQLMSFVYNLSDTHLFPNASSKEIKTVESITDIRADIDK |
| | KYRCVSGTQVHMNNVTVTLHDATIQAYLSNSSFSRGETRCEQDRPSPTTAPPAPPSPSPS |
| | PVPKSPSVDKYNVSGTNGTCLLASMGLQLNLTYERKDNTTVTRLLNINPNKTSASGSCGA |
| | HLVTLELHSEGTTVLLFQFGMNASSSRFFLQGIQLNTILPDARDPAFKAANGSLRALQATV |
| | GNSYKCNAEEHVRVTKAFSVNIFKVWVQAFKVEGGQFGSVEECLLDENSMTKRNVLPTM |
| | TQLNLKYAISGKERARTVAAYTKRNVIPTITQMNLKYAISAKNRARTVAAYTKRNVLPTLTQ |
| | MNLKYAISAKNRARTVAAYPTGVTLTLLSGNLYAEGFKAAYPTGVTLTLLSGTLLVEGYKA |
| | AYPTGITVTLLSGVLYVDGHRAAYPTGVTLTLLSGVLLVDGHKAAYPTGITLTILSGTLFFDG |
| | IRIAAAYYFIASFRLFARTRSMWSFNPETNAAYYFVNSIRLFIRTGSWWSFNPETNAAYGY |
| | WIQSIRLFKRCRSWWSFNPESNAAYKLILLWLLQPFTLVVTIWAAYSMLIPIAVGGALAGLV |
| | LIVLIAYLVGRKRSHAGYQTI |

A broad-spectrum, pan-COV immunogenic composition disclosed herein can comprise a nucleotide sequence (e.g., DNA or RNA) including multiple open reading frames each encoding a polypeptide disclosed herein, such as, Polypeptide Structure I, Polypeptide Structure II, Polypeptide Structure III, or a polypeptide including one or more COV peptide antigens disclosed herein inserted in a LAMP disclosed herein. In some embodiments, a self-cleaving peptide separates each open-reading frame of a RNA (e.g., mRNA) nucleotide sequence molecule disclosed herein containing multiple open-reading frames. In some embodiments, a self-cleaving peptide separates each of the one or more COV peptide antigens encoded by a RNA (e.g., mRNA) nucleotide sequence disclosed herein. In some embodiments, a self-cleaving peptide separates a subset of the one or more COV peptide antigens encoded by a RNA (e.g., mRNA) nucleotide sequence disclosed herein. A self-cleaving peptide disclosed herein can induce ribosomal skipping during translation of a protein. Thus, when inserted in between each of the one or more open reading frames and/or the one or more COV peptide antigens encoded by the RNA (e.g., mRNA) nucleotide sequence, a self-cleaving peptide disclosed herein is used to facilitate synthesis of each COV peptide antigen separately from the larger polypeptide disclosed herein. Non-limiting examples of a self-cleaving peptide include 2A self-cleaving peptides that share a core sequence motif of DXEXNPGP (SEQ ID NO: 22), where X is any amino acid, which causes a ribosome to fail to synthesize a peptide bond. Non-limiting examples of 2A self-cleaving peptides disclosed herein include a T2A self-cleaving peptide EGRGSLLTCGDVEENPGP (SEQ ID NO: 23), a P2A self-cleaving peptide ATNFSLLKQAGDVEENPGP (SEQ ID NO: 24), a E2A self-cleaving peptide QCTNYALLKLAGDVESNPGP (SEQ ID NO: 25), and a F2A self-cleaving peptide VKQTLNFDLLKLAGDVESNPGP (SEQ ID NO: 26). In some embodiments, an optional GSG linker can be added to the N-terminal of a 2A self-cleaving peptide disclosed herein to enhance efficiency of the 2A self-cleaving peptide. Non-limiting examples of 2A self-cleaving peptides disclosed herein comprising a GSG linker include a T2A self-cleaving peptide GSGEGRGSLLTCGDVEENPGP (SEQ ID NO: 27), a P2A self-cleaving peptide GSGATNFSLLKQAGDVEENPGP (SEQ ID NO: 28), a E2A self-cleaving peptide GSGQCTNYALLKLAGDVESNPGP (SEQ ID NO: 29), and a F2A self-cleaving peptide GSGVKQTLNFDLLKLAGDVESNPGP (SEQ ID NO: 30).

In some embodiments, a broad-spectrum, pan-COV immunogenic composition disclosed herein comprises a nucleotide sequence (e.g., a DNA or RNA) encoding a polypeptide of Polypeptide Structure IV:

PA¹-(SCP¹)[PA²-(SCP²)]ₒ-PA³ Polypeptide Structure IV

wherein PA¹ is a first polypeptide comprising one or more COV peptide antigens disclosed herein, such as, e.g., Polypeptide Structure I, II, or III disclosed herein, PA² is a second polypeptide comprising one or more COV peptide antigens disclosed herein, such as, e.g., Polypeptide Structure I, II, or III disclosed herein, PA³ is a third polypeptide comprising one or more COV peptide antigens disclosed herein, such as, e.g., Polypeptide Structure I, II, or III disclosed herein, SOP¹ and SCP² are each independently a self-cleaving peptide, o is any integer from 0 to 10, and wherein PA¹, PA², and PA³ can each independently encode the same polypeptide or a different polypeptide from each other. In aspects of these embodiments, a broad-spectrum, pan-COV immunogenic composition disclosed herein comprises a nucleotide sequence (e.g., a DNA or RNA) encoding a polypeptide of Polypeptide Structure IV, wherein PA¹ is a first polypeptide disclosed herein, PA² is a second polypeptide disclosed herein, PA³ is a third polypeptide disclosed herein, SOP¹ and SCP² are each independently a self-cleaving peptide, o is any integer from 0 to 5, and wherein PA¹, PA², and PA³ can each independently encode the same polypeptide or a different polypeptide from each other. In other aspects of these embodiments, a broad-spectrum, pan-COV immunogenic composition disclosed herein comprises a nucleotide sequence (e.g., a DNA or RNA) encoding a polypeptide of Polypeptide Structure IV, wherein PA¹ is a first polypeptide disclosed herein, PA² is a second polypeptide disclosed herein, PA³ is a third polypeptide disclosed herein, SOP¹ and SCP² are each independently a self-cleaving peptide, o is any integer from 0 to 3, and wherein PA¹, PA², and PA³ can each independently encode the same polypeptide or a different polypeptide from each other. In yet other aspects of these embodiments, a broad-spectrum, pan-COV immunogenic composition disclosed herein comprises a nucleotide sequence (e.g., a DNA or RNA) encoding a polypeptide of Polypeptide Structure IV, wherein PA¹ is a first polypeptide disclosed herein, PA² is a second polypeptide disclosed herein, PA³ is a third polypeptide disclosed herein, SOP¹ and SCP² are each independently a self-cleaving peptide, o is any integer from 0 to 2, and wherein PA¹, PA², and PA³ can each independently encode the same polypeptide or a different polypeptide from each other. In yet other aspects of these embodiments, a broad-spectrum, pan-COV immunogenic composition disclosed herein comprises a nucleotide sequence (e.g., a DNA or RNA) encoding a polypeptide of Polypeptide Structure IV, wherein PA¹ is a first polypeptide disclosed herein, PA² is a second polypeptide disclosed herein, PA³ is a third polypeptide disclosed herein, SOP¹ and SCP² are each independently a self-cleaving peptide, o is any integer from 0 to 1, and wherein PA¹, PA², and PA³ can each independently encode the same polypeptide or a different polypeptide from each other. In some embodiments, PA¹, PA², and PA³ are each independently Polypeptide Structure I, Polypeptide Structure II, Polypeptide Structure III, or a polypeptide including one or more COV peptide antigens disclosed herein inserted in a LAMP disclosed herein. In the above embodiments, an IRES can be independently operationally linked to the 5′ end of the nucleotide sequence encoding each PA², to the 5′ end of the nucleotide sequence encoding PA³, or to any combination thereof. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce cellular and/or humoral response capable of recognizing infected cells and inducing cytotoxicity and/or antibodies against the one or more Coronavirus species, strains, genotypes, subgenotypes, serotypes or genetic/antigenic lineages.

In aspects of these embodiments, a broad-spectrum, pan-COV immunogenic composition disclosed herein comprises a nucleotide sequence (e.g., a DNA or RNA) encoding a polypeptide of Polypeptide Structure IV, wherein PA¹ is a first polypeptide disclosed herein, PA² is a second polypeptide disclosed herein, PA³ is a third polypeptide disclosed herein, SCP¹ and SCP² are each independently a self-cleaving peptide of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30, o is any integer from 0 to 10, and wherein PA¹, PA², and PA³ can each independently encode the same polypeptide or a different polypeptide from each other. In other aspects of these embodiments, a broad-spectrum, pan-COV immunogenic composition disclosed herein comprises a nucleotide sequence (e.g., a DNA or RNA) encoding a polypeptide of Polypeptide Structure IV, wherein PA¹ is a first polypeptide disclosed herein, PA² is a second polypeptide disclosed herein, PA³ is a third polypeptide disclosed herein, SCP¹ and SCP² are each independently a self-cleaving peptide of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30, o is any integer from 0 to 5, and wherein PA¹, PA², and PA³ can each independently encode the same polypeptide or a different polypeptide from each other. In other aspects of these embodiments, a broad-spectrum, pan-COV immunogenic composition disclosed herein comprises a nucleotide sequence (e.g., a DNA or RNA) encoding a polypeptide of Polypeptide Structure IV, wherein PA¹ is a first polypeptide disclosed herein, PA² is a second polypeptide disclosed herein, PA³ is a third polypeptide disclosed herein, SCP¹ and SCP² are each independently a self-cleaving peptide of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30, o is any integer from 0 to 3, and wherein PA¹, PA², and PA³ can each independently encode the same polypeptide or a different polypeptide from each other. In other aspects of these embodiments, a broad-spectrum, pan-COV immunogenic composition disclosed herein comprises a nucleotide sequence (e.g., a DNA or RNA) encoding a polypeptide of Polypeptide Structure IV, wherein PA¹ is a first polypeptide disclosed herein, PA² is a second polypeptide disclosed herein, PA³ is a third polypeptide disclosed herein, SCP¹ and SCP² are each independently a self-cleaving peptide of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30, o is any integer from 0 to 2, and wherein PA¹, PA², and PA³ can each independently encode the same polypeptide or a different polypeptide from each other. In other aspects of these embodiments, a broad-spectrum, pan-COV immunogenic composition disclosed herein comprises a nucleotide sequence (e.g., a DNA or RNA) encoding a polypeptide of Polypeptide Structure IV, wherein PA¹ is a first polypeptide disclosed herein, PA² is a second polypeptide disclosed herein, PA³ is a third polypeptide disclosed herein, SCP¹ and SCP² are each independently a self-cleaving peptide of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30, o is any integer from 0 to 1, and wherein PA¹, PA², and PA³ can each independently encode the same polypeptide or a different polypeptide from each other. In some embodiments, PA¹, PA², and PA³ are each independently Polypeptide Structure I, Polypeptide Structure II, Polypeptide Structure III, or a polypeptide including one or more COV peptide antigens disclosed herein inserted in a LAMP disclosed herein. In the above embodiments, an IRES can be independently operationally linked to the 5′ end of the nucleotide sequence encoding each PA², to the 5′ end of the nucleotide sequence encoding PA³, or to any combination thereof. In the above embodiments, when administered to an individual, the COV peptide antigen(s) 1) elicit, evoke or otherwise stimulate an immune response against the one or more Coronavirus species, strains, genotypes, subgenotypes, serotypes or genetic/antigenic lineages; or 2) elicit, evoke or otherwise stimulate an immune response against the one or more Coronavirus species, strains, genotypes, subgenotypes, serotypes or genetic/antigenic lineages to produce a cellular and/or humoral response capable of recognizing infected cells and inducing cytotoxicity and/or antibodies against the one or more Coronavirus species, strains, genotypes, subgenotypes, serotypes or genetic/antigenic lineages; or 3) are necessary to elicit, evoke or otherwise stimulate an immune response against one or more Coronavirus species, strains, genotypes, subgenotypes, serotypes or genetic/antigenic lineages; or 4) are necessary to elicit, evoke or otherwise stimulate an immune response against one or more Coronavirus species, strains, genotypes, subgenotypes, serotypes or genetic/antigenic lineages to produce a cellular and/or humoral response capable of recognizing infected cells and inducing cytotoxicity and/or antibodies against the one or more Coronavirus species, strains, genotypes, subgenotypes, serotypes or genetic/antigenic lineages.

In some embodiments, a broad-spectrum, pan-COV immunogenic composition disclosed herein comprises a nucleotide sequence (e.g., a DNA or RNA) encoding a polypeptide of Polypeptide Structure IV, wherein PA¹ is SEQ ID NO: 31, PA² is SEQ ID NO: 31, PA³ is SEQ ID NO: 31, SCP¹ and SCP² are each independently a self-cleaving peptide, and o is 1. In some embodiments, a broad-spectrum, pan-COV immunogenic composition disclosed herein comprises a nucleotide sequence (e.g., a DNA or RNA) encoding a polypeptide of Polypeptide Structure IV, wherein PA¹ is SEQ ID NO: 31, PA² is SEQ ID NO: 31, PA³ is SEQ ID NO: 31, SCP¹ and SCP² are each independently a self-cleaving peptide of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30, and o is 1. In some embodiments, a broad-spectrum, pan-COV immunogenic composition disclosed herein comprises a nucleotide sequence (e.g., a DNA or RNA) encoding a polypeptide of Polypeptide Structure IV, wherein PA¹ is SEQ ID NO: 31, PA² is SEQ ID NO: 31, PA³ is SEQ ID NO: 31, SCP¹ and SCP² are each independently a self-cleaving peptide of SEQ ID NO: 27, and o is 1. In the above embodiments, an IRES can be independently operationally linked to the 5′ end of the nucleotide sequence encoding each PA², to the 5′ end of the nucleotide sequence encoding PA³, or to any combination thereof.

In some embodiments, a broad-spectrum, pan-COV immunogenic composition disclosed herein comprises a nucleotide sequence (e.g., a DNA or RNA) encoding a polypeptide of Polypeptide Structure IV, wherein PA¹ is SEQ ID NO: 31, PA³ is SEQ ID NO: 31, SCP¹ is a self-cleaving peptide, and o is 0. In some embodiments, a broad-spectrum, pan-COV immunogenic composition disclosed herein comprises a nucleotide sequence (e.g., a DNA or RNA) encoding a polypeptide of Polypeptide Structure IV, wherein PA¹ is SEQ ID NO: 31, PA³ is SEQ ID NO: 31, SCP¹ is a self-cleaving peptide of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30, and o is 0. In some embodiments, a broad-spectrum, pan-COV immunogenic composition disclosed herein comprises a nucleotide sequence (e.g., a DNA or RNA) encoding a polypeptide of Polypeptide Structure IV, wherein PA¹ is SEQ ID NO: 31, PA³ is SEQ ID NO: 31, SCP¹ is a self-cleaving peptide of SEQ ID NO: 27, and o is 0. In the above embodiments, an IRES can be independently operationally linked to the 5′ end of the nucleotide sequence encoding PA³.

In some embodiments, a broad-spectrum, pan-COV immunogenic composition disclosed herein comprises a nucleotide sequence (e.g., a DNA or RNA) encoding a polypeptide of Polypeptide Structure IV, wherein PA¹ is SEQ ID NO: 31, PA³ is SEQ ID NO: 33, SCP¹ is a self-cleaving peptide, and o is 0. In some embodiments, a broad-spectrum, pan-COV immunogenic composition disclosed herein comprises a nucleotide sequence (e.g., a DNA or RNA) encoding a polypeptide of Polypeptide Structure IV, wherein PA¹ is SEQ ID NO: 31, PA³ is SEQ ID NO: 33, SCP¹ is a self-cleaving peptide of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30, and o is 0. In some embodiments, a broad-spectrum, pan-COV immunogenic composition disclosed herein comprises a nucleotide sequence (e.g., a DNA or RNA) encoding a polypeptide of Polypeptide Structure IV, wherein PA¹ is SEQ ID NO: 31, PA³ is SEQ ID NO: 33, SCP¹ is a self-cleaving peptide of SEQ ID NO: 27, and o is 0. In the above embodiments, an IRES can be independently operationally linked to the 5′ end of the nucleotide sequence encoding PA³.

In some embodiments, a nucleotide sequence disclosed herein is itself an active ingredient of a broad-spectrum, pan-COV immunogenic composition disclosed herein. In these embodiments, expression of the encoded polypeptide, such as, e.g., one or more of Polypeptide Structures I-IV utilizes the in vivo mechanisms contained in the cells of an individual being administered the broad-spectrum, pan-COV immunogenic composition. For example, a DNA nucleotide sequence would use the transcriptional and translation machinery of the cells while a RNA nucleotide sequence would use the translational machinery of the cells.

In some embodiments, a nucleotide sequence disclosed herein is cloned into an expression construct and the resulting recombinant protein produced would be an active ingredient of a broad-spectrum, pan-COV immunogenic composition disclosed herein. In these embodiments, a polypepeptide, such as, e.g., one or more of Polypeptide Structures I-IV encoded by a nucleotide sequence disclosed herein would be expressed using protein expression protocols based on standard molecular genetic techniques.

In some embodiments, an expression construct is produced using a nucleotide sequence disclosed herein operably-linked to an expression vector useful for expressing the nucleotide sequence in a cell or cell-free extract. A wide variety of expression vectors can be employed for expressing a nucleotide sequence encoding a polypeptide, such as, e.g., one or more of Polypeptide Structures I-IV, including, without limitation, a viral expression vector; a prokaryotic expression vector; eukaryotic expression vectors, such as, e.g., a yeast expression vector, an insect expression vector and a mammalian expression vector; and a cell-free extract expression vector. It is further understood that useful expression vectors may include controlling elements, such as, e.g., a constitutive, tissue-specific, cell-specific or inducible promoter element, enhancer element or both. Non-limiting examples of expression vectors, along with well-established reagents and conditions for making and using an expression construct from such expression vectors are readily available from commercial vendors that include, without limitation, BD Biosciences-Clontech, Palo Alto, Calif.; BD Biosciences Pharmingen, San Diego, Calif.; Invitrogen, Inc, Carlsbad, Calif.; EMD Biosciences-Novagen, Madison, Wis.; QIAGEN, Inc., Valencia, Calif.; and Stratagene, La Jolla, Calif. Expression vectors and methods and uses of protein expression systems are described in, e.g., U.S. Pat. Nos. 5,912,141, 6,025,158, 6,071,722, 6,221,644, 6,287,801, 6,448,044, 6,514,729, 6,635,459, 6,759,211, 6,770,459, 7,527,801, 7,655,247, 7,740,868, 7,811,584, 7,892,565, 7,964,200, 7,993,656, 8,067,200, 8,187,834, 8,685,411, 9,044,420, 9,371,366, 9,428,739, 9,902,761, and 10,420,829, the content of each of which is hereby incorporated by reference in its entirety.

A broad-spectrum, pan-COV immunogenic composition disclosed herein may optionally and further comprise one or more adjuvants. As such, a broad-spectrum, pan-COV immunogenic composition disclosed herein may be formulated or administered alone or in conjunction with one or more other adjuvant. An adjuvant is any substance or mixture of substances that increases or diversifies the immune response to a COV peptide antigen disclosed herein. An adjuvant may serve to reduce the number of immunizations or the amount of antigen required for protective immunization. Non-limiting adjuvants include organic adjuvants, inorganic adjuvants, or combinations thereof. An adjuvant disclosed herein can also be part of a vaccine adjuvant-delivery system (VADS), with non-limiting examples being liposomes (LPs) and nanoparticles (NPs). An adjuvant disclosed herein can be provided as a separate expression construct, the separate expression construct encoding the adjuvant. Adjuvants and methods of making and using such adjuvants are described in, e.g., Gupta et al, Vaccine 11: 993-306 (1993); Powell, M. F. and Newman, M. J. (Eds.), Vaccine Design, The Subunit and Adjuvant Approach, pp. 949 (Pharmaceutical Biotechnology, Vol. 6, Plenum Press, 1995); and Arnon, R. (Ed.), Synthetic Vaccines, pp. 352 (CRC Press, Inc., 1996); O'Hagan (Ed.), Vaccine Adjuvants: Preparation Methods and Research Protocols, pp. 356 (Humana Press, 2000); Hackett, C. J. and Haran, D. A. (Eds.), Vaccine Adjuvants: Immunological and Clinical Principles, pp. 296 (Humana Press, 2007); Reed, S. G. (Ed.), Emulsion-based Vaccine Adjuvants, pp. 54 (Future Medicine Ltd., 2012); and Wang, N. and Wang, T. (Eds.), Immunization: Vaccine Adjuvant Delivery System and Strategies, pp. 122 (IntechOpen, 2018), the content of each of which is hereby incorporated by reference in its entirety.

In some embodiments, a broad-spectrum, pan-COV immunogenic composition disclosed herein is with an organic adjuvant. In some embodiments, a broad-spectrum, pan-COV immunogenic composition disclosed herein is with an inorganic adjuvant. In some embodiments, a broad-spectrum, pan-COV immunogenic composition disclosed herein is with both an organic and inorganic adjuvant.

Organic adjuvants include, without limitation, emulsion adjuvants, pathogen associated molecular patterns (PAMPs), molecular adjuvants, poly (acrylic acid) adjuvants like carbopol; polyphosphazenes (PZs) adjuvants like poly[di(carboxylatophenoxy)phosphazene] (PCPP) and poly[di(sodiumcarboxylatoethylphenoxy)phosphazene] (PCEP). In some embodiments, a broad-spectrum, pan-COV immunogenic composition disclosed herein comprises one or more adjuvants. In some embodiments, a broad-spectrum, pan-COV immunogenic composition disclosed herein do not include one or more adjuvants; an adjuvant free immunogenic composition. General considerations in the formulation and/or manufacture of pharmaceutical agents, broad-spectrum, pan-COV immunogenic compositions disclosed herein, may be found, e.g., in Remington: The Science and Practice of Pharmacy 21st ed., Lippincott Williams & Wilkins, 2005, the content of which is hereby incorporated by reference in its entirety. In some embodiments, a broad-spectrum, pan-COV immunogenic composition disclosed herein is with a poly (acrylic acid) adjuvant. In some embodiments, a broad-spectrum, pan-COV immunogenic composition disclosed herein is with a polyphosphazene adjuvant.

In some embodiments, a broad-spectrum, pan-COV immunogenic composition disclosed herein is with an emulsion adjuvant. An emulsion adjuvant is a mixture of two or more immiscible fluids, with tiny particles or “droplets” of one liquid suspended in another. Chemically, they are colloids where both phases are fluids. In an emulsion, one liquid (the dispersed phase) is dispersed in the other (the continuous phase). The boundary between these phases is called the interface. Non-limiting examples of emulsion adjuvants include oil-in-water (o/w) emulsion adjuvants like Freund type of adjuvants, such as, e.g., Freund's complete adjuvant (FCA); Freund's incomplete adjuvant (FIA); water-in-oil (w/o) emulsion adjuvants, such as, e.g., water-in-oil (w/o) emulsions containing oil and surfactants like a Montanide (mineral oil and surfactant from the mannide monooleate) or emulsions containing squalene oil and surfactant like MF59; sapogenin glycosides, such as, e.g., saponins. In some embodiments, a broad-spectrum, pan-COV immunogenic composition disclosed herein may be formulated in a water-in-oil emulsion adjuvant comprising a continuous hydrophobic phase in which the hydrophilic phase is dispersed.

In some embodiments, a broad-spectrum, pan-COV immunogenic composition disclosed herein is with a PAMP adjuvant. PAMPs small molecular motifs recognized by toll-like receptors (TLRs) and other pattern recognition receptors (PRRs) in both plants and animals. PAMPs activate innate immune responses, protecting the host from infection, by identifying some conserved non-self molecules. Non-limiting examples of PAMPs include lipopolysaccharides (LPSs, also known as endotoxins); LPS extracts like monophosphoryl lipid A (MPL or MPLA), glucopyranosyl lipid adjuvant (GLA), or aminoalkyl glucopyranosides; flagellin polypeptides and hinge regions (also referred to as “D3 domain or region, “propeller domain or region,” “hypervariable domain or region” and “variable domain or region.”) thereof, like flagellin polypeptides and hinge regions thereof from S. Typhimurium, H. Pylori, V. Cholera, S. marcesens, S. flexneri, T. Pallidum, L. pneumophila, B. burgdorferei, C. difficile, R. meliloti, A. tumefaciens, R. lupini, B. clarridgeiae, P. Mirabilis, B. subtilus, L. monocytogenes, P. aeruginosa, and E. coli; nucleotide-based adjuvants like ssRNA agonists like TLR7 and TLR8 and oligonucleotide motifs like poly Inosine:Cytosine (poly I:C) and cytosine phosphoguanine (CpG); and N-acetylmuramyl-L-alanyl-D-isoglutamine (commonly known as muramyl dipeptide or “MDP”). PAMPs and methods and uses of PAMPs are described in, e.g., U.S. Pat. Nos. 5,462,735, 5,723,130, 5,705,161, 5,750,110, 5,776,468, 5,961,985, 5,997,881, 6,027,730, 6,130,082, 6,146,632, 6,267,968, 6,306,404, 6,803,042, 7,147,862, 7,408,050, 7,745,598, 8,110,203, 8,546,550, 8,968,749, 9,241,954, 9,296,792, 9,603,919, 9,796,762, 9,950,025, 9,956,284, 10,202,426, 10,245,318, 10,279,025, 10,336,793, 10,576,147, 10,669,316, 10,730,915, 10,736,913, the content of each of which is hereby incorporated by reference in its entirety. In some embodiments, a PAMP disclosed herein is combined with one or more COV antigens disclosed herein as an admixture like a COV antigen-MLP admixture. In some embodiments, a PAMP disclosed herein is combined with one or more COV antigens disclosed herein as a fusion protein like a COV antigen-flagellin polypeptide fusion protein.

In some embodiments, a broad-spectrum, pan-COV immunogenic composition disclosed herein is with a molecular adjuvant. Molecular adjuvants are adjuvating immunomodulatory proteins. Molecular adjuvants can be provided as separate expression constructs and comprise signaling molecules including cytokines, chemokines, immune costimulatory molecules, toll-like receptor agonists, or inhibitors of immune suppressive pathways. Non-limiting examples of molecular adjuvants include cytokines like CCL21, GM-CSF, IFN-γ, IL-2, IL-4, IL-7, IL-10, IL-12, IL-15, IL-18, and M-CSF, chemokines like IL-8, MCP-1, MIP-1α, and RANTES, or immune costimulatory molecules like CD40L, CD80/86, and ICAM-1. Molecular adjuvants and methods and uses of molecular adjuvants are described in, e.g., U.S. Pat. Nos. 4,963,354, 5,891,432, 5,980,911, 6,096,320, 6,569,418, 6,811,780, 6,919,319, 6,977,072, 7,132,407, 7,347,996, 7,384,641, 7,897,152, 8,007,776, 8,053,231, 8,716,254, 8,802,076, 8,932,575, the content of each of which is hereby incorporated by reference in its entirety.

Inorganic adjuvants include, without limitation, inorganic salts, such as, e.g., aluminum-based adjuvants (or alum-based adjuvants) like aluminum hydroxide (Al(OH)₃), aluminum hydroxyphosphate sulfate (AAHS), aluminum phosphate, potassium aluminium sulfate (KAI(SO₄), or Alum); calcium salts like calcium phosphate or calcium chloride; colloidal iron hydroxide; zirconium salts, and zinc sulfate. In some embodiments, a broad-spectrum, pan-COV immunogenic composition disclosed herein is with an aluminum-based adjuvant.

In some embodiments, a broad-spectrum, pan-COV immunogenic composition disclosed herein comprises a single adjuvant disclosed herein. In one embodiment, a broad-spectrum, pan-COV immunogenic composition disclosed herein comprises a plurality of adjuvants disclosed herein. In aspects of this embodiment, a broad-spectrum, pan-COV immunogenic composition disclosed herein comprises, e.g., 1, 2, 3, 4, or 5 adjuvants disclosed herein. In other aspects of this embodiment, an immunogenic composition disclosed herein comprises, e.g., at least 1, at least 2, at least 3, at least 4, or at least 5 adjuvants disclosed herein. In yet other aspects of this embodiment, a broad-spectrum, pan-COV immunogenic composition disclosed herein comprises, e.g., at most 1, at most 2, at most 3, at most 4, or at most 5 adjuvants disclosed herein. In still other aspects of this embodiment, a broad-spectrum, pan-COV immunogenic composition disclosed herein comprises, e.g., 1 to 2, 1 to 3, 1 to 4, 1 to 5, 2 to 3, 2 to 4, 2 to 5, 3 to 4, 3 to 5, or 4 to 5 adjuvants disclosed herein.

In some embodiments, an adjuvant disclosed herein typically comprises half the volume of the administered dose. Typically, a broad-spectrum, pan-COV immunogenic composition disclosed herein comprising an adjuvant will be administered in a dose of about 0.25 mL to about 1 mL. Thus, if a broad-spectrum, pan-COV immunogenic composition disclosed herein is administered in about 1 mL dose, about 0.5 mL of this composition will be an adjuvant. Similarly, if a broad-spectrum, pan-COV immunogenic composition disclosed herein is administered in about 0.5 mL dose, about 0.25 mL of this composition will be an adjuvant.

The amount of an adjuvant disclosed herein included in a broad-spectrum, pan-COV immunogenic composition disclosed herein is an amount effective in increasing an appropriate immune response of the targeted Coronavirus species, strains, genotypes, subgenotypes, serotypes or genetic/antigenic lineages in the individual. Typically, this amount is also one that does not cause significant adverse side effects. Such amount will vary depending on which adjuvant or adjuvants are employed. An optimal amount of an adjuvant for a particular broad-spectrum, pan-COV immunogenic composition disclosed herein can be ascertained by standard studies involving observation of antibody titers and other responses in individuals.

Generally, an effective and safe dose of adjuvant disclosed herein varies from about 100 μg/mL to about 1,500 μg/mL concentration. In aspects of this embodiment, a broad-spectrum, pan-COV immunogenic composition disclosed herein comprises an adjuvant disclosed herein in an amount of, e.g., about 100 μg/mL, about 200 μg/mL, about 300 μg/mL, about 400 μg/mL, about 500 μg/mL, about 600 μg/mL, about 700 μg/mL, about 800 μg/mL, about 900 μg/mL, about 1,000 μg/mL, about 1,100 μg/mL, about 1,200 μg/mL, about 1,300 μg/mL, about 1,400 μg/mL, or about 1,500 μg/mL. In other aspects of this embodiment, a broad-spectrum, pan-COV immunogenic composition disclosed herein comprises an adjuvant disclosed herein in an amount of, e.g., at least 100 μg/mL, at least 200 μg/mL, at least 300 μg/mL, at least 400 μg/mL, at least 500 μg/mL, at least 600 μg/mL, at least 700 μg/mL, at least 800 μg/mL, at least 900 μg/mL, at least 1,000 μg/mL, at least 1,100 μg/mL, at least 1,200 μg/mL, at least 1,300 μg/mL, at least 1,400 μg/mL, or at least 1,500 μg/mL. In yet other aspects of this embodiment, a broad-spectrum, pan-COV immunogenic composition disclosed herein comprises an adjuvant disclosed herein in an amount of, e.g., at most 100 μg/mL, at most 200 μg/mL, at most 300 μg/mL, at most 400 μg/mL, at most 500 μg/mL, at most 600 μg/mL, at most 700 μg/mL, at most 800 μg/mL, at most 900 μg/mL, at most 1,000 μg/mL, at most 1,100 μg/mL, at most 1,200 μg/mL, at most 1,300 μg/mL, at most 1,400 μg/mL, or at most 1,500 μg/mL.

In yet other aspects of this embodiment, a broad-spectrum, pan-COV immunogenic composition disclosed herein comprises an adjuvant disclosed herein in an amount of, e.g., about 100 μg/mL to about 200 μg/mL, about 100 μg/mL to about 300 μg/mL, about 100 μg/mL to about 400 μg/mL, about 100 μg/mL to about 500 μg/mL, about 100 μg/mL to about 600 μg/mL, about 100 μg/mL to about 700 μg/mL, about 100 μg/mL to about 800 μg/mL, about 100 μg/mL to about 900 μg/mL, about 100 μg/mL to about 1,000 μg/mL, about 100 μg/mL to about 1,200 μg/mL, about 100 μg/mL to about 1,300 μg/mL, about 100 μg/mL to about 1,400 μg/mL, about 100 μg/mL to about 1,500 μg/mL, about 200 μg/mL to about 300 μg/mL, about 200 μg/mL to about 400 μg/mL, about 200 μg/mL to about 500 μg/mL, about 200 μg/mL to about 600 μg/mL, about 200 μg/mL to about 700 μg/mL, about 200 μg/mL to about 800 μg/mL, about 200 μg/mL to about 900 μg/mL, about 200 μg/mL to about 1,000 μg/mL, about 200 μg/mL to about 1,200 μg/mL, about 200 μg/mL to about 1,300 μg/mL, about 200 μg/mL to about 1,400 μg/mL, about 200 μg/mL to about 1,500 μg/mL, about 300 μg/mL to about 400 μg/mL, about 300 μg/mL to about 500 μg/mL, about 300 μg/mL to about 600 μg/mL, about 300 μg/mL to about 700 μg/mL, about 300 μg/mL to about 800 μg/mL, about 300 μg/mL to about 900 μg/mL, about 300 μg/mL to about 1,000 μg/mL, about 300 μg/mL to about 1,200 μg/mL, about 300 μg/mL to about 1,300 μg/mL, about 300 μg/mL to about 1,400 μg/mL, about 300 μg/mL to about 1,500 μg/mL, about 400 μg/mL to about 500 μg/mL, about 400 μg/mL to about 600 μg/mL, about 400 μg/mL to about 700 μg/mL, about 400 μg/mL to about 800 μg/mL, about 400 μg/mL to about 900 μg/mL, about 400 μg/mL to about 1,000 μg/mL, about 400 μg/mL to about 1,200 μg/mL, about 400 μg/mL to about 1,300 μg/mL, about 400 μg/mL to about 1,400 μg/mL, about 400 μg/mL to about 1,500 μg/mL, about 500 μg/mL to about 600 μg/mL, about 500 μg/mL to about 700 μg/mL, about 500 μg/mL to about 800 μg/mL, about 500 μg/mL to about 900 μg/mL, about 500 μg/mL to about 1,000 μg/mL, about 500 μg/mL to about 1,200 μg/mL, about 500 μg/mL to about 1,300 μg/mL, about 500 μg/mL to about 1,400 μg/mL, about 500 μg/mL to about 1,500 μg/mL, about 600 μg/mL to about 700 μg/mL, about 600 μg/mL to about 800 μg/mL, about 600 μg/mL to about 900 μg/mL, about 600 μg/mL to about 1,000 μg/mL, about 600 μg/mL to about 1,200 μg/mL, about 600 μg/mL to about 1,300 μg/mL, about 600 μg/mL to about 1,400 μg/mL, about 600 μg/mL to about 1,500 μg/mL, about 700 μg/mL to about 800 μg/mL, about 700 μg/mL to about 900 μg/mL, about 700 μg/mL to about 1,000 μg/mL, about 700 μg/mL to about 1,200 μg/mL, about 700 μg/mL to about 1,300 μg/mL, about 700 μg/mL to about 1,400 μg/mL, about 700 μg/mL to about 1,500 μg/mL, about 800 μg/mL to about 900 μg/mL, about 800 μg/mL to about 1,000 μg/mL, about 800 μg/mL to about 1,200 μg/mL, about 800 μg/mL to about 1,300 μg/mL, about 800 μg/mL to about 1,400 μg/mL, about 800 μg/mL to about 1,500 μg/mL, about 900 μg/mL to about 1,000 μg/mL, about 900 μg/mL to about 1,200 μg/mL, about 900 μg/mL to about 1,300 μg/mL, about 900 μg/mL to about 1,400 μg/mL, about 900 μg/mL to about 1,500 μg/mL, about 1,000 μg/mL to about 1,200 μg/mL, about 1,000 μg/mL to about 1,300 μg/mL, about 1,000 μg/mL to about 1,400 μg/mL, about 1,000 μg/mL to about 1,500 μg/mL, about 1,100 μg/mL to about 1,200 μg/mL, about 1,100 μg/mL to about 1,300 μg/mL, about 1,100 μg/mL to about 1,400 μg/mL, about 1,100 μg/mL to about 1,500 μg/mL, about 1,200 μg/mL to about 1,300 μg/mL, about 1,200 μg/mL to about 1,400 μg/mL, about 1,200 μg/mL to about 1,500 μg/mL, about 1,300 μg/mL to about 1,400 μg/mL, about 1,300 μg/mL to about 1,500 μg/mL, or about 1,400 μg/mL to about 1,500 μg/mL.

In aspects of this embodiment, a broad-spectrum, pan-COV immunogenic composition disclosed herein comprises an adjuvant disclosed herein in an antigen:adjuvant ratio of, e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9 or 1:10. In other aspects of this embodiment, a broad-spectrum, pan-COV immunogenic composition disclosed herein comprises an adjuvant disclosed herein in an antigen:adjuvant ratio range of, e.g., 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 1:1 to 1:10, 1:2 to 1:10, 1:3 to 1:10, 1:4 to 1:10, 1:5 to 1:10, 1:6 to 1:10, 1:7 to 1:10, 1:8 to 1:10, 1:9 to 1:10, 10:1 to 1:9, 9:1 to 1:9, 8:1 to 1:9, 7:1 to 1:9, 6:1 to 1:9, 5:1 to 1:9, 4:1 to 1:9, 3:1 to 1:9, 2:1 to 1:9, 1:1 to 1:9, 1:2 to 1:9, 1:3 to 1:9, 1:4 to 1:9, 1:5 to 1:9, 1:6 to 1:9, 1:7 to 1:9, 1:8 to 1:9, 10:1 to 1:8, 9:1 to 1:8, 8:1 to 1:8, 7:1 to 1:8, 6:1 to 1:8, 5:1 to 1:8, 4:1 to 1:8, 3:1 to 1:8, 2:1 to 1:8, 1:1 to 1:8, 1:2 to 1:8, 1:3 to 1:8, 1:4 to 1:8, 1:5 to 1:8, 1:6 to 1:8, 1:7 to 1:8, 10:1 to 1:7, 9:1 to 1:7, 8:1 to 1:7, 7:1 to 1:7, 6:1 to 1:7, 5:1 to 1:7, 4:1 to 1:7, 3:1 to 1:7, 2:1 to 1:7, 1:1 to 1:7, 1:2 to 1:7, 1:3 to 1:7, 1:4 to 1:7, 1:5 to 1:7, 1:6 to 1:7, 10:1 to 1:6, 9:1 to 1:6, 8:1 to 1:6, 7:1 to 1:6, 6:1 to 1:6, 5:1 to 1:6, 4:1 to 1:6, 3:1 to 1:6, 2:1 to 1:6, 1:1 to 1:6, 1:2 to 1:6, 1:3 to 1:6, 1:4 to 1:6, 1:5 to 1:6, 10:1 to 1:5, 9:1 to 1:5, 8:1 to 1:5, 7:1 to 1:5, 6:1 to 1:5, 5:1 to 1:5, 4:1 to 1:5, 3:1 to 1:5, 2:1 to 1:5, 1:1 to 1:5, 1:2 to 1:5, 1:3 to 1:5, 1:4 to 1:5, 10:1 to 1:4, 9:1 to 1:4, 8:1 to 1:4, 7:1 to 1:4, 6:1 to 1:4, 5:1 to 1:4, 4:1 to 1:4, 3:1 to 1:4, 2:1 to 1:4, 1:1 to 1:4, 1:2 to 1:4, 1:3 to 1:4, 10:1 to 1:3, 9:1 to 1:3, 8:1 to 1:3, 7:1 to 1:3, 6:1 to 1:3, 5:1 to 1:3, 4:1 to 1:3, 3:1 to 1:3, 2:1 to 1:3, 1:1 to 1:3, 1:2 to 1:3, 10:1 to 1:2, 9:1 to 1:2, 8:1 to 1:2, 7:1 to 1:2, 6:1 to 1:2, 5:1 to 1:2, 4:1 to 1:2, 3:1 to 1:2, 2:1 to 1:2, 1:1 to 1:2, 10:1 to 1:1, 9:1 to 1:1, 8:1 to 1:1, 7:1 to 1:1, 6:1 to 1:1, 5:1 to 1:1, 4:1 to 1:1, 3:1 to 1:1, 2:1 to 1:1, 10:1 to 2:1, 9:1 to 2:1, 8:1 to 2:1, 7:1 to 2:1, 6:1 to 2:1, 5:1 to 2:1, 4:1 to 2:1, 3:1 to 2:1, 10:1 to 3:1, 9:1 to 3:1, 8:1 to 3:1, 7:1 to 3:1, 6:1 to 3:1, 5:1 to 3:1, 4:1 to 3:1, 10:1 to 4:1, 9:1 to 4:1, 8:1 to 4:1, 7:1 to 4:1, 6:1 to 4:1, 5:1 to 4:1, 10:1 to 5:1, 9:1 to 5:1, 8:1 to 5:1, 7:1 to 5:1, 6:1 to 5:1, 10:1 to 6:1, 9:1 to 6:1, 8:1 to 6:1, 7:1 to 6:1, 10:1 to 7:1, 9:1 to 7:1, 8:1 to 7:1, 10:1 to 8:1, 9:1 to 8:1, or 10:1 to 9:1 L.

A broad-spectrum, pan-COV immunogenic composition disclosed herein can be formulated using one or more carriers in any manner that enables sufficient delivery of one or more COV antigens disclosed herein to enable immunization or vaccination of an individual against a COV infection. A carrier denotes an organic or inorganic compound, natural or synthetic, with which an active ingredient (such as, e.g., one or more COV peptide antigens disclosed herein and/or one or more nucleotide sequences encoding one or more COV peptide antigens disclosed herein) is combined to facilitate administration of a broad-spectrum, pan-COV immunogenic composition disclosed herein. Such formulations of an active ingredient and carrier can be designated as a vaccine delivery system (VDS). A VDS disclosed herein whose carrier or components thereof not only facilitates administration of a broad-spectrum, pan-COV immunogenic composition disclosed herein but also function as an adjuvant is designated a vaccine adjuvant-delivery system (VADS). A VDS or VADS can be formulated for an oral route of administration, such as, e.g., enteral, buccal, respiratory or intranasal, a dermal route of administration, such as, e.g., topical or transdermal, or a parenteral route of administration, such as, e.g., injection like intramuscular, subcutaneous, and intradermal injection or infusion like intramuscular, subcutaneous, intradermal and intravenous infusion.

In some embodiments, a broad-spectrum, pan-COV immunogenic composition disclosed herein is formulated as a VDS, which can optionally comprise an adjuvant disclosed herein. In aspects of these embodiments, a broad-spectrum, pan-COV immunogenic composition disclosed herein is formulated as a VDS, which can optionally comprise an adjuvant disclosed herein. In aspects of these embodiments, a broad-spectrum, pan-COV immunogenic composition disclosed herein is formulated as a VDS, which can optionally comprise an adjuvant disclosed herein.

In some embodiments, a broad-spectrum, pan-COV immunogenic composition disclosed herein is formulated as a VADS. In aspects of these embodiments, a broad-spectrum, pan-COV immunogenic composition disclosed herein is formulated as a VADS. In aspects of these embodiments, a broad-spectrum, pan-COV immunogenic composition disclosed herein is formulated as a VADS.

A carrier disclosed herein is generally a colloidal carrier, such as, e.g., emulsion carriers, liposome carriers, niosome carriers, microsphere carriers, nanoparticle carriers, carbon nanotube (CNTs, or carbon nanofibers) carriers, and peptide carriers. Emulsion carriers can function by encapsulating one or more COV antigens disclosed herein or be admixed with one or more COV antigens disclosed herein. Emulsion carriers and methods and uses of emulsion carriers are described in, e.g., U.S. Pat. Nos. 9,295,646, 9,636,410, 10,307,374, the content of each of which is hereby incorporated by reference in its entirety.

In some embodiments, a broad-spectrum, pan-COV immunogenic composition disclosed herein is formulated with a liposome carrier. In aspects of these embodiments, a broad-spectrum, pan-COV immunogenic composition disclosed herein is formulated with a liposome carrier. In aspects of these embodiments, a broad-spectrum, pan-COV immunogenic composition disclosed herein is formulated with a liposome carrier. Liposome carriers comprise amphiphilic lipids that form a lipid bilayer in an aqueous environment that encapsulates one or more COV antigens disclosed herein within an aqueous core or can be admixed with one or more COV antigens disclosed herein. These amphiphilic lipids can have an anionic, cationic or zwitterionic hydrophilic head group. The lipids can be saturated or unsaturated. Liposomes can be formed from a single lipid or from a mixture of lipids. A mixture of lipids may comprise (i) a mixture of anionic lipids (ii) a mixture of cationic lipids (iii) a mixture of zwitterionic lipids (iv) a mixture of anionic lipids and cationic lipids (v) a mixture of anionic lipids and zwitterionic lipids (vi) a mixture of zwitterionic lipids and cationic lipids or (vii) a mixture of anionic lipids, cationic lipids and zwitterionic lipids. Liposome carriers include small unilamellar vesicles (SUVs), large unilamellar vesicles (LUVs), multilamellar vesicles (MLVs), and multivesicular liposomes (MVLs). Liposome carriers include, without limitation, cholesterol (CHO), phospholipids such as phosphatidylcholine (PC), phosphatidylserine (PS), phosphatidylglycerol (PG), sphingomyelin (SM), 1,2-Dimyristoyl-sn-glycero-3-phosphate (DMPG), 1,2-Dioleoyl-sn-glycero-3-phosphocholine (DOPC), Dioleoyl-phosphatidylethanolamine (DOPE), 1,2-Dipalmitoyl-sn-glycero-3-phosphocholine (DPPC), 1,2-Distearoyl-sn-glycero-3-phosphocholine (DSPC), 2-distearoyl-sn-glycero-3-phosphoethanolamine (DSPE), 1-palmitoyl-2-oleoyl-sn-glycero-3-phosphocholine (POPC), and 1-palmitoyl-2-oleoyl-sn-glycero-3-phosphoethanolamine (POPE); and other charged lipids such as 3 (N,N-dimethylaminoethane)-carbamyl cholesterol (DMACHO), N[1-(2,3-dioleyloxy) propyl]-N,N,N-triethylammonium (DOTMA), 1,2-Dioleoyl-3-trimethylammoniumpropane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLin-DMA), 1,2-dilinolenyloxy-N,N-dimethyl-3-aminopropane (DLen-DMA), and stearylamine (SA). Cationic lipids include, without limitation, DOTAP, DSDMA, DODMA, DLin-DMA, DLen-DMA. Zwitterionic lipids include, but are not limited to, acyl zwitterionic lipids and ether zwitterionic lipids. Examples of useful zwitterionic lipids are DPPC, DOPC and dodecylphosphocholine. Liposome carriers and methods and uses of liposome carriers are described in, e.g., U.S. Pat. Nos. 4,235,871, 4,241,046, 5,277,914, 5,549,910, 5,567,434, 8,765,171, 9,254,265, 9,364,563, 9,801,987, 9,931,419, 10,532,067, the content of each of which is hereby incorporated by reference in its entirety. Liposome carriers also include lipid-like materials termed lipidoid carriers as described in, e.g., U.S. Pat. Nos. 8,450,298, 8,969,353, 9,556,110, 9,840,479, and 10,189,802, the content of each of which is hereby incorporated by reference in its entirety; and lipoplex carriers as described in, e.g., U.S. Pat. Nos. 7,811,603, 9,855,345, the content of each of which is hereby incorporated by reference in its entirety.

In some embodiments, a broad-spectrum, pan-COV immunogenic composition disclosed herein is formulated with a niosome carrier. In aspects of these embodiments, a broad-spectrum, pan-COV immunogenic composition disclosed herein is formulated with a niosome carrier. In aspects of these embodiments, a broad-spectrum, pan-COV immunogenic composition disclosed herein is formulated with a niosome carrier. Niosome carriers can function by encapsulating one or more COV antigens disclosed herein or be admixed with one or more COV antigens disclosed herein. Niosome carriers comprise surfactants and lipids and like liposomes, form a lipid bilayer. In some embodiments, niosome carriers comprise non-ionic surfactants and cholesterol. Non-limiting examples of non-ionic surfactants useful in preparing noisome carriers include surfactants of the alkyl or dialkyl polyglycerol ether class like polysorbates. In some embodiments, niosome carriers comprise cationic surfactants and an apolar fraction or part of said apolar fraction of a total lipid extract of a mycobacterium. Non-limiting examples of cationic surfactants useful in preparing noisome carriers include dimethyldioctadecylammonium-bromide, dimethyldioctadecylammonium-chloride, di methyldioctadecylammonium-phosphate and dimethyldioctadecylammonium-acetate (DDA). Niosome carriers and methods and uses of niosome carriers are described in, e.g., U.S. Pat. Nos. 4,830,857, 7,981,442, 8,241,610, 8,435,558, the content of each of which is hereby incorporated by reference in its entirety.

In some embodiments, a broad-spectrum, pan-COV immunogenic composition disclosed herein is formulated with a nanoparticle carrier. In aspects of these embodiments, a broad-spectrum, pan-COV immunogenic composition disclosed herein is formulated with a nanoparticle carrier. In aspects of these embodiments, a broad-spectrum, pan-COV immunogenic composition disclosed herein is formulated with a nanoparticle carrier. Nanoparticle carriers can function by encapsulating one or more COV antigens disclosed herein, conjugating one or more COV antigens disclosed herein to their surface via a linker, adsorbing one or more COV antigens disclosed herein to their surface, or be admixed with one or more COV antigens disclosed herein. Nanoparticle carriers can be core-shell nanoparticle carriers. Nanoparticle carriers can be formulated for controlled and/or sustained release of one or more nucleotide sequences disclosed herein as described in, e.g., U.S. Pat. Nos. 5,656,298, 5,861,159, 7,550,441, 7,858,093, 9,539,212, 10,034,835, and US Publication No. 2010/0303850, the content of each of which is hereby incorporated by reference in its entirety. In some embodiments, a broad-spectrum, pan-COV immunogenic composition disclosed herein may be formulated for controlled and/or sustained release wherein the formulation comprises at least one polymer that is a crystalline side chain (CYSC) polymer as described in U.S. Pat. No. 8,399,007, the content of which is hereby incorporated by reference in its entirety.

Nanoparticles include inorganic nanoparticle carriers, polymeric nanoparticle carriers, and biologically derived nanoparticle carriers. In some embodiments, a broad-spectrum, pan-COV immunogenic composition disclosed herein is formulated with an inorganic nanoparticle carrier. In aspects of these embodiments, a broad-spectrum, pan-COV immunogenic composition disclosed herein is formulated with an inorganic nanoparticle carrier. In aspects of these embodiments, a broad-spectrum, pan-COV immunogenic composition disclosed herein is formulated with an inorganic nanoparticle carrier. Inorganic nanoparticle carriers include gold nanoparticles (AuNP), silver nanoparticles (AgNP), iron oxide nanoparticles, silica nanoparticles, aluminium nanoparticles, and calcium phosphate nanoparticles.

In some embodiments, a broad-spectrum, pan-COV immunogenic composition disclosed herein is formulated with a polymeric nanoparticle carrier. In aspects of these embodiments, a broad-spectrum, pan-COV immunogenic composition disclosed herein is formulated with a polymeric nanoparticle carrier. In aspects of these embodiments, a broad-spectrum, pan-COV immunogenic composition disclosed herein is formulated with a polymeric nanoparticle carrier. Polymeric nanoparticle carriers comprise polymers having a molecular weight between, for example, 5,000-200,000 Da, such as, e.g. between 10,000-100,000 Da, 20,000-70,000 Da, 40,000-50,000 Da. Non-limiting examples of polymer useful for polymeric nanoparticles include poly(D,L-lactide-co-glycolide) (PLGA), poly(lactic acid) (PLA), poly(γ-glutamic acid) (γ-PGA), poly(ethylene glycol) (PEG), poly(styrene), poly(N-vinylpyrrolidone), poly(ahydroxy acids), poly(hydroxy butyric acids), poly(lactones), including poly(caprolactones), poly(dioxanones), poly(valerolactone), poly(orthoesters), poly(anhydrides), poly(cyanoacrylates), tyrosine-derived poly(carbonates), poly(vinyl-pyrrolidinones) or poly(ester-amides), chitosan and dextran, or combinations thereof. Polymeric nanoparticle carriers and methods and uses of polymeric nanoparticle carriers are described in, e.g., U.S. Pat. No. 9,192,661, the content of each of which is hereby incorporated by reference in its entirety.

In some embodiments, a broad-spectrum, pan-COV immunogenic composition disclosed herein is formulated with a biologically derived nanoparticle carrier. In aspects of these embodiments, a broad-spectrum, pan-COV immunogenic composition disclosed herein is formulated with a biologically derived nanoparticle carrier. In aspects of these embodiments, a broad-spectrum, pan-COV immunogenic composition disclosed herein is formulated with a biologically derived nanoparticle carrier. Biologically derived nanoparticle carriers include, without limitation, lipid nanoparticle (LNP) carriers, nanolipoprotein (NLP) carriers, virus carriers, virus-like particle (VLP) carriers, virosome carriers, synthetic virus like particle (SVLP) carriers, self-assembling protein nanoparticle (SAPN) carriers, carbon nanotube (CNT) carriers, and peptide carriers.

In some embodiments, a broad-spectrum, pan-COV immunogenic composition disclosed herein is formulated with a lipid nanoparticle (LNP) carrier. In aspects of these embodiments, a broad-spectrum, pan-COV immunogenic composition disclosed herein is formulated with a lipid nanoparticle carrier. In aspects of these embodiments, a broad-spectrum, pan-COV immunogenic composition disclosed herein is formulated with a lipid nanoparticle carrier. LNP carriers are liposome-like structures that encapsulate one or more COV antigens disclosed herein. LNP carriers can comprise lipids that are saturated or unsaturated and be formed from a single lipid or from a mixture of lipids. LNP carriers are composed primarily of cationic lipids along with other lipid ingredients, such as, e.g., neutral phospholipid molecules belonging to the phosphatidylcholine (PC) class and sterols, such as cholesterol as well as a PEGylated phospholipid, a polyethylene glycol (PEG) polymer covalently attached to the head-group of a phospholipid. LNP carriers can also incorporates PLA or PGLA polymers within a lipid monolayer which facilitate controlled drug release. A LNP carrier may comprise (i) a cationic lipid, (ii) a mixture of a cationic lipid and a sterol, (iii) a mixture of a cationic lipid and a neutral phospholipid, (iv) a mixture of a cationic lipid, sterol, and a PEGylated phospholipid, (v) a mixture of a cationic lipid, a neutral phospholipid, and a PEGylated phospholipid, (vi) a mixture of a cationic lipid, sterol, a PEGylated phospholipid, and a polymer, (vii) a mixture of a cationic lipid, a neutral phospholipid, a PEGylated phospholipid, and a polymer. LNP carriers and methods and uses of LNP carriers are described in, e.g., U.S. Pat. Nos. 9,616,137, 9,629,922, 10,064,934, 10,207,010, 10,272,150, 10,273,269, 10,335,420, 10,485,885, the content of each of which is hereby incorporated by reference in its entirety.

In some embodiments, a lipid nanoparticle carrier disclosed herein comprises at least one lipid. In aspects of these embodiments, the at least one lipid includes comprise an ionizable lipid. An ionizable lipid refers to a lipid comprising one or more charged moieties. In some embodiments, an ionizable lipid may be positively charged (cationic lipid) or negatively charged (anionic lipid). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

In some embodiments, a lipid nanoparticle carrier disclosed herein comprises a cationic lipid. In aspects of these embodiments, a cationic lipid includes 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 1,2-dilinolenyloxy-N,N-dimethyl-3-aminopropane (DLen-DMA), 2,2-dilinoleyl-4-dimethylaminomethyl-[1,3]-dioxolane (DLin-K-DMA), heptatriaconta-6,9,28,31-tetraen-19-yl-4-(dimethylamino)butanoate (DLin-MC3-DMA), 2,2-dilinoleyl-4-(2-dimethylaminoethyl)[1,3]-dioxolane (DLin-KC2-DMA), di((Z)-non-2-en-1-yl)9-((4-(dimethylamino)butanoyl)oxy)heptadecanedioate (L319), (13Z,165Z)—N,N-dimethyl-3-nonydocosa-13-16-dien-1-amine (L608), 2-amino-3-[(9Z,12Z)-octadeca-9,12-dien-1-yloxy]-2-1{[(9Z,2Z)-octadeca-9,1-2-dien-1-yloxy]methyl}propan-1-ol, 2-amino-3-[(9Z)-octadec-9-en-1-yloxy]-2-{[(9Z)-octadec-9-en-1-yloxy]methyl}propan-1-ol, 2-amino-3-[(9Z,12Z)-octadeca-9,12-dien-1-yloxy]-2-[(octyloxy)methyl]propa-n-1-ol, 2-(dimethylamino)-3-[(9Z,12Z)-octadeca-9,12-dien-1-yloxy]-2-{[(9Z,12Z)-octadeca-9,12-dien-1-yloxy]methyl}propan-1-ol, 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-Clin-DMA), (2R)-2-({8-[(3β)-cholest-5-en-3-yloxy]octyl}oxy)-N,N-dimethyl-3-[(9Z-,12Z)-octadeca-9,12-dien-1-yloxy]propan-1-amine (Octyl-Clin-DMA (2R)), (2S)-2-({8-[(3β)-cholest-5-en-3-yloxy]octyl}oxy)-N,N-dimethyl-3-[(9Z-,12Z)-octadeca-9,12-dien-1-yloxy]propan-1-amine (Octyl-Clin-DMA (2S)), 98N12-5, C12-200, dioleoyl trimethylammonium propane (DOTAP), DSDMA, 1,2-dioleyloxy-N,N-dimethyl-3-aminopropane (DODMA), any pharmaceutically acceptable salt thereof, any stereoisomer thereof, or any combination thereof.

In some embodiments, a lipid nanoparticle carrier comprises an ionizable cationic lipid disclosed herein and further comprise a neutral lipid, a sterol, a phospholipid, and/or a polyethylene glycol (PEG)-modified lipid. In aspects of these embodiments, a neutral lipid includes DSPC, DPPC, POPC, DOPE and/or SM. In aspects of these embodiments, a sterol includes cholesterol. In aspects of these embodiments, a neutral lipid includes DSPC, DPPC, POPC, DOPE and/or SM. In aspects of these embodiments, a sterol includes cholesterol. In aspects of these embodiments, a molecule capable of reducing particle aggregation. In aspects of these embodiments, a phospholipid includes a phosphatidylethanolamine, a phosphatidylcholine, a phosphatidylserine, and/or a phosphatidylglycerol, any pharmaceutically acceptable salt thereof, any stereoisomer thereof, or any combination thereof. In aspects of these embodiments, a PEG-modified lipid (PEGylated lipids) include a PEG-modified phosphatidylethanolamine, a PEG-modified phosphatidic acid, a PEG-ceramide conjugate (e.g., PEG-CerC₁₄ or PEG-CerC₂₀), a PEG-modified dialkylamines and/or a PEG-modified 1,2-diacyloxypropan-3-amine, any pharmaceutically acceptable salt thereof, any stereoisomer thereof, or any combination thereof. In aspects of these embodiments, a PEG-modified lipid (PEGylated lipids) includes R-3-[(Ω-methoxy-poly(ethyleneglycol)2000)carbamoyl)]-1,2-dimyristy-loxypropyl-3-amine) (PEG-c-DOMG or PEG-DOMG), PEG-diacylglycamide (PEG-DAG), 1,2-dimyristoyl-sn-glycerol methoxypolyethylene glycol (PEG-DMG), 1,2-Dipalmitoyl-sn-glycerol, methoxypolyethylene glycol (PEG-DPG), 1,2-Distearoyl-sn-glycerol, methoxypolyethylene glycol (PEG-DSG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-dipalmitoyl phosphatidylethanolamine (PEG-DPPE), PEG-DLPE, PEG-DMPE, PEG-DPPC, PEG-1,2-dimyristyloxlpropyl-3-amine (PEG-c-DMA), any pharmaceutically acceptable salt thereof, any stereoisomer thereof, or any combination thereof.

In some embodiments, a lipid nanoparticle carrier has a molar ratio of about 20-60% cationic lipid:about 5-25% phospholipid:about 25-55% sterol, and about 0.5-15% PEG-modified lipid. In some embodiments, a lipid nanoparticle carrier has a molar ratio of about 50% cationic lipid, about 10% phospholipid, about 38.5% sterol, and about 1.5% PEG-modified lipid. In some embodiments, a lipid nanoparticle carrier has a molar ratio of about 55% cationic lipid, about 10% phospholipid, about 32.5% sterol, and about 2.5% PEG-modified lipid, and. In some embodiments, a lipid nanoparticle carrier has a molar ratio of 50:38.5:10:1.5 of cationic lipid:cholesterol:DSPC:PEG-DMG.

In some embodiments, a lipid nanoparticle carrier has a molar ratio of about 20-60% cationic lipid:about 5-25% neutral lipid:about 25-55% sterol, and about 0.5-15% PEG-modified lipid. In some embodiments, a lipid nanoparticle carrier has a molar ratio of about 20-60% DLin-KC2-DMA, DLin-MC3-DMA, or L319:about 5-25% DSPC, DPPC, POPC, DOPE or SM, about 25-55% cholesterol, and about 0.5-15% PEG-modified lipid.

In some embodiments, a lipid nanoparticle carrier disclosed herein comprises a lipid-polycation complex, referred to as a cationic lipid nanoparticle. In aspects of these embodiments, a polycation may include a cationic peptide or a polypeptide such as, but not limited to, polylysine, polyornithine and/or polyarginine.

In some embodiments, a broad-spectrum, pan-COV immunogenic composition disclosed herein is formulated with a nanolipoprotein carrier. In aspects of these embodiments, a broad-spectrum, pan-COV immunogenic composition disclosed herein is formulated with a nanolipoprotein (NLP) carrier. In aspects of these embodiments, a broad-spectrum, pan-COV immunogenic composition disclosed herein is formulated with a nanolipoprotein carrier. NLP carriers are high-density lipoprotein (HDL) mimetics, comprised of discoidal lipid bilayer stabilized by apolipoproteins, that are readily functionalized to provide a versatile delivery platform. NLP carriers and methods and uses of NLP carriers are described in, e.g., U.S. Pat. Nos. 8,907,061, 9,458,191, and 10,568,898, and U.S. Patent Application Publications 2018/0186860, 2018/0318218, 2019/0142752, and 2019/0307692, the content of each of which is hereby incorporated by reference in its entirety.

In some embodiments, a broad-spectrum, pan-COV immunogenic composition disclosed herein is formulated with a virus carrier. In aspects of these embodiments, a mucleotide sequence encoding one or more COV antigens disclosed herein is subcloned into a viral expression vector to create a viral construct. For delivery by a viral construct, a range of viral vectors are known in the art (including gene therapy vectors) and any suitable vector may be used for transient expression, episomal expression (persistent but not integrated into the host genome) or integration of the encoding nucleic acid into cells of the recipient (e.g., liver cells and/or cells of the haematopoietic system). Suitable vectors and delivery systems include viral DNA and/or RNA, such as systems based on adenoviruses, adeno-associated viruses, alphaviruses, herpesviruses, lentiviruses, poxviruses, retroviruses, vaccinia viruses, or hybrids containing elements of more than one virus. Such viral constructs can then be induced to produce recombinant viruses used to transduce a target T-cell population which is expanded before infusion into an individual. Alternatively, a viral construct can be expressed in vivo by the recipient cells of an individual, providing an in vivo source of the polypeptide antigen, suitable for provision of long term protection against a species, strain, genotype, subgenotype, serotype or genetic/antigenic lineage of Coronavirus or a component comprising a species, strain, genotype, subgenotype, serotype or genetic/antigenic lineage of Coronavirus.

In some embodiments, a broad-spectrum, pan-COV immunogenic composition disclosed herein is formulated with a virus-like particle carrier. In aspects of these embodiments, a broad-spectrum, pan-COV immunogenic composition disclosed herein is formulated with a virus-like particle carrier. In aspects of these embodiments, a broad-spectrum, pan-COV immunogenic composition disclosed herein is formulated with a virus-like particle carrier. VLP carriers are molecules that closely resemble viruses but are non-infectious because they contain no viral genetic material. They can be naturally occurring or synthesized through the individual expression of viral structural proteins, which can then self-assemble into the virus-like structure. Combinations of structural capsid proteins from different viruses can be used to create recombinant VLP carriers. Non-limiting examples of VLP carriers include those derived from components of icosahedral or spherical plant viruses like cowpea mosaic virus (CPMV), cowpea chlorotic mottle virus (CCMV), and alfalfa mosaic virus (AMV), those derived from components of rod-shaped plant viruses like tobacco mosaic virus (TMV) and potato virus X (PVX), and those derived from components of bacteriophages like bacteriophage AP205 and bacteriophage Qβ. VLP carriers and methods and uses of VLP carriers are described in, e.g., U.S. Pat. Nos. 6,153,201, 7,641,896, 7,951,384, 7,959,928, 8,062,642, 8,183,044, 8,282,940, 8,460,676, 8,735,561, 8,758,762, 8,795,682, 8,889,147, 9,045,727, 9,517,261, 9,580,474, 9,782,471, 10,080,798, 10,358,656, 10,428,117, 10,526,376, 10,532,077, the content of each of which is hereby incorporated by reference in its entirety.

In some embodiments, a broad-spectrum, pan-COV immunogenic composition disclosed herein is formulated with a self-assembling protein nanoparticle carrier. In aspects of these embodiments, a broad-spectrum, pan-COV immunogenic composition disclosed herein is formulated with a self-assembling protein nanoparticle carrier. In aspects of these embodiments, a broad-spectrum, pan-COV immunogenic composition disclosed herein is formulated with a self-assembling protein nanoparticle carrier. A self-assembling protein nanoparticle (SAPN) carrier disclosed herein includes, without limitation, caged protein nanoparticle carriers, proteosome nanoparticle carriers, cochleate nanoparticle carriers, and dendrimer nanoparticle carriers.

In some embodiments, a broad-spectrum, pan-COV immunogenic composition disclosed herein is formulated with a caged protein nanoparticle carrier. In aspects of these embodiments, a broad-spectrum, pan-COV immunogenic composition disclosed herein is formulated with a caged protein nanoparticle carrier. In aspects of these embodiments, a broad-spectrum, pan-COV immunogenic composition disclosed herein is formulated with a caged protein nanoparticle carrier. Caged protein nanoparticle carriers comprise self-assembly polypeptides and subunits thereof that encapsulate one or more COV antigens disclosed herein. Non-limiting examples of caged protein carriers include ferritin nanoparticles, encapsulin nanoparticles, sulfur oxygenase reductase nanoparticles, lumazine synthase nanoparticles, small heat-shock proteins (sHSP) nanoparticles like HSP 70 nanoparticles, HSP 96 nanoparticles, HSP 110 nanoparticles, dihydrolipoamide acetyltransferase (E2) nanoparticles, or vault protein nanoparticles. Caged protein nanoparticle carriers and methods and uses of caged protein nanoparticle carriers are described in, e.g., U.S. Pat. Nos. 6,156,879, 6,555,347, 7,132,109, 7,297,535, 9,441,019, 10,137,190, 10,420,830, 10,744,199, 10,676,534, the content of each of which is hereby incorporated by reference in its entirety.

In some embodiments, a broad-spectrum, pan-COV immunogenic composition disclosed herein is formulated with a proteosome nanoparticle carrier. In aspects of these embodiments, a broad-spectrum, pan-COV immunogenic composition disclosed herein is formulated with a proteosome nanoparticle carrier. In aspects of these embodiments, a broad-spectrum, pan-COV immunogenic composition disclosed herein is formulated with a proteosome nanoparticle carrier. Proteosome nanoparticle carriers comprises a purified preparation of outer membrane proteins of meningococci and similar preparations from other bacteria. These proteins are highly hydrophobic, reflecting their role as transmembrane proteins and porins. Due to their hydrophobic protein-protein interactions, when appropriately isolated, the proteins form multi-molecular structures consisting of about 60-100 nm diameter whole or fragmented membrane vesicles. This liposome-like physical state allows the proteosome adjuvant to act as a nanoparticle carrier and also to act as an adjuvant. Proteosome nanoparticle carriers are useful for intranasal administration. Proteosome nanoparticle carriers and methods and uses of proteosome nanoparticle carriers are described in, e.g., U.S. Pat. Nos. 5,726,292, 6,476,201, 6,743,900, 6,803,042, 7,255,867, 7,399,840, 8,173,140, and 9,433,672, the content of each of which is hereby incorporated by reference in its entirety.

In some embodiments, a broad-spectrum, pan-COV immunogenic composition disclosed herein is formulated with a cochleate nanoparticle carrier. In aspects of these embodiments, a broad-spectrum, pan-COV immunogenic composition disclosed herein is formulated with a cochleate nanoparticle carrier. In aspects of these embodiments, a broad-spectrum, pan-COV immunogenic composition disclosed herein is formulated with a cochleate nanoparticle carrier. Cochleate nanoparticle carriers are stable phospholipid-cation precipitates that generally form alternating cationic and lipid bilayer sheets. The bilayer structure of cochleates provides protection from degradation for associated, or “encochleated,” molecules. Since the entire cochleate structure is a series of solid layers, components within the interior of the cochleate structure remain substantially intact, even though the outer layers of the cochleate may be exposed to harsh environmental conditions or enzymes. Cochleate nanoparticle carriers and methods and uses of cochleate nanoparticle carriers are described in, e.g., U.S. Pat. Nos. 5,840,707, 5,994,318, 6,153,217, 6,340,591, 6,592,894, 8,598,138, 8,642,073, 9,370,572, 9,775,907, 9,974,745, and 10,716,860, the content of each of which is hereby incorporated by reference in its entirety.

In some embodiments, a broad-spectrum, pan-COV immunogenic composition disclosed herein is formulated with a dendrimer nanoparticle carrier. In aspects of these embodiments, a broad-spectrum, pan-COV immunogenic composition disclosed herein is formulated with a dendrimer nanoparticle carrier. In aspects of these embodiments, a broad-spectrum, pan-COV immunogenic composition disclosed herein is formulated with a dendrimer nanoparticle carrier. Dendrimer nanoparticle carriers can function by encapsulating one or more COV antigens disclosed herein, conjugating one or more COV antigens disclosed herein to their surface via a linker, adsorbing one or more COV antigens disclosed herein to their surface, or be admixed with one or more COV antigens disclosed herein. Dendrimer nanoparticle carriers are characterized by a three-dimensional, star-shaped, branched macromolecular network having interior and the exterior layers. Dendrimer nanoparticle carriers possess low polydispersity index, biocompatibility and good water solubility. Non-limiting examples of dendrimer carriers include polyamidoamine (PAMAM), poly-1-lysine (PLL), poly (propylene imine) (PPI), diaminobutane amine polypropylenimine tetramine (DAB-Am 4), polypropylamine (POPAM), polylysine, polyester, iptycene, aliphatic poly(ether), and/or aromatic polyether dendrimers. Dendrimer nanoparticle carriers and methods and uses of dendrimer nanoparticle carriers are described in, e.g., U.S. Pat. Nos. 5,661,025, 5,977,084, 5,990,089, 8,252,834, 8,287,849, 8,945,508, 9,345,781, 9,687,561, 10,548,959, the content of each of which is hereby incorporated by reference in its entirety.

In some embodiments, a broad-spectrum, pan-COV immunogenic composition disclosed herein is formulated with a carbon nanotube carrier. In aspects of these embodiments, a broad-spectrum, pan-COV immunogenic composition disclosed herein is formulated with a carbon nanotube carrier. In aspects of these embodiments, a broad-spectrum, pan-COV immunogenic composition disclosed herein is formulated with a carbon nanotube carrier. Carbon nanotube (CNT, or carbon nanofibers) carriers can function by encapsulating one or more COV antigens disclosed herein, conjugating one or more COV antigens disclosed herein to their surface via a linker, adsorbing one or more COV antigens disclosed herein to their surface, or be admixed with one or more COV antigens disclosed herein. CNT carriers are sheets of graphene folded into a tube shape with diameters typically measured in nanometers. CNT can occur in a single layer, single-walled carbon nanotubes (SWNT), or in multiple layers, multi-walled carbon nanotubes (MWNT). CNT carriers and methods and uses of CNT carriers are described in, e.g., U.S. Pat. Nos. 7,858,648, 8,536,324, 8,540,965, 8,658,178, 9,737,593, and 10,485,856, the content of each of which is hereby incorporated by reference in its entirety.

In some embodiments, a broad-spectrum, pan-COV immunogenic composition disclosed herein is formulated with a peptide carrier. In aspects of these embodiments, a broad-spectrum, pan-COV immunogenic composition disclosed herein is formulated with a peptide carrier. In aspects of these embodiments, a broad-spectrum, pan-COV immunogenic composition disclosed herein is formulated with a peptide carrier. Peptide carriers can function in destabilizing cell membranes in order to increase cellular uptake and facilitate delivery of a broad-spectrum, pan-COV immunogenic composition disclosed herein. Non-limiting examples of peptide carriers include the pore-forming peptides, membrane-permeabilizing proteins, and membrane destabilizing peptides (or cell penetrating peptides). Membrane destabilizing peptides, include, without limitation, aurein, GALA, mast 21, TP2, ppTG20, Vpr52-96, JTS-1, LAH (including LAH1, LAH2, LAH3, LAH4, and LAH5), CADY, KALA, RAWA, and RALA. Peptide carriers and methods and uses of peptide carriers are described in, e.g., U.S. Pat. Nos. 7,906,484, 8,728,793, 9,744,244, 10,188,744, 10,407,469, and 10,500,287 the content of each of which is hereby incorporated by reference in its entirety.

A broad-spectrum, pan-COV immunogenic composition disclosed herein may further comprise one or more pharmaceutical components, including, without limitation, solvents, dispersion media, buffers, preservatives, tonicity adjusters, salts, antioxidants, osmolality adjusting agents, physiological substances, pharmacological substances, bulking agents, emulsifying agents, wetting agents, sweetening or flavouring agents, and the like. Various buffers and means for adjusting pH can be used to prepare a pharmaceutical composition disclosed in the present specification, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition, such as, e.g., PURITE® and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition.

Formulations of a broad-spectrum, pan-COV immunogenic composition described herein may be prepared by any method known or hereafter developed in the art of pharmacology. In general, such preparatory methods include the step of bringing the active ingredient (such as, e.g., one or more COV peptide antigens disclosed herein and/or one or more nucleotide sequences encoding one or more COV peptide antigens disclosed herein) into association with a carrier and/or one or more other pharmaceutical components, and then, if necessary and/or desirable, dividing, shaping and/or packaging the product into a desired single- or multi-dose unit. Relative amounts of the active ingredient, the pharmaceutically acceptable carrier, and/or any additional pharmaceutical components in a COV vaccine composition described herein will vary, depending upon the identity, size, and/or condition of the individual treated and further depending upon the route by which the COV vaccine composition described herein is to be administered. By way of example, a COV vaccine composition described herein may comprise between 0.1% and 100%, e.g., between 0.5 and 50%, between 1-30%, between 5-80%, at least 80% (w/w) active ingredient.

Aspects of the present specification disclose methods of preventing a Coronavirinae-based infection by administering a broad-spectrum, pan-COV immunogenic composition disclosed herein to an individual. In some embodiments, methods of preventing a Coronavirinae-based infection or pathology comprise administering a broad-spectrum, pan-COV immunogenic composition to an individual. In some embodiments, methods of treating a Coronavirinae-based infection or pathology comprise administering a broad-spectrum, pan-COV immunogenic composition to an individual.

Aspects of the present specification disclose also include a broad-spectrum, pan-COV immunogenic composition disclosed herein for use in preventing a Coronavirinae-based infection or pathology. In some embodiments, the present specification discloses a peptide-based broad-spectrum, pan-COV immunogenic composition disclosed herein for use in preventing a Coronavirinae-based infection or pathology. In some embodiments, the present specification discloses a recombinant protein-based broad-spectrum, pan-COV immunogenic composition disclosed herein for use in preventing a Coronavirinae-based infection or pathology. In some embodiments, the present specification discloses a nucleotide-based broad-spectrum, pan-COV immunogenic composition disclosed herein for use in preventing a Coronavirinae-based infection or pathology.

Aspects of the present specification disclose also include use of a broad-spectrum, pan-COV immunogenic composition disclosed herein in treating a Coronavirinae-based infection or pathology. In some embodiments, the present specification discloses use of a peptide-based broad-spectrum, pan-COV immunogenic composition disclosed herein in treating a Coronavirinae-based infection or pathology. In some embodiments, the present specification discloses use of a recombinant protein-based broad-spectrum, pan-COV immunogenic composition disclosed herein in treating a Coronavirinae-based infection or pathology. In some embodiments, the present specification discloses use of a nucleotide-based broad-spectrum, pan-COV immunogenic composition disclosed herein in treating a Coronavirinae-based infection or pathology.

Aspects of the present specification disclose also include use of a broad-spectrum, pan-COV immunogenic composition disclosed herein in the manufacture of a medicament for treating a Coronavirinae-based infection or pathology. In some embodiments, the present specification discloses use of a peptide-based broad-spectrum, pan-COV immunogenic composition disclosed herein in the manufacture of a medicament for treating a Coronavirinae-based infection or pathology. In some embodiments, the present specification discloses use of a recombinant protein-based broad-spectrum, pan-COV immunogenic composition disclosed herein in the manufacture of a medicament for treating a Coronavirinae-based infection or pathology. In some embodiments, the present specification discloses use of a nucleotide-based broad-spectrum, pan-COV immunogenic composition disclosed herein in the manufacture of a medicament for treating a Coronavirinae-based infection or pathology.

Aspects of the present specification disclose methods of creating, maintaining, or restoring antigenic memory to one or more Coronavirus species, strains, genotypes, subgenotypes, serotypes or genetic/antigenic lineages in an individual or population of individuals comprises administering a broad-spectrum, pan-COV immunogenic composition disclosed herein. In some embodiments, methods of creating, maintaining, or restoring antigenic memory to one or more Coronavirus species, strains, genotypes, subgenotypes, serotypes or genetic/antigenic lineages in an individual or population of individuals comprises administering a peptide-based broad-spectrum, pan-COV immunogenic composition disclosed herein to an individual. In some embodiments, methods of creating, maintaining or restoring antigenic memory to one or more COV strains/groups in an individual or population of individuals comprises administering a recombinant protein-based broad-spectrum, pan-COV immunogenic composition disclosed herein to an individual. In some embodiments, methods of creating, maintaining or restoring antigenic memory to one or more Coronavirus species, strains, genotypes, subgenotypes, serotypes or genetic/antigenic lineages in an individual or population of individuals comprises administering a nucleotide-based broad-spectrum, pan-COV immunogenic composition disclosed herein to an individual.

Aspects of the present specification also disclose a broad-spectrum, pan-COV immunogenic composition disclosed herein for use in creating, maintaining, or restoring antigenic memory to one or more Coronavirus species, strains, genotypes, subgenotypes, serotypes or genetic/antigenic lineages in an individual or population of individuals. In some embodiments, the present specification discloses a peptide-based broad-spectrum, pan-COV immunogenic composition disclosed herein for use in creating, maintaining, or restoring antigenic memory to one or more Coronavirus species, strains, genotypes, subgenotypes, serotypes or genetic/antigenic lineages in an individual or population of individuals. In some embodiments, the present specification discloses a recombinant protein-based broad-spectrum, pan-COV immunogenic composition disclosed herein for use in creating, maintaining or restoring antigenic memory to one or more Coronavirus species, strains, genotypes, subgenotypes, serotypes or genetic/antigenic lineages in an individual or population of individuals. In some embodiments, the present specification discloses a nucleotide-based broad-spectrum, pan-COV immunogenic composition disclosed herein for use in creating, maintaining or restoring antigenic memory to one or more Coronavirus species, strains, genotypes, subgenotypes, serotypes or genetic/antigenic lineages in an individual or population of individuals.

Aspects of the present specification also disclose use of a broad-spectrum, pan-COV immunogenic composition disclosed herein in creating, maintaining or restoring antigenic memory to one or more Coronavirus species, strains, genotypes, subgenotypes, serotypes or genetic/antigenic lineages in an individual or population of individuals. In some embodiments, the present specification discloses use of a peptide-based broad-spectrum, pan-COV immunogenic composition disclosed herein in creating, maintaining or restoring antigenic memory to one or more Coronavirus species, strains, genotypes, subgenotypes, serotypes or genetic/antigenic lineages in an individual or population of individuals. In some embodiments, the present specification discloses use of a recombinant protein-based broad-spectrum, pan-COV immunogenic composition disclosed herein in creating, maintaining or restoring antigenic memory to one or more Coronavirus species, strains, genotypes, subgenotypes, serotypes or genetic/antigenic lineages in an individual or population of individuals. In some embodiments, the present specification discloses use of a nucleotide-based broad-spectrum, pan-COV immunogenic composition disclosed herein in creating, maintaining or restoring antigenic memory to one or more Coronavirus species, strains, genotypes, subgenotypes, serotypes or genetic/antigenic lineages in an individual or population of individuals.

Aspects of the present specification also disclose use of a broad-spectrum, pan-COV immunogenic composition disclosed herein in the manufacture of a medicament for creating, maintaining or restoring antigenic memory to one or more Coronavirus species, strains, genotypes, subgenotypes, serotypes or genetic/antigenic lineages in an individual or population of individuals. In some embodiments, the present specification discloses use of a peptide-based broad-spectrum, pan-COV immunogenic composition disclosed herein in the manufacture of a medicament for creating, maintaining, or restoring antigenic memory to one or more Coronavirus species, strains, genotypes, subgenotypes, serotypes or genetic/antigenic lineages in an individual or population of individuals. In some embodiments, the present specification discloses use of a recombinant protein-based broad-spectrum, pan-COV immunogenic composition disclosed herein in the manufacture of a medicament for creating, maintaining, or restoring antigenic memory to one or more Coronavirus species, strains, genotypes, subgenotypes, serotypes or genetic/antigenic lineages in an individual or population of individuals. In some embodiments, the present specification discloses use of a nucleotide-based broad-spectrum, pan-COV immunogenic composition disclosed herein in the manufacture of a medicament for creating, maintaining, or restoring antigenic memory to one or more Coronavirus species, strains, genotypes, subgenotypes, serotypes or genetic/antigenic lineages in an individual or population of individuals.

The methods and uses disclosed herein include therapeutic (following COV infection) and prophylactic (prior to COV exposure, infection or pathology) administration of a broad-spectrum, pan-COV immunogenic composition disclosed herein as part of an active immunization scheme to healthy individuals or early in infection during the incubation phase or during active infection after onset of symptoms. In some embodiments, the amount of a broad-spectrum, pan-COV immunogenic composition provided to a cell, a tissue or a subject may be an amount effective for immune prophylaxis. For example, therapeutic and prophylactic methods and uses of treating an individual for a Coronavirinae-based infection include treatment of an individual having or at risk of having a Coronavirinae-based infection or pathology, treating an individual with a Coronavirinae-based infection, and methods of protecting an individual from a Coronavirinae-based infection, to reduce, suppress or eliminate the probability of a Coronavirinae-based infection in an individual, to reduce, suppress or eliminate susceptibility of an individual to a Coronavirinae-based infection, to reduce, suppress or eliminate a Coronavirinae-based infection in an individual, to reduce, suppress or eliminate an symptomology, a morbidity and/or mortality, or to reduce, suppress or eliminate transmission of a COV from an infected individual to an uninfected individual. Such methods and uses include administering an immunogenic composition disclosed herein to therapeutically or prophylactically treat (vaccinate or immunize) an individual having or at risk of having a Coronavirinae-based infection or pathology. Accordingly, methods and uses disclosed herein can treat the COV infection or pathology, or provide the individual with protection from infection (e.g., prophylactic protection).

In some embodiments, a method or use of treating a Coronavirinae-based infection or pathology comprises administering to an individual in need thereof a broad-spectrum, pan-COV immunogenic composition disclosed herein in an amount sufficient to reduce one or more physiological conditions or symptom associated with a Coronavirinae-based infection or pathology, thereby treating the Coronavirinae-based infection or pathology. In aspects of these embodiments, a broad-spectrum, pan-COV immunogenic composition comprises one or more COV peptide antigens disclosed herein. In aspects of these embodiments, a broad-spectrum, pan-COV immunogenic composition comprises a recombinant protein comprising one or more COV peptide antigens disclosed herein. In other aspects of these embodiments, a broad-spectrum, pan-COV immunogenic composition comprises one or more nucleotide sequences, each nucleotide sequence encoding a polypeptide including one or more COV peptide antigens disclosed herein.

In some embodiments, use of a broad-spectrum, pan-COV immunogenic composition disclosed herein treats a Coronavirinae-based infection or pathology by reducing one or more physiological conditions or symptom associated with a Coronavirinae-based infection or pathology. In some embodiments, administration of a COV peptide antigen or an immunogenic composition disclosed herein is in an amount sufficient to reduce one or more physiological conditions or symptom associated with a Coronavirinae-based infection or pathology, thereby treating the Coronavirinae-based infection or pathology. In some embodiments, administration of a COV antigen or an immunogenic composition disclosed herein is in an amount sufficient to increase, induce, enhance, augment, promote or stimulate clearance or removal of a species, strain, genotype, subgenotype, serotype or genetic/antigenic lineage of a Coronavirus; or decrease, reduce, inhibit, suppress, prevent, control, or limit transmission of a species strain, genotype, subgenotype, serotype or genetic/antigenic lineage of Coronavirus to another individual. In aspects of these embodiments, an immunogenic composition comprises one or more COV peptide antigens disclosed herein. In aspects of these embodiments, an immunogenic composition comprises a recombinant protein comprising one or more COV peptide antigens disclosed herein. In other aspects of these embodiments, an immunogenic composition comprises one or more nucleotide sequences, each nucleotide sequence encoding a polypeptide including one or more COV peptide antigens disclosed herein.

In some embodiments, a method of treating a Coronavirinae-based infection or pathology comprises administering to an individual in need thereof a COV peptide antigen or immunogenic composition disclosed herein in an amount sufficient to immunize or vaccinate the individual against a Coronavirinae-based infection or pathology, thereby treating the Coronavirinae-based infection or pathology. In some embodiments, administration of an immunogenic composition disclosed herein is in an amount sufficient to increase, induce, enhance, augment, promote or stimulate an immune response against a species, strain, genotype, subgenotype, serotype or genetic/antigenic lineage of Coronavirus or component comprising a species, strain, genotype, subgenotype, serotype or genetic/antigenic lineage of Coronavirus. In some embodiments, administration of an immunogenic composition disclosed herein is in an amount sufficient to increase, induce, enhance, augment, promote or stimulate clearance or removal of a species, strain, genotype, subgenotype, serotype or genetic/antigenic lineage of Coronavirus; or decrease, reduce, inhibit, suppress, prevent, control, or limit transmission of a species, strain, genotype, subgenotype, serotype or genetic/antigenic lineage of Coronavirus to another individual. In aspects of these embodiments, an immunogenic composition comprises one or more COV peptide antigens disclosed herein. In other aspects of these embodiments, an immunogenic composition comprises one or more nucleotide sequences, each nucleotide sequence encoding a polypeptide including one or more COV peptide antigens disclosed herein.

In some embodiments, a method of treating a Coronavirinae-based infection or pathology comprises administering to an individual in need thereof a COV antigen or immunogenic composition disclosed herein in an amount sufficient to protect the individual against a Coronavirinae-based infection or pathology, thereby treating the Coronavirinae-based infection or pathology. In some embodiments, administration of an immunogenic composition disclosed herein is in an amount sufficient to immunize or vaccinate the individual against the COV infection or pathology, or reduce, decrease, limit, control or inhibit susceptibility to a Coronavirinae-based infection or pathology. In aspects of these embodiments, an immunogenic composition comprises one or more COV peptide antigens disclosed herein. In other aspects of these embodiments, an immunogenic composition comprises one or more nucleotide sequences, each nucleotide sequence encoding a polypeptide including one or more COV peptide antigens disclosed herein.

In some embodiments, a broad-spectrum, pan-COV immunogenic composition disclosed herein is used to treat a Coronavirinae-based infection or pathology. In an aspect of this embodiment, use of a broad-spectrum, pan-COV immunogenic composition disclosed herein is in an amount sufficient to immunize or vaccinate the individual against the COV infection or pathology. In aspects of these embodiments, a broad-spectrum, pan-COV immunogenic composition comprises one or more COV peptide antigens disclosed herein. In other aspects of these embodiments, a broad-spectrum, pan-COV immunogenic composition comprises one or more nucleotide sequences, each nucleotide sequence encoding a polypeptide including one or more COV peptide antigens disclosed herein.

A COV infection or pathology refers to any condition, infection, disease or disorder where a pathophysiology effect is due to the presence of members of a species, strain, genotype, subgenotype, serotype or genetic/antigenic lineage of Coronavirus or a component comprising a species, strain, genotype, subgenotype, serotype or genetic/antigenic lineage of Coronavirus.

In some embodiments, a Coronavirinae-based infection or pathology is an immunological deficiency or pathology in an individual that result from a Coronavirinae-based infection or pathology.

Aspects of the present invention provide, in part, an individual. An individual refers to any mammal including but not limited to a human. As such, a method disclosed herein is for human use. An individual is typically a human and a human can be a patient. Typically, any individual who is a candidate for a conventional broad-spectrum, pan-COV immunogenic composition procedure is a candidate for a method or use disclosed herein. In addition, the presently disclosed compositions and methods and uses may apply to individuals seeking prophylactic protection or therapeutic benefit against a Coronavirinae-based infection or pathology which may not be technically possible with existing broad-spectrum, pan-COV immunogenic compositions.

A method disclosed herein comprises a treatment for a Coronavirinae-based infection or pathology. A treatment comprises any therapeutic or beneficial effect, including any objective or individually measurable or detectable improvement or benefit provided to a particular individual. A therapeutic or beneficial effect can but need not be complete ablation of all or any particular adverse condition, symptom, disorder, illness, disease, infection, pathology, or complication caused by or associated with a Coronavirinae-based infection, proliferation, replication, or pathology. Thus, a satisfactory clinical endpoint is achieved when there is an incremental improvement or a partial reduction in an adverse condition, symptom, disorder, illness, disease, infection, pathology, or complication caused by or associated with a Coronavirinae-based infection, proliferation, replication, or pathology, or an inhibition, decrease, reduction, suppression, prevention, limit or control of worsening or progression of one or more conditions, adverse symptoms, disorders, illnesses, disease, infection, pathology, or complications caused by or associated with a Coronavirinae-based infection, proliferation, replication, or pathology over a short or long duration.

In aspects of this embodiment, a method of treatment or use disclosed herein may reduce, decrease, inhibit, limit, delay or prevent a Coronavirinae-based infection, proliferation, replication, or pathology. In other aspects of this embodiment, a method of treatment or use disclosed herein may reduce, decrease, suppress, limit, control or inhibit COV pathogen numbers or titer; reduce, decrease, suppress, limit, control or inhibit COV pathogen proliferation or replication; reduce, decrease, suppress, limit, control or inhibit the amount of a COV protein synthesized; or reduce, decrease, suppress, limit, control or inhibit the amount of a COV pathogen nucleic acid replicated. In yet other aspects of this embodiment, a method of treatment or use disclosed herein may decrease, reduce, inhibit, suppresses, prevent, control or limit one or more adverse conditions, symptoms, disorders, illnesses, disease, infections, pathologies, or complications caused by or associated with a Coronavirinae-based infection, proliferation or replication, or pathology. In still other aspects of this embodiment, a method of treatment or use disclosed herein may improve, accelerate, facilitate, enhance, augment, or hasten recovery of an individual from a Coronavirinae-based infection or pathology, or one or more adverse symptoms, disorders, illnesses, disease, infections, pathologies, or complications caused by or associated with COV infection, proliferation or replication, or pathology.

In other aspects of this embodiment, a method of treatment or use disclosed herein may stabilize a Coronavirinae-based infection, pathology, or an adverse condition, symptom, disorder, illness, infection, pathology, or complication caused by or associated with a Coronavirinae-based infection, proliferation, replication, or pathology. In yet other aspects of this embodiment, a method of treatment or use disclosed herein may decrease, reduce, inhibit, suppress, limit or control transmission of a Coronavirinae pathogen from an infected individual to an uninfected individual. In still other aspects of this embodiment, a method of treatment or use disclosed herein may reduce or eliminate the need, dosage frequency or amount of a concurrent or subsequent treatment such as another drug or other agent used for treating an individual having or at risk of having a Coronavirinae-based infection or pathology. For example, reducing an amount of an adjunct therapy, for example, a reduction or decrease of a treatment for a Coronavirinae-based infection or pathology, or a vaccination or immunization protocol is considered a beneficial effect. In addition, reducing or decreasing an amount of a COV antigen used for vaccination or immunization of an individual to provide protection to the individual is considered a beneficial effect.

Aspects of the present specification provide, in part, administering a broad-spectrum, pan-COV immunogenic composition disclosed herein. As used herein, the term “administering” refers to any delivery mechanism that provides a broad-spectrum, pan-COV immunogenic composition disclosed herein to an individual that potentially results in a clinically, therapeutically, or experimentally beneficial result. The actual delivery mechanism used to administer a broad-spectrum, pan-COV immunogenic composition disclosed herein to an individual can be determined by a person of ordinary skill in the art by taking into account factors, including, without limitation, the type of COV infection or pathology, the location of the Coronavirinae-based infection or pathology, the cause of the Coronavirinae-based infection or pathology, the severity of the Coronavirinae-based infection or pathology, the degree of relief desired for a Coronavirinae-based infection or pathology, the duration of relief desired for a Coronavirinae-based infection or pathology, the particular COV antigen and/or broad-spectrum, pan-COV immunogenic composition used, the rate of excretion of the particular antigen and/or broad-spectrum, pan-COV immunogenic composition used, the pharmacodynamics of the particular COV antigen and/or broad-spectrum, pan-COV immunogenic composition used, the nature of the other compounds to be included in the broad-spectrum, pan-COV immunogenic composition, the particular route of administration, the particular means of administration used, the particular target tissue, the particular target cell type, the particular characteristics, history and risk factors of the individual, such as, e.g., age, weight, general health and the like, or any combination thereof.

A broad-spectrum, pan-COV immunogenic composition disclosed herein can be administered to an individual using any route which results in a therapeutically effective outcome. Administration of a composition disclosed herein using a cellular uptake approach comprise a variety of enteral or parenteral approaches including, without limitation, oral administration in any acceptable form, such as, e.g., tablet, liquid, capsule, powder, an inhalable or the like; topical administration in any acceptable form, such as, e.g., drops, spray, creams, gels or ointments; respiratory administration in any acceptable form, such as, e.g., nasal inhalation and lung inhalation; intravascular administration in any acceptable form, such as, e.g., intravenous injection, intravenous infusion, intra-arterial injection, intra-arterial infusion and catheter instillation into the vasculature; peri- and intra-tissue administration in any acceptable form, such as, e.g., intraperitoneal injection, intramuscular injection, subcutaneous injection, subcutaneous infusion, intradermal injection, intraocular injection, retinal injection, sub-retinal injection or epidural injection; intravesicular administration in any acceptable form, such as, e.g., catheter instillation; and by placement device, such as, e.g., an implant, a patch, a pellet, a catheter, an osmotic pump, a suppository, a bioerodible delivery system, a non-bioerodible delivery system or another implanted extended or slow release system. An exemplary list of biodegradable polymers and methods of use are described in, e.g., Handbook of Biodegradable Polymers (Abraham J. Domb et al., eds., Overseas Publishers Association, 1997).

A broad-spectrum, pan-COV immunogenic composition disclosed herein is administered in an amount sufficient to treat or prevent a Coronavirinae-based infection or pathology. In aspects of this embodiment, the amount of COV antigen and/or immunogenic composition administered is an amount sufficient to reduce one or more physiological conditions or symptom associated with a Coronavirinae-based infection or pathology, an amount sufficient to immunize or vaccinate the individual against the COV infection or pathology, or an amount sufficient to protect the individual against a Coronavirinae-based infection or pathology. As used herein, the term “amount sufficient” includes “effective amount”, “effective dose”, “therapeutically effective amount” or “therapeutically effective dose” and refers to the minimum amount of a COV antigen and/or immunogenic composition necessary to achieve the desired therapeutic effect and includes an amount sufficient to induce or boost an immune response as a function of antigen production in the cell and/or an amount sufficient to reduce or inhibit one or more physiological conditions or symptom associated with a Coronavirinae-based infection or pathology. A prophylactically effective amount is an amount that prevents infection with a Coronavirinae-based infection or pathology at a clinically acceptable level.

In aspects of this embodiment, an effective amount of a broad-spectrum, pan-COV immunogenic composition disclosed herein reduces or inhibits one or more physiological conditions or symptom associated with a Coronavirinae-based infection or pathology by, e.g., at least 10%, at least 20%, at least 30%, at least 40%, at least 50%, at least 60%, at least 70%, at least 80%, at least 90% or at least 100%. In other aspects of this embodiment, an effective amount of a broad-spectrum, pan-COV immunogenic composition disclosed herein reduces or inhibits one or more physiological conditions or symptom associated with a Coronavirinae-based infection or pathology by, e.g., at most 10%, at most 20%, at most 30%, at most 40%, at most 50%, at most 60%, at most 70%, at most 80%, at most 90% or at most 100%. In yet other aspects of this embodiment, an effective amount of a broad-spectrum, pan-COV immunogenic composition disclosed herein reduces or inhibits one or more physiological conditions or symptom associated with a Coronavirinae-based infection or pathology by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In other aspects of this embodiment, an effective amount of a broad-spectrum, pan-COV immunogenic composition disclosed herein reduces or inhibits one or more physiological conditions or symptom associated with a Coronavirinae-based infection or pathology for, e.g., at least one week, at least one month, at least two months, at least three months, at least four months, at least five months, at least six months, at least seven months, at least eight months, at least nine months, at least ten months, at least eleven months, or at least twelve months. In yet other aspects of this embodiment, an effective amount of a broad-spectrum, pan-COV immunogenic composition disclosed herein reduces or inhibits one or more physiological conditions or symptom associated with a Coronavirinae-based infection or pathology for, e.g., about 1 month to about 3 months, about 1 month to about 6 months, about 3 months to about 6 months, about 3 months to about 9 months, about 3 months to about 12 months, about 3 months to about 18 months, about 3 months to about 24 months, about 3 months to about 30 months, about 3 months to about 36 months, about 6 months to about 9 months, about 6 months to about 12 months, about 6 months to about 18 months, about 6 months to about 24 months, about 6 months to about 30 months, about 6 months to about 36 months, about 9 months to about 12 months, about 9 months to about 18 months, about 9 months to about 24 months, about 9 months to about 30 months, about 9 months to about 36 months, about 12 months to about 18 months, about 12 months to about 24 months, about 12 months to about 30 months, about 12 months to about 36 months, about 18 months to about 24 months, about 18 months to about 30 months, about 18 months to about 36 months, about 24 months to about 30 months, about 24 months to about 36 months, about 30 months to about 36 months. In still other aspects of this embodiment, an effective amount of a broad-spectrum, pan-COV immunogenic composition disclosed herein reduces or inhibits one or more physiological conditions or symptom associated with a Coronavirinae-based infection or pathology for, e.g., about 3 years to about 4 years, about 3 years to about 5 years, about 3 years to about 6 years, about 3 years to about 7 years, about 3 years to about 8 years, about 3 years to about 9 years, about 3 years to about 10 years, about 5 years to about 10 years, or about 7 years to about 10 years.

The actual effective amount of a broad-spectrum, pan-COV immunogenic composition disclosed herein to be administered to an individual can be determined by a person of ordinary skill in the art by taking into account factors, including, without limitation, the type of COV infection or pathology, the location of the Coronavirinae-based infection or pathology, the cause of the Coronavirinae-based infection or pathology, the severity of the Coronavirinae-based infection or pathology, the degree of relief desired for a Coronavirinae-based infection or pathology, the duration of relief desired for a Coronavirinae-based infection or pathology, the particular COV antigen and/or broad-spectrum, pan-COV immunogenic composition used, the rate of excretion of the particular COV antigen and/or broad-spectrum, pan-COV immunogenic composition used, the pharmacodynamics of the particular COV antigen and/or broad-spectrum, pan-COV immunogenic composition used, the nature of the other compounds to be included in the broad-spectrum, pan-COV immunogenic composition, the particular route of administration used, the particular means of administration used, the particular target tissue, the particular target cell type, the particular characteristics, history and risk factors of the individual, such as, e.g., age, weight, general health and the like, or any combination thereof. Additionally, where repeated administration of a broad-spectrum, pan-COV immunogenic composition disclosed herein is used, the actual therapeutically effective amount will further depend upon factors, including, without limitation, the frequency of administration, the half-life of a broad-spectrum, pan-COV immunogenic composition disclosed herein, or any combination thereof. It is known by a person of ordinary skill in the art that an effective amount of a broad-spectrum, pan-COV immunogenic composition disclosed herein can be extrapolated from in vitro assays and in vivo administration studies using animal models prior to administration to humans. Wide variations in the necessary effective amount are to be expected in view of the differing efficiencies of the various routes of administration. For instance, oral administration generally would be expected to require higher dosage levels than administration by intravenous or intravitreal injection. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors.

In some embodiments, an effective amount of each COV peptide antigen present in a broad-spectrum, pan-COV immunogenic composition disclosed herein is generally in the range of about 0.001 mg to about 10 mg for each COV peptide antigen. In aspects of these embodiments, an effective amount of each COV peptide antigen present in a broad-spectrum, pan-COV immunogenic composition disclosed herein may be, e.g., at least 0.001 mg, at least 0.005 mg, at least 0.01 mg, at least 0.05 mg, at least 0.1 mg, at least 0.5 mg, at least 1.0 mg, at least 5.0 mg, or at least 10 mg for each COV peptide antigen. In aspects of these embodiments, an effective amount of each COV peptide antigen present in a broad-spectrum, pan-COV immunogenic composition disclosed herein may be, e.g., at most 0.001 mg, at most 0.005 mg, at most 0.01 mg, at most 0.05 mg, at most 0.1 mg, at most 0.5 mg, at most 1.0 mg, at most 5.0 mg, or at most 10 mg for each COV peptide antigen. In yet other aspects of these embodiments, an effective amount of each COV peptide antigen present in a broad-spectrum, pan-COV immunogenic composition disclosed herein may be, e.g., about 0.001 mg to about 0.005 mg, about 0.001 mg to about 0.01 mg, about 0.001 mg to about 0.05 mg, about 0.001 mg to about 0.1 mg, about 0.001 mg to about 0.5 mg, about 0.001 mg to about 1 mg, about 0.001 mg to about 5 mg, about 0.001 mg to about 10 mg, about 0.01 mg to about 0.05 mg, about 0.01 mg to about 0.1 mg, about 0.01 mg to about 0.5 mg, about 0.01 mg to about 1 mg, about 0.01 mg to about 5 mg, about 0.01 mg to about 10 mg, about 0.1 mg to about 0.5 mg, about 0.1 mg to about 1 mg, about 0.1 mg to about 5 mg, about 0.1 mg to about 10 mg, about 1 mg to about 10 mg, or about 5 mg to about 10 mg for each COV peptide antigen.

In some embodiments, an effective amount of a polypeptide including each of the one or more COV peptide antigens disclosed herein present in a broad-spectrum, pan-COV immunogenic composition disclosed herein is generally in the range of about 0.001 mg to about 10 mg. In aspects of these embodiments, an effective amount of a polypeptide including one or more COV peptide antigens disclosed herein present in a broad-spectrum, pan-COV immunogenic composition disclosed herein may be, e.g., at least 0.001 mg, at least 0.005 mg, at least 0.01 mg, at least 0.05 mg, at least 0.1 mg, at least 0.5 mg, at least 1.0 mg, at least 5.0 mg, or at least 10 mg. In aspects of these embodiments, an effective amount of a polypeptide including one or more COV peptide antigens disclosed herein present in a broad-spectrum, pan-COV immunogenic composition disclosed herein may be, e.g., at most 0.001 mg, at most 0.005 mg, at most 0.01 mg, at most 0.05 mg, at most 0.1 mg, at most 0.5 mg, at most 1.0 mg, at most 5.0 mg, or at most 10 mg. In yet other aspects of these embodiments, an effective amount of a polypeptide including one or more COV peptide antigens disclosed herein present in a broad-spectrum, pan-COV immunogenic composition disclosed herein may be, e.g., about 0.001 mg to about 0.005 mg, about 0.001 mg to about 0.01 mg, about 0.001 mg to about 0.05 mg, about 0.001 mg to about 0.1 mg, about 0.001 mg to about 0.5 mg, about 0.001 mg to about 1 mg, about 0.001 mg to about 5 mg, about 0.001 mg to about 10 mg, about 0.01 mg to about 0.05 mg, about 0.01 mg to about 0.1 mg, about 0.01 mg to about 0.5 mg, about 0.01 mg to about 1 mg, about 0.01 mg to about 5 mg, about 0.01 mg to about 10 mg, about 0.1 mg to about 0.5 mg, about 0.1 mg to about 1 mg, about 0.1 mg to about 5 mg, about 0.1 mg to about 10 mg, about 1 mg to about 10 mg, or about 5 mg to about 10 mg.

In some embodiments, an effective amount of each nucleotide sequence encoding one or more COV peptide antigens present in a broad-spectrum, pan-COV immunogenic composition disclosed herein is generally in the range of about 0.1 μg to about 1,000 μg for each nucleotide sequence. In aspects of these embodiments, an effective amount of each nucleotide sequence encoding one or more COV peptide antigens present in a broad-spectrum, pan-COV immunogenic composition disclosed herein may be, e.g., at least 0.1 μg, at least 0.5 μg, at least 1 μg, at least 5.0 μg, at least 10 μg, at least 15 μg, at least 20 μg, at least 25 μg, at least 30 μg, at least 35 μg, at least 40 μg, at least 45 μg, at least 50 μg, at least 60 μg, at least 70 μg, at least 80 μg, at least 90 μg, at least 100 μg, at least 125 μg, at least 150 μg, at least 175 μg, at least 200 μg, at least 225 μg, at least 250 μg, at least 275 μg, at least 300 μg, at least 325 μg, at least 350 μg, at least 375 μg, at least 400 μg, at least 500 μg at least 600 μg at least 700 μg at least 800 μg at least 900 μg, or at least 1,000 μg for each nucleotide sequence.

In aspects of these embodiments, an effective amount of each nucleotide sequence encoding one or more COV peptide antigens present in a broad-spectrum, pan-COV immunogenic composition disclosed herein may be, e.g., about 0.1 μg, about 0.5 μg, about 1 μg to about 5 μg, about 1 μg to about 10 μg, about 1 μg to about 20 μg, about 1 μg to about 30 μg, about 1 μg to about 40 μg, about 1 μg to about 50 μg, about 5 μg to about 10 μg, about 5 μg to about 20 μg, about 5 μg to about 30 μg, about 5 μg to about 40 μg, about 5 μg to about 50 μg, about 5 μg to about 60 μg, about 5 μg to about 70 μg, about 5 μg to about 80 μg, about 5 μg to about 90 μg, about 5 μg to about 100 μg, about 10 μg to about 20 μg, about 10 μg to about 30 μg, about 10 μg to about 40 μg, about 10 μg to about 50 μg, about 10 μg to about 60 μg, about 10 μg to about 70 μg, about 10 μg to about 80 μg, about 10 μg to about 90 μg, about 10 μg to about 100 μg, about 25 μg to about 50 μg, about 25 μg to about 75 μg, about 25 μg to about 100 μg, about 25 μg to about 125 μg, about 25 μg to about 150 μg, about 25 μg to about 175 μg, about 25 μg to about 200 μg, about 25 μg to about 225 μg, about 25 μg to about 250 μg, about 50 μg to about 100 μg, about 50 μg to about 125 μg, about 50 μg to about 150 μg, about 50 μg to about 175 μg, about 50 μg to about 200 μg, about 50 μg to about 225 μg, about 50 μg to about 250 μg, about 100 μg to about 150 μg, about 100 μg to about 200 μg, about 100 μg to about 250 μg, about 100 μg to about 300 μg, about 100 μg to about 350 μg, about 100 μg to about 400 μg, about 150 μg to about 200 μg, about 150 μg to about 250 μg, about 150 μg to about 300 μg, about 150 μg to about 350 μg, about 150 μg to about 400 μg, about 200 μg to about 250 μg, about 200 μg to about 300 μg, about 200 μg to about 350 μg, about 200 μg to about 400 μg, about 250 μg to about 300 μg, about 250 μg to about 350 μg, about 250 μg to about 400 μg, about 300 μg to about 350 μg, about 300 μg to about 400 μg, or about 350 μg to about 400 μg for each nucleotide sequence. In other aspects of these embodiments, an effective amount of each nucleotide sequence encoding one or more COV peptide antigens present in an immunogenic composition disclosed herein may be, e.g., about 300 μg to about 1,000 μg, about 400 μg to about 1,000 μg, about 500 μg to about 1,000 μg, about 600 μg to about 1,000 μg, about 700 μg to about 1,000 μg, about 800 μg to about 1,000 μg, about 900 μg to about 1,000 μg.

In aspects of these embodiments, when a peptide, polypeptide, or a nucleotide sequence is formulated in a viral-based delivery system, such as, e.g., a virus-like particle carrier, an effective amount can be expressed as the amount of a virus-like particle carrier or its multiplicity of infection (MOU) activity. In some embodiments, an effective amount of a viral-based delivery system, such as, e.g., a virus-like particle carrier, can be, e.g., about 1×10¹⁰ viral particles, about 5×10¹⁰ viral particles, about 1×10¹¹ viral particles, about 5×10¹¹ viral particles, or about 1×10¹² virial particles. In some embodiments, an effective amount of a viral-based delivery system, such as, e.g., a virus-like particle carrier, can be, e.g., at least 1×10¹⁰ viral particles, at least 5×10¹⁰ viral particles, at least 1×10¹¹ viral particles, at least 5×10¹¹ viral particles, or at least 1×10¹² virial particles. In some embodiments, an effective amount of a viral-based delivery system, such as, e.g., a virus-like particle carrier, can be, e.g., at most 1×10¹⁰ viral particles, at most 5×10¹⁰ viral particles, at most 1×10¹¹ viral particles, at most 5×10¹¹ viral particles, or at most 1×10¹² virial particles. In some embodiments, an effective amount of a viral-based delivery system, such as, e.g., a virus-like particle carrier, can be, e.g., about 1×10¹⁰ to about 5×10¹⁰ viral particles, about 1×10¹⁰ to about 1×10¹¹ viral particles, about 1×10¹⁰ to about 5×10¹¹ viral particles, about 1×10¹⁰ to about 1×10¹² virial particles, about 5×10¹⁰ to about 1×10¹¹ viral particles, about 5×10¹⁰ to about 5×10¹¹ viral particles, about 5×10¹⁰ to about 1×10¹² virial particles, about 1×10¹¹ to about 5×10¹¹ viral particles, about 1×10¹¹ to about 1×10¹² virial particles, or about 5×10¹¹ to about 1×10¹² virial particles.

Dosing can be single dosage or cumulative (serial dosing), and can be readily determined by one skilled in the art. For instance, treatment of a Coronavirinae-based infection or pathology may comprise a one-time administration of an effective amount of a COV antigen and/or immunogenic composition herein. As a non-limiting example, an effective amount of a COV antigen and/or immunogenic composition disclosed herein can be administered once to an individual, e.g., as a single injection or deposition. Alternatively, treatment of a Coronavirinae-based infection or pathology may comprise multiple administrations of an effective amount of a COV antigen and/or immunogenic composition disclosed herein carried out over a range of time periods, such as, e.g., monthly, once every two months, one every three months, once every four months, one every five months, one every six months, or yearly. As a non-limiting example, a COV antigen and/or immunogenic composition disclosed herein can be administered one, two, three, four, five or six times yearly to an individual. The timing of administration can vary from individual to individual, depending upon such factors as the severity of an individual's symptoms and/or the need for a booster dose. For example, an effective amount of an antigen and/or immunogenic composition disclosed herein can be administered to an individual once every three months for an indefinite period of time, or until the individual no longer requires therapy. A person of ordinary skill in the art will recognize that the condition of the individual can be monitored throughout the course of treatment and that the effective amount of an antigen and/or immunogenic composition disclosed herein that is administered can be adjusted accordingly.

In some embodiments, a COV antigen-specific immune response is characterized by measuring antigenic polypeptide antibody titer from one or a combination of an anti-RdRp from an alphacoronavirus strain, a betacoronavirus strain, a gammacoronavirus strain, and/or a deltacoronavirus strain, and/or an anti-M protein from an alphacoronavirus strain, a betacoronavirus strain, a gammacoronavirus strain, and/or a deltacoronavirus strain produced in a subject administered a broad-spectrum, pan-COV immunogenic composition disclosed herein. An antibody titer is a measurement of the amount of antibodies within a subject, for example, antibodies that are specific to a particular antigen (e.g., one or more of an anti-RdRp from an alphacoronavirus strain, a betacoronavirus strain, a gammacoronavirus strain, and/or a deltacoronavirus strain, and/or an anti-M protein from an alphacoronavirus strain, a betacoronavirus strain, a gammacoronavirus strain, and/or a deltacoronavirus strain) or epitope of an antigen. Antibody titer can be expressed as the inverse of the greatest dilution that provides a positive result. Enzyme-linked immunosorbent assay (ELISA) is a common assay for determining antibody titers, for example. Antibody titer can be expressed as the concentration of antibody when a quantitative ELISA is performed.

In some embodiments, a COV antigen-specific immune response is characterized by measuring T-cell response produced in a subject administered a broad-spectrum, pan-COV immunogenic composition disclosed herein. Non-limiting examples of assays for measuring a T-cell response include limiting dilution culture assays, cytokine ELISA, enzyme-linked immunospot ELISPO) assay, intracellular staining assays, cytokine capture assays, tetramer staining assays, and spectratyping and biosensor assays. Limiting dilution cultures that measure the frequency of T cells that mount a response to a specific antigen by using various dilutions of cells and then measuring the number of wells with no response. Cytokine ELISA that measures cytokines secreted in the supernatant after in vitro stimulation of cells. ELISPOT measures T cell responses by their cytokine production. Intracellular staining assays use fluorescent labels and flow cytometry to measure specific cell types and the cytokines these cells produce. Cytokine capture measures cytokine production using bispecific antibodies that recognize a T cell specific marker and a cytokine of interest to capture and detect the labelled cytokines on the surface of the T cells. Tetramer staining assays use fluorescent labels and flow cytometry to measure specific cell types by their receptors.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by a broad-spectrum, pan-COV immunogenic composition disclosed herein.

A COV antigen and/or immunogenic composition disclosed herein can also be administered to an individual in combination with other therapeutic/prophylactic compounds to increase the overall therapeutic effect of the treatment. The use of multiple compounds to treat an indication can increase the beneficial effects while reducing the presence of side effects. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. In some embodiments, the time of administration between the initial administration of the prophylactic composition and the booster may be, but is not limited to, 1 week, 2 weeks, 3 weeks, 1 month, 2 months, 3 months, 6 months, 1 year, 2 years, 3 years, 4 years, 5 years, 6 years, 7 years, 8 years, 9 years, or 10 years.

Aspects of the present specification can also be described by the following embodiments:
1. A broad-spectrum, pan-COV immunogenic composition comprising a nucleotide sequence comprising an open reading frame encoding a polypeptide comprising 2 or more COV peptide antigens, in any combination or order thereof, wherein each of the 2 or more different COV peptide antigens are obtained from a RNA dependent RNA polymerase from an alphacoronavirus species, strain, genotype, subgenotype, serotypes, or genetic/antigenic lineage, a RNA dependent RNA polymerase from a betacoronavirus species, strain, genotype, subgenotype, serotypes, or genetic/antigenic lineage, a RNA dependent RNA polymerase from a gammacoronavirus species, strain, genotype, subgenotype, serotypes, or genetic/antigenic lineage, a RNA dependent RNA polymerase from a deltacoronavirus species, strain, genotype, subgenotype, serotypes, or genetic/antigenic lineage, a M protein from an alphacoronavirus species, strain, genotype, subgenotype, serotypes, or genetic/antigenic lineage, a M protein from a betacoronavirus species, strain, genotype, subgenotype, serotypes, or genetic/antigenic lineage, a M protein from a gammacoronavirus species, strain, genotype, subgenotype, serotypes, or genetic/antigenic lineage, and/or a M protein from a deltacoronavirus species, strain, genotype, subgenotype, serotypes, or genetic/antigenic lineage.
2. The broad-spectrum, pan-COV immunogenic composition of embodiment 1, wherein the 2 or more COV peptide antigens comprise at least one of the COV peptide antigens being from a RNA dependent RNA polymerase from an alphacoronavirus species, strain, genotype, subgenotype, serotypes, or genetic/antigenic lineage, at least one of the COV peptide antigens being from a RNA dependent RNA polymerase from a betacoronavirus species, strain, genotype, subgenotype, serotypes, or genetic/antigenic lineage, at least one of the COV peptide antigens being from a RNA dependent RNA polymerase from a gammacoronavirus species, strain, genotype, subgenotype, serotypes, or genetic/antigenic lineage, at least one of the COV peptide antigens being from a RNA dependent RNA polymerase from a deltacoronavirus species, strain, genotype, subgenotype, serotypes, or genetic/antigenic lineage, at least one of the COV peptide antigens being from a M protein from an alphacoronavirus species, strain, genotype, subgenotype, serotypes, or genetic/antigenic lineage, at least one of the COV peptide antigens being from a M protein from a betacoronavirus species, strain, genotype, subgenotype, serotypes, or genetic/antigenic lineage, at least one of the COV peptide antigens being from a M protein from a gammacoronavirus species, strain, genotype, subgenotype, serotypes, or genetic/antigenic lineage, and/or at least one of the COV peptide antigens being from a M protein from a deltacoronavirus species, strain, genotype, subgenotype, serotypes, or genetic/antigenic lineage.
3. A broad-spectrum, pan-COV immunogenic composition comprising a nucleotide sequence comprising an open reading frame encoding a polypeptide comprising 2 or more COV peptide antigens, in any combination or order thereof, wherein each of the 2 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
4. The broad-spectrum, pan-COV immunogenic composition of embodiment 3, wherein each of the 2 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
5. The broad-spectrum, pan-COV immunogenic composition of any one of embodiments 1-4, wherein a peptide linker/spacer separates a subset of the two or more different COV peptide antigens.
6. The broad-spectrum, pan-COV immunogenic composition of any one of embodiments 1-4, wherein a peptide linker/spacer separates each of the two or more different COV peptide antigens.
7. The broad-spectrum, pan-COV immunogenic composition of any one of embodiments 5 or 6, wherein each of the peptide linker/spacers has the amino acid sequence of SEQ ID NO: 18, SEQ ID NO: 19, SEQ ID NO: 20, or SEQ ID NO: 21.
8. The broad-spectrum, pan-COV immunogenic composition of any one of embodiments 1-7, wherein the 2 or more COV peptide antigens is 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more COV peptide antigens, in any combination or order thereof, or wherein the 2 or more COV peptide antigens is from 2 to 48, 4 to 46, 6 to 44, 8 to 42, 10 to 40, 12 to 38, 14 to 36, 16 to 34, 18 to 32, or 20 to 30, in any combination or order thereof, or wherein the 2 or more COV peptide antigens is any integer from 0 to 10, 0 to 20, 0 to 30, 0 to 40, 0 to 50, 5 to 10, 5 to 20, 5 to 30, 5 to 40, 5 to 50, 10 to 20, 10 to 30, 10 to 40, 10 to 50, in any combination or order thereof, or wherein the 2 or more COV peptide antigens is is any integer from 0 to 4, 4, to 8, 8 to 12, 12 to 16, 16 to 20, 20 to 24, 24 to 28, 28 to 32, 32 to 36, 36 to 40, 40 to 44, 44 to 48, or 48 to 52, in any combination or order thereof.
9. A broad-spectrum, pan-COV immunogenic composition comprising a nucleotide sequence comprising an open reading frame encoding a polypeptide of Polypeptide Structure I:

COVPA¹-(PLS¹ₘ[COVPA²-(PLS²ₘ)]ₙ-COVPA³ Polypeptide Structure I

wherein COVPA¹ is a first COV peptide antigen selected from a COV peptide antigen having an amino acid sequence of at least 75% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of at least 75% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of at least 75% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer, m is independently any integer from 0 to 1, n is any integer from 0 to 50, wherein the polypeptide comprises at least two peptide antigens selected from COVPA1, COVPA2, COVPA3, or any combination thereof, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other.
10. The broad-spectrum, pan-COV immunogenic composition of embodiment 9, wherein COVPA¹, COVPA², and COVPA³ each have a different peptide antigen from each other.
11. The broad-spectrum, pan-COV immunogenic composition of embodiment 9 or 10, wherein COVPA¹ is a first COV peptide antigen selected from a COV peptide antigen having an amino acid sequence of at least 85% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
12. The broad-spectrum, pan-COV immunogenic composition of any one of embodiments 9-11, wherein COVPA¹ is a first COV peptide antigen selected from a COV peptide antigen having an amino acid sequence of at least 95% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
13. The broad-spectrum, pan-COV immunogenic composition of any one of embodiments 9-12, wherein COVPA¹ is a first COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
14. The broad-spectrum, pan-COV immunogenic composition of any one of embodiments 9-13, wherein COVPA² is a second COV peptide antigen selected from a COV peptide antigen having an amino acid sequence of at least 85% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
15. The broad-spectrum, pan-COV immunogenic composition of any one of embodiments 9-14, wherein COVPA² is a second COV peptide antigen selected from a COV peptide antigen having an amino acid sequence of at least 95% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
16. The broad-spectrum, pan-COV immunogenic composition of any one of embodiments 9-15, wherein COVPA² is a second COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
17. The broad-spectrum, pan-COV immunogenic composition of any one of embodiments 9-16, wherein COVPA³ is a third COV peptide antigen selected from a COV peptide antigen having an amino acid sequence of at least 85% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
18. The broad-spectrum, pan-COV immunogenic composition of any one of embodiments 9-17, wherein COVPA³ is a third COV peptide antigen selected from a COV peptide antigen having an amino acid sequence of at least 95% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
19. The broad-spectrum, pan-COV immunogenic composition of any one of embodiments 9-18, wherein COVPA³ is a third COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
20. The broad-spectrum, pan-COV immunogenic composition of any one of embodiments 9-19, wherein PLS¹ has an amino acid sequence of SEQ ID NO: 18, SEQ ID NO: 19, SEQ ID NO: 20, or SEQ ID NO: 21.
21. The broad-spectrum, pan-COV immunogenic composition of any one of embodiments 9-20, wherein PLS² has an amino acid sequence of SEQ ID NO: 18, SEQ ID NO: 19, SEQ ID NO: 20, or SEQ ID NO: 21.
22. The broad-spectrum, pan-COV immunogenic composition of any one of embodiments 9-21, wherein n is any integer from 2 to 48, 4 to 46, 6 to 44, 8 to 42, 10 to 40, 12 to 38, 14 to 36, 16 to 34, 18 to 32, or 20 to 30.
23. The broad-spectrum, pan-COV immunogenic composition of any one of embodiments 9-21, wherein n is any integer from 0 to 10, 0 to 20, 0 to 30, 0 to 40, 0 to 50, 5 to 10, 5 to 20, 5 to 30, 5 to 40, 5 to 50, 10 to 20, 10 to 30, 10 to 40, 10 to 50.
24. The broad-spectrum, pan-COV immunogenic composition of any one of embodiments 19-21, wherein n is any integer from 0 to 4, 4, to 8, 8 to 12, 12 to 16, 16 to 20, 20 to 24, 24 to 28, 28 to 32, 32 to 36, 36 to 40, 40 to 44, 44 to 48, or 48 to 52.
25. The broad-spectrum, pan-COV immunogenic composition of any one of embodiments 1-24, wherein the open reading frame further encodes a signal peptide operably linked to and in frame with the open reading frame encoding the polypeptide.
26. The broad-spectrum, pan-COV immunogenic composition of embodiments 25, wherein the signal peptide is present at the amino-terminus of the polypeptide or the carboxy-terminus of the polypeptide.
27. The broad-spectrum, pan-COV immunogenic composition of any one of embodiments 1-26, wherein the nucleotide sequence further comprises an open reading frame encoding an adjuvant operably linked to and in frame with the open reading frame encoding the polypeptide.
28. The broad-spectrum, pan-COV immunogenic composition of embodiment 27, wherein the adjuvant is present at the amino-terminus of the polypeptide or the carboxy-terminus of the polypeptide.
29. The broad-spectrum, pan-COV immunogenic composition of any one of embodiments 1-28, wherein the nucleotide sequence further comprises an open reading frame for a stabilizing peptide operably linked to and in frame with the open reading frame encoding the polypeptide.
30. The broad-spectrum, pan-COV immunogenic composition of embodiment 29, wherein the stabilizing peptide is operably linked to the amino-terminus of the polypeptide or the carboxy-terminus of the polypeptide.
31. The broad-spectrum, pan-COV immunogenic composition of embodiment 29, wherein the polypeptide is inserted within the stabilizing peptide.
32. The broad-spectrum, pan-COV immunogenic composition of any one of embodiments 29-31, wherein the stabilizing peptide is a lysosomal-associated membrane protein (LAMP) or fragment thereof.
33. The broad-spectrum, pan-COV immunogenic composition of embodiment 32, wherein the LAMP fragment comprises one or more of a first luminal domain, a second luminal domain, a hinge domain, a transmembrane domain, portions thereof, or any combination thereof.
34. The broad-spectrum, pan-COV immunogenic composition of embodiment 32 or 33, wherein the LAMP or fragment thereof is a LAMP-1, a LAMP-2, or a LAMP-3.
35. The broad-spectrum, pan-COV immunogenic composition of any one of embodiments 1-34, wherein the nucleotide-sequence is a DNA nucleotide sequence, a RNA nucleotide sequence, or a self-amplifying RNA nucleotide sequence.
36. The broad-spectrum, pan-COV immunogenic composition of embodiment 35, wherein the RNA nucleotide sequence is a mRNA nucleotide sequence.
37. The broad-spectrum, pan-COV immunogenic composition of embodiment 36, wherein the mRNA nucleotide sequence comprises one or more ribonucleic acid analogs or derivatives.
38. The broad-spectrum, pan-COV immunogenic composition of embodiment 37, wherein the mRNA nucleotide sequence comprises one or more ribonucleotide analogs or derivatives, one or more ribonucleoside analogs or derivatives, or both.
39. The broad-spectrum, pan-COV immunogenic composition of embodiment 38, wherein the one or more ribonucleotide analogs or derivatives include a bridged nucleic acid (BNA), a cyclohexenyl nucleic acid (CeNA), an ethylene nucleic acid (ENA), a 2′-fluoro-hexitol nucleic acid (FHNA), a glycol nucleic acid (GNA), a locked nucleic acid (LNA), including a LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), a peptide nucleic acid (PNA), a threose nucleic acid (TNA), or any combination thereof.
40. The broad-spectrum, pan-COV immunogenic composition of embodiments 38 or 39, wherein the one or more ribonucleoside analogs or derivatives include a 2′-O-methyl modified nucleoside, a nucleoside comprising a 5′ phosphorothioate group, 5′ phosphate group, 5′ triphosate group, 5′ phosphorodithioate group, a terminal nucleoside linked to a cholesteryl derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, 2′-alkoxyalkyl-modified nucleoside e.g., (2′-O-methoxyethyl) nucleoside, a phosphorodiamidate nucleoside, a phosphoramidate (amidophosphate) nucleoside, a morpholino nucleoside, a non-natural base comprising ribonucleoside, or any combination thereof.
41. The broad-spectrum, pan-COV immunogenic composition of any one of embodiments 1-40, wherein the nucleotide sequence is codon optimized for translation in a human.
42. A broad-spectrum, pan-COV immunogenic composition comprising two or more nucleotide sequences, the two or more nucleotide sequences each comprising an open reading frame encoding a polypeptide comprising 2 or more COV peptide antigens, in any combination or order thereof, wherein each of the 2 or more different COV peptide antigens are obtained from a RNA dependent RNA polymerase from an alphacoronavirus species, strain, genotype, subgenotype, serotypes, or genetic/antigenic lineage, a RNA dependent RNA polymerase from a betacoronavirus species, strain, genotype, subgenotype, serotypes, or genetic/antigenic lineage, a RNA dependent RNA polymerase from a gammacoronavirus species, strain, genotype, subgenotype, serotypes, or genetic/antigenic lineage, a RNA dependent RNA polymerase from a deltacoronavirus species, strain, genotype, subgenotype, serotypes, or genetic/antigenic lineage, a M protein from an alphacoronavirus species, strain, genotype, subgenotype, serotypes, or genetic/antigenic lineage, a M protein from a betacoronavirus species, strain, genotype, subgenotype, serotypes, or genetic/antigenic lineage, a M protein from a gammacoronavirus species, strain, genotype, subgenotype, serotypes, or genetic/antigenic lineage, and/or a M protein from a deltacoronavirus species, strain, genotype, subgenotype, serotypes, or genetic/antigenic lineage.
43. The broad-spectrum, pan-COV immunogenic composition of embodiment 42, wherein the 2 or more COV peptide antigens comprise at least one of the COV peptide antigens being from a RNA dependent RNA polymerase from an alphacoronavirus species, strain, genotype, subgenotype, serotypes, or genetic/antigenic lineage, at least one of the COV peptide antigens being from a RNA dependent RNA polymerase from a betacoronavirus species, strain, genotype, subgenotype, serotypes, or genetic/antigenic lineage, at least one of the COV peptide antigens being from a RNA dependent RNA polymerase from a gammacoronavirus species, strain, genotype, subgenotype, serotypes, or genetic/antigenic lineage, at least one of the COV peptide antigens being from a RNA dependent RNA polymerase from a deltacoronavirus species, strain, genotype, subgenotype, serotypes, or genetic/antigenic lineage, at least one of the COV peptide antigens being from a M protein from an alphacoronavirus species, strain, genotype, subgenotype, serotypes, or genetic/antigenic lineage, at least one of the COV peptide antigens being from a M protein from a betacoronavirus species, strain, genotype, subgenotype, serotypes, or genetic/antigenic lineage, at least one of the COV peptide antigens being from a M protein from a gammacoronavirus species, strain, genotype, subgenotype, serotypes, or genetic/antigenic lineage, and/or at least one of the COV peptide antigens being from a M protein from a deltacoronavirus species, strain, genotype, subgenotype, serotypes, or genetic/antigenic lineage.
44. A broad-spectrum, pan-COV immunogenic composition comprising two or more nucleotide sequences, the two or more nucleotide sequences each comprising an open reading frame encoding a polypeptide comprising 2 or more COV peptide antigens, in any combination or order thereof, wherein each of the 2 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
45. The broad-spectrum, pan-COV immunogenic composition of embodiment 44, wherein each of the 2 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
46. The broad-spectrum, pan-COV immunogenic composition of any one of embodiments 42-45, wherein a peptide linker/spacer separates a subset of the two or more different COV peptide antigens.
47. The broad-spectrum, pan-COV immunogenic composition of any one of embodiments 42-45, wherein a peptide linker/spacer separates each of the two or more different COV peptide antigens.
48. The broad-spectrum, pan-COV immunogenic composition of any one of embodiments 46 or 47, wherein each of the peptide linker/spacers has the amino acid sequence of SEQ ID NO: 18, SEQ ID NO: 19, SEQ ID NO: 20, or SEQ ID NO: 21.
49. The broad-spectrum, pan-COV immunogenic composition of any one of embodiments 42-48, wherein the two or more nucleotide sequences is 3 or more, 4 or more, 5 or more, 6 or more, 7 or more, 8 or more, 9 or more, or 10 or more nucleotide sequences.
50. The broad-spectrum, pan-COV immunogenic composition of any one of embodiments 42-49, wherein the 2 or more COV peptide antigens is 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more COV peptide antigens, in any combination or order thereof, or wherein the 2 or more COV peptide antigens is from 2 to 60, 4 to 56, 6 to 54, 8 to 52, 10 to 50, 12 to 48, 14 to 46, 16 to 44, 18 to 42, or 20 to 40, in any combination or order thereof, or wherein the 2 or more COV peptide antigens is is any integer from 0 to 10, 0 to 20, 0 to 30, 0 to 40, 0 to 50, 5 to 10, 5 to 20, 5 to 30, 5 to 40, 5 to 50, 10 to 20, 10 to 30, 10 to 40, 10 to 50, in any combination or order thereof, or wherein the 2 or more COV peptide antigens is is any integer from 0 to 4, 4, to 8, 8 to 12, 12 to 16, 16 to 20, 20 to 24, 24 to 28, 28 to 32, 32 to 36, 36 to 40, 40 to 44, 44 to 48, or 48 to 52, in any combination or order thereof.
51. The broad-spectrum, pan-COV immunogenic composition of any one of embodiments 42-50, wherein each of the two or more nucleotide sequences further encodes a signal peptide operably linked to and in frame with the open reading frame encoding the polypeptide.
52. The broad-spectrum, pan-COV immunogenic composition of embodiments 51, wherein the signal peptide is present at the amino-terminus of the polypeptide or the carboxy-terminus of the polypeptide.
53. The broad-spectrum, pan-COV immunogenic composition of any one of embodiments 42-52, wherein each the two or more nucleotide sequences further comprises an open reading frame encoding an adjuvant operably linked to and in frame with the open reading frame encoding the polypeptide.
54. The broad-spectrum, pan-COV immunogenic composition of embodiment 53, wherein the adjuvant is present at the amino-terminus of the polypeptide or the carboxy-terminus of the polypeptide.
55. The broad-spectrum, pan-COV immunogenic composition of any one of embodiments 42-54, wherein each the two or more nucleotide sequences further comprises an open reading frame for a stabilizing peptide operably linked to and in frame with the open reading frame encoding the polypeptide.
56. The broad-spectrum, pan-COV immunogenic composition of embodiment 55, wherein the stabilizing peptide is operably linked to the amino-terminus of the polypeptide or the carboxy-terminus of the polypeptide.
57. The broad-spectrum, pan-COV immunogenic composition of embodiment 56, wherein the polypeptide is inserted within the stabilizing peptide.
58. The broad-spectrum, pan-COV immunogenic composition of any one of embodiments 55-57, wherein the stabilizing peptide is a lysosomal-associated membrane protein (LAMP) or fragment thereof.
59. The broad-spectrum, pan-COV immunogenic composition of embodiment 58, wherein the LAMP fragment comprises one or more of a first luminal domain, a second luminal domain, a hinge domain, a transmembrane domain, portions thereof, or any combination thereof.
60. The broad-spectrum, pan-COV immunogenic composition of embodiment 58 or 59, wherein the LAMP or fragment thereof is a LAMP-1, a LAMP-2, or a LAMP-3.
61. The broad-spectrum, pan-COV immunogenic composition of any one of embodiments 42-60, wherein each of the two or more nucleotide sequences is a DNA nucleotide sequence, a RNA nucleotide sequence, or a self-amplifying RNA nucleotide sequence.
62. The broad-spectrum, pan-COV immunogenic composition of embodiment 61, wherein the RNA nucleotide sequence is a mRNA nucleotide sequence.
63. The broad-spectrum, pan-COV immunogenic composition of embodiment 62, wherein the mRNA nucleotide sequence comprises one or more ribonucleic acid analogs or derivatives.
64. The broad-spectrum, pan-COV immunogenic composition of embodiment 63, wherein the mRNA nucleotide sequence comprises one or more ribonucleotide analogs or derivatives, one or more ribonucleoside analogs or derivatives, or both.
65. The broad-spectrum, pan-COV immunogenic composition of embodiment 64, wherein the one or more ribonucleotide analogs or derivatives include a bridged nucleic acid (BNA), a cyclohexenyl nucleic acid (CeNA), an ethylene nucleic acid (ENA), a 2′-fluoro-hexitol nucleic acid (FHNA), a glycol nucleic acid (GNA), a locked nucleic acid (LNA), including a LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), a peptide nucleic acid (PNA), a threose nucleic acid (TNA), or any combination thereof.
66. The broad-spectrum, pan-COV immunogenic composition of embodiments 64 or 65, wherein the one or more ribonucleoside analogs or derivatives include a 2′-O-methyl modified nucleoside, a nucleoside comprising a 5′ phosphorothioate group, 5′ phosphate group, 5′ triphosate group, 5′ phosphorodithioate group, a terminal nucleoside linked to a cholesteryl derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, 2′-alkoxyalkyl-modified nucleoside e.g., (2′-O-methoxyethyl) nucleoside, a phosphorodiamidate nucleoside, a phosphoramidate (amidophosphate) nucleoside, a morpholino nucleoside, a non-natural base comprising ribonucleoside, or any combination thereof.
67. The broad-spectrum, pan-COV immunogenic composition of any one of embodiments 42-66, wherein each of the two or more nucleotide sequences is codon optimized for translation in a human.
68. A broad-spectrum, pan-COV immunogenic composition comprising a nucleotide sequence comprising an open reading frame encoding a polypeptide of Polypeptide Structure IV:

PA¹-(SCP¹)[PA²-(SCP²)]ₒ-PA³ Polypeptide Structure IV
wherein PA¹, PA², and PA³ each independently comprise the polypeptide according to any one of embodiments 1-67, wherein SCP¹ and SCP² are each independently a self-cleaving peptide, and wherein o from Polypeptide Structure IV is any integer from 0 to 10.
69. The broad-spectrum, pan-COV immunogenic composition of embodiment 68, wherein PA¹, PA², and PA³ can each independently encode the same polypeptide or a different polypeptide from each other.
70. The broad-spectrum, pan-COV immunogenic composition of embodiment 68 or 69, wherein an internal ribosome entry site is independently operationally linked to the 5′ end of the nucleotide sequence encoding each PA², to the 5′ end of the nucleotide sequence encoding PA³, or to any combination thereof.
71. A broad-spectrum, pan-COV immunogenic composition comprising a polypeptide comprising 2 or more COV peptide antigens, in any combination or order thereof, wherein each of the 2 or more different COV peptide antigens are obtained from a RNA dependent RNA polymerase from an alphacoronavirus species, strain, genotype, subgenotype, serotypes, or genetic/antigenic lineage, a RNA dependent RNA polymerase from a betacoronavirus species, strain, genotype, subgenotype, serotypes, or genetic/antigenic lineage, a RNA dependent RNA polymerase from a gammacoronavirus species, strain, genotype, subgenotype, serotypes, or genetic/antigenic lineage, a RNA dependent RNA polymerase from a deltacoronavirus species, strain, genotype, subgenotype, serotypes, or genetic/antigenic lineage, a M protein from an alphacoronavirus species, strain, genotype, subgenotype, serotypes, or genetic/antigenic lineage, a M protein from a betacoronavirus species, strain, genotype, subgenotype, serotypes, or genetic/antigenic lineage, a M protein from a gammacoronavirus species, strain, genotype, subgenotype, serotypes, or genetic/antigenic lineage, and/or a M protein from a deltacoronavirus species, strain, genotype, subgenotype, serotypes, or genetic/antigenic lineage, in any combination or order thereof.
72. The broad-spectrum, pan-COV immunogenic composition of embodiment 71, wherein the 2 or more COV peptide antigens comprise at least one of the COV peptide antigens being from a RNA dependent RNA polymerase from an alphacoronavirus species, strain, genotype, subgenotype, serotypes, or genetic/antigenic lineage, at least one of the COV peptide antigens being from a RNA dependent RNA polymerase from a betacoronavirus species, strain, genotype, subgenotype, serotypes, or genetic/antigenic lineage, at least one of the COV peptide antigens being from a RNA dependent RNA polymerase from a gammacoronavirus species, strain, genotype, subgenotype, serotypes, or genetic/antigenic lineage, at least one of the COV peptide antigens being from a RNA dependent RNA polymerase from a deltacoronavirus species, strain, genotype, subgenotype, serotypes, or genetic/antigenic lineage, at least one of the COV peptide antigens being from a M protein from an alphacoronavirus species, strain, genotype, subgenotype, serotypes, or genetic/antigenic lineage, at least one of the COV peptide antigens being from a M protein from a betacoronavirus species, strain, genotype, subgenotype, serotypes, or genetic/antigenic lineage, at least one of the COV peptide antigens being from a M protein from a gammacoronavirus species, strain, genotype, subgenotype, serotypes, or genetic/antigenic lineage, and/or at least one of the COV peptide antigens being from a M protein from a deltacoronavirus species, strain, genotype, subgenotype, serotypes, or genetic/antigenic lineage.
73. A broad-spectrum, pan-COV immunogenic composition comprising a polypeptide comprising 2 or more COV peptide antigens, in any combination or order thereof, wherein each of the 2 or more different COV peptide antigens has an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
74. The broad-spectrum, pan-COV immunogenic composition of embodiment 73, wherein each of the 2 or more different COV peptide antigens has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
75. The broad-spectrum, pan-COV immunogenic composition of any one of embodiments 71-74, wherein a peptide linker/spacer separates a subset of the two or more different COV peptide antigens.
76. The broad-spectrum, pan-COV immunogenic composition of any one of embodiments 71-74, wherein a peptide linker/spacer separates each of the two or more different COV peptide antigens.
77. The broad-spectrum, pan-COV immunogenic composition of any one of embodiments 75 or 76, wherein each of the peptide linker/spacers has the amino acid sequence of SEQ ID NO: 18, SEQ ID NO: 19, SEQ ID NO: 20, or SEQ ID NO: 21.
78. The broad-spectrum, pan-COV immunogenic composition of any one of embodiments 71-77, wherein the 2 or more COV peptide antigens is 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 26 or more, 28 or more, 30 or more, 32 or more, 34 or more, 36 or more, 38 or more, 40 or more, 42 or more, 44 or more, 46 or more, 48 or more, or 50 or more COV peptide antigens, in any combination or order thereof, or wherein the 2 or more COV peptide antigens is from 2 to 60, 4 to 56, 6 to 54, 8 to 52, 10 to 50, 12 to 48, 14 to 46, 16 to 44, 18 to 42, or 20 to 40, in any combination or order thereof, or wherein the 2 or more COV peptide antigens is is any integer from 0 to 10, 0 to 20, 0 to 30, 0 to 40, 0 to 50, 5 to 10, 5 to 20, 5 to 30, 5 to 40, 5 to 50, 10 to 20, 10 to 30, 10 to 40, 10 to 50, in any combination or order thereof, or wherein the 2 or more COV peptide antigens is is any integer from 0 to 4, 4, to 8, 8 to 12, 12 to 16, 16 to 20, 20 to 24, 24 to 28, 28 to 32, 32 to 36, 36 to 40, 40 to 44, 44 to 48, or 48 to 52, in any combination or order thereof.
79. A broad-spectrum, pan-COV immunogenic composition comprising a polypeptide of Polypeptide Structure I:

COVPA¹-(PLS¹ₘ)-[COVPA²-(PLS²ₘ)]ₙ-COVPA³ Polypeptide Structure I
wherein COVPA¹ is a first COV peptide antigen selected from a COV peptide antigen having an amino acid sequence of at least 75% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA² is a second COV peptide antigen selected from a COV peptide antigen having an amino acid sequence of at least 75% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, COVPA³ is a third COV peptide antigen selected from a COV peptide antigen having an amino acid sequence of at least 75% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, PLS¹ and PLS² are each independently a peptide linker/spacer, m is independently any integer from 0 to 1, n is any integer from 0 to 52, wherein the polypeptide comprises at least two peptide antigens selected from COVPA1, COVPA2, and COVPA3, or any combination thereof, wherein for each n, COVPA² can be a different peptide antigen or the same peptide antigen, and wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other.
80. The broad-spectrum, pan-COV immunogenic composition of embodiment 79, wherein COVPA¹, COVPA², and COVPA³ each have a different peptide antigen from each other.
81. The broad-spectrum, pan-COV immunogenic composition of embodiment 79 or 80, wherein COVPA¹ is a first COV peptide antigen selected from a COV peptide antigen having an amino acid sequence of at least 85% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
82. The broad-spectrum, pan-COV immunogenic composition of any one of embodiments 79-81, wherein COVPA¹ is a first COV peptide antigen selected from a COV peptide antigen having an amino acid sequence of at least 95% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
83. The broad-spectrum, pan-COV immunogenic composition of any one of embodiments 79-82, wherein COVPA¹ is a first COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
84. The broad-spectrum, pan-COV immunogenic composition of any one of embodiments 79-83, wherein COVPA² is a second COV peptide antigen selected from a COV peptide antigen having an amino acid sequence of at least 85% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
85. The broad-spectrum, pan-COV immunogenic composition of any one of embodiments 79-84, wherein COVPA² is a second COV peptide antigen selected from a COV peptide antigen having an amino acid sequence of at least 95% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
86. The broad-spectrum, pan-COV immunogenic composition of any one of embodiments 79-85, wherein COVPA² is a second COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
87. The broad-spectrum, pan-COV immunogenic composition of any one of embodiments 79-86, wherein COVPA³ is a third COV peptide antigen selected from a COV peptide antigen having an amino acid sequence of at least 85% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
88. The broad-spectrum, pan-COV immunogenic composition of any one of embodiments 79-87, wherein COVPA³ is a third COV peptide antigen selected from a COV peptide antigen having an amino acid sequence of at least 95% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
89. The broad-spectrum, pan-COV immunogenic composition of any one of embodiments 79-88, wherein COVPA³ is a third COV peptide antigen selected from a COV peptide antigen having an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
90. The broad-spectrum, pan-COV immunogenic composition of any one of embodiments 79-89, wherein PLS¹ has an amino acid sequence of SEQ ID NO: 18, SEQ ID NO: 19, SEQ ID NO: 20, or SEQ ID NO: 21.
91. The broad-spectrum, pan-COV immunogenic composition of any one of embodiments 79-90, wherein PLS² has an amino acid sequence of SEQ ID NO: 18, SEQ ID NO: 19, SEQ ID NO: 20, or SEQ ID NO: 21.
92. The broad-spectrum, pan-COV immunogenic composition of any one of embodiments 79-91, wherein n is any integer from 2 to 60, 4 to 56, 6 to 54, 8 to 52, 10 to 50, 12 to 48, 14 to 46, 16 to 44, 18 to 42, or 20 to 40.
93. The broad-spectrum, pan-COV immunogenic composition of any one of embodiments 79-92, wherein n is any integer from 0 to 10, 0 to 20, 0 to 30, 0 to 40, 0 to 50, 5 to 10, 5 to 20, 5 to 30, 5 to 40, 5 to 50, 10 to 20, 10 to 30, 10 to 40, 10 to 50.
94. The broad-spectrum, pan-COV immunogenic composition of any one of embodiments 79-93, wherein n is any integer from 0 to 4, 4, to 8, 8 to 12, 12 to 16, 16 to 20, 20 to 24, 24 to 28, 28 to 32, 32 to 36, 36 to 40, 40 to 44, 44 to 48, or 48 to 52.
95. The broad-spectrum, pan-COV immunogenic composition of any one of embodiments 71-94, wherein a signal peptide is operably linked the polypeptide.
96. The broad-spectrum, pan-COV immunogenic composition of embodiments 95, wherein the signal peptide is present at the amino-terminus of the polypeptide or the carboxy-terminus of the polypeptide.
97. The broad-spectrum, pan-COV immunogenic composition of any one of embodiments 71-96, wherein an adjuvant operably linked to the polypeptide.
98. The broad-spectrum, pan-COV immunogenic composition of embodiment 97, wherein the adjuvant is present at the amino-terminus of the polypeptide or the carboxy-terminus of the polypeptide.
99. A broad-spectrum, pan-COV immunogenic composition comprising a polypeptide of Polypeptide Structure IV:

PA¹-(SCP¹)[PA²-(SCP²)]ₒ-PA³ Polypeptide Structure IV

wherein PA¹, PA², and PA³ each independently comprise the polypeptide according to any one of embodiments 71-98, wherein SOP¹ and SCP² are each independently a self-cleaving peptide, and wherein o from Polypeptide Structure IV is any integer from 0 to 3.
100. The broad-spectrum, pan-COV immunogenic composition of embodiment 99, wherein PA¹, PA², and PA³ can each independently encode the same polypeptide or a different polypeptide from each other.
101. The broad-spectrum, pan-COV immunogenic composition of embodiment 99 or 100, wherein an internal ribosome entry site is independently operationally linked to the 5′ end of the nucleotide sequence encoding each PA², to the 5′ end of the nucleotide sequence encoding PA³, or to any combination thereof.
102. The broad-spectrum, pan-COV immunogenic composition of any one of embodiments 71-101, wherein the polypeptide is recombinantly produced.
103. A broad-spectrum, pan-COV immunogenic composition comprising one or more COV peptide antigens, the one or more COV peptide antigens having an amino acid sequence of at least 75% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
104. The broad-spectrum, pan-COV immunogenic composition of embodiment 103, wherein the one or more COV peptide antigens have an amino acid sequence of at least 85% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
105. The broad-spectrum, pan-COV immunogenic composition of embodiment 103 or 104, wherein the one or more COV peptide antigens have an amino acid sequence of at least 95% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
106. The broad-spectrum, pan-COV immunogenic composition of any one of embodiments 103-105, wherein the one or more COV peptide antigens have the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.
107. The broad-spectrum, pan-COV immunogenic composition of any one of embodiments 103-106, wherein the one or more COV peptide antigens include 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more COV peptide antigens.
108. The broad-spectrum, pan-COV immunogenic composition of any one of embodiments 103-107, wherein the one or more COV peptide antigens a synthetically-produced peptide, a recombinantly-produced peptide, or a peptidomimetic.
109. The broad-spectrum, pan-COV immunogenic composition of any one of embodiments 1-70 or the broad-spectrum, pan-COV immunogenic composition of any one of embodiments 71-108, further comprising one or more adjuvants.
110. The broad-spectrum, pan-COV immunogenic composition of embodiment 109 or the broad-spectrum, pan-COV immunogenic composition of embodiment 109, wherein the one or more adjuvants are encoded by a separate nucleotide sequence.
111. The broad-spectrum, pan-COV immunogenic composition of embodiment 109 or the broad-spectrum, pan-COV immunogenic composition of embodiment 109, wherein the one or more adjuvants comprise one or more organic adjuvants, one or more inorganic adjuvants, or any combination thereof.
112. The broad-spectrum, pan-COV immunogenic composition of embodiment 111 or the broad-spectrum, pan-COV immunogenic composition of embodiment 111, wherein the one or more organic adjuvants comprise an emulsion adjuvant, a pathogen associated molecular pattern (PAMP) adjuvant, a molecular adjuvant, a poly (acrylic acid) adjuvant, a polyphosphazene (PZ) adjuvant, or any combination thereof.
113. The broad-spectrum, pan-COV immunogenic composition of embodiment 112 or the broad-spectrum, pan-COV immunogenic composition of embodiment 112, wherein the emulsion adjuvant is an oil-in-water (o/w) emulsion adjuvant or a water-in-oil (w/o) emulsion adjuvant.
114. The broad-spectrum, pan-COV immunogenic composition of embodiment 113 or the broad-spectrum, pan-COV immunogenic composition of embodiment 113, wherein the oil-in-water (o/w) emulsion adjuvant is a Freund's complete adjuvant or a Freund's incomplete adjuvant.
115. The broad-spectrum, pan-COV immunogenic composition of embodiment 113 or the broad-spectrum, pan-COV immunogenic composition of embodiment 113, wherein the water-in-oil (w/o) emulsion adjuvant comprises a mineral oil and a surfactant or a squalene oil and a surfactant, or an oil and a sapogenin glycoside, or any combination thereof.
116. The broad-spectrum, pan-COV immunogenic composition of embodiment 112 or the broad-spectrum, pan-COV immunogenic composition of embodiment 112, wherein the PAMP adjuvant a lipopolysaccharide or extract thereof, a flagellin polypeptide and hinge region thereof, a nucleotide-based adjuvant, or N-acetylmuramyl-L-alanyl-D-isoglutamine.
117. The broad-spectrum, pan-COV immunogenic composition of embodiment 116 or the broad-spectrum, pan-COV immunogenic composition of embodiment 116, wherein the lipopolysaccharide or extract thereof is a monophosphoryl lipid A, a glucopyranosyl lipid adjuvant, or an aminoalkyl glucopyranoside.
118. The broad-spectrum, pan-COV immunogenic composition of embodiment 116 or the broad-spectrum, pan-COV immunogenic composition of embodiment 116, wherein the nucleotide-based adjuvant is a TLR7, a TLR8, or an oligonucleotide motif.
119. The broad-spectrum, pan-COV immunogenic composition of embodiment 118 or the broad-spectrum, pan-COV immunogenic composition of embodiment 118, wherein the oligonucleotide motif is a poly Inosine:Cytosine (poly I:C) motif or cytosine phosphoguanine (CpG) motif.
120. The broad-spectrum, pan-COV immunogenic composition of embodiment 112 or the broad-spectrum, pan-COV immunogenic composition of embodiment 112, wherein the molecular adjuvant is a cytokine, a chemokine, an immune costimulatory molecule, a toll-like receptor agonist, or an immune suppressive pathway inhibitor.
121. The broad-spectrum, pan-COV immunogenic composition of embodiment 111 or the broad-spectrum, pan-COV immunogenic composition of embodiment 111, wherein the one or more inorganic adjuvants comprise an aluminum-based adjuvant, a calcium salt, a colloidal iron hydroxide, a zirconium salt, or a zinc sulfate.
122. The broad-spectrum, pan-COV immunogenic composition of any one of embodiments 1-70 or the broad-spectrum, pan-COV immunogenic composition of any one of embodiments 71-108, further comprising one or more carriers.
123. The broad-spectrum, pan-COV immunogenic composition of embodiment 122 or the broad-spectrum, pan-COV immunogenic composition of embodiment 122, wherein the carrier is a vaccine delivery system (VDS).
124. The broad-spectrum, pan-COV immunogenic composition of embodiment 122 or the broad-spectrum, pan-COV immunogenic composition of embodiment 122, wherein the carrier is a vaccine adjuvant-delivery system (VADS).
125. The broad-spectrum, pan-COV immunogenic composition of any one of embodiments 122-124 or the broad-spectrum, pan-COV immunogenic composition of any one of embodiments 122-124 formulated for an oral route of administration, a dermal route of administration, or a parenteral route of administration.
126. The broad-spectrum, pan-COV immunogenic composition of any one of embodiments 122-125 or the broad-spectrum, pan-COV immunogenic composition of any one of embodiments 122-125, wherein the carrier is an emulsion carrier, a liposome carrier, a niosome carrier, a microsphere carrier, a nanoparticle carrier, or a carbon nanotube carrier.
127. The broad-spectrum, pan-COV immunogenic composition of embodiment 126 or the broad-spectrum, pan-COV immunogenic composition of embodiment 126, wherein the liposome carrier comprises (i) a mixture of anionic lipids; (ii) a mixture of cationic lipids; (iii) a mixture of zwitterionic lipids; (iv) a mixture of anionic lipids and cationic lipids; (v) a mixture of anionic lipids and zwitterionic lipids; (vi) a mixture of zwitterionic lipids and cationic lipids; or (vii) a mixture of anionic lipids, cationic lipids and zwitterionic lipids.
128. The broad-spectrum, pan-COV immunogenic composition of embodiment 126 or the broad-spectrum, pan-COV immunogenic composition of embodiment 126, wherein the liposome carrier comprises one or more cationic surfactants and/or an apolar fraction from a total lipid extract of a mycobacterium.
129. The broad-spectrum, pan-COV immunogenic composition of embodiment 126 or the broad-spectrum, pan-COV immunogenic composition of embodiment 126, wherein the nanoparticle carrier is an inorganic nanoparticle carrier, a polymeric nanoparticle carrier, or a biologically derived nanoparticle carrier.
130. The broad-spectrum, pan-COV immunogenic composition of embodiment 129 or the broad-spectrum, pan-COV immunogenic composition of embodiment 129, wherein the polymeric nanoparticle carrier comprises poly(D,L-lactide-co-glycolide) (PLGA), poly(lactic acid) (PLA), poly(γ-glutamic acid) (γ-PGA), poly(ethylene glycol) (PEG), poly(styrene), poly(N-vinylpyrrolidone), poly(ahydroxy acids), poly(hydroxy butyric acids), poly(lactones), including poly(caprolactones), poly(dioxanones), poly(valerolactone), poly(orthoesters), poly(anhydrides), poly(cyanoacrylates), tyrosine-derived poly(carbonates), poly(vinyl-pyrrolidinones) or poly(ester-amides), chitosan, dextran, or any combination thereof.
131. The broad-spectrum, pan-COV immunogenic composition of embodiment 129 or the broad-spectrum, pan-COV immunogenic composition of embodiment 129, wherein the biologically derived nanoparticle carrier is a lipid nanoparticle (LNP) carrier, a nanolipoprotein (NLP) carrier, a virus carrier, a virus-like particle (VLP) carrier, a virosome carrier, a synthetic virus like particle (SVLP) carrier, a self-assembling protein nanoparticle (SAPN) carrier, a carbon nanotube carrier, or a peptide carrier.
132. The broad-spectrum, pan-COV immunogenic composition of embodiment 131 or the broad-spectrum, pan-COV immunogenic composition of embodiment 131, wherein the lipid nanoparticle carrier comprises (i) a cationic lipid, (ii) a mixture of a cationic lipid and a sterol, (iii) a mixture of a cationic lipid and a neutral phospholipid, (iv) a mixture of a cationic lipid, sterol, and a PEGylated phospholipid, (v) a mixture of a cationic lipid, a neutral phospholipid, and a PEGylated phospholipid, (vi) a mixture of a cationic lipid, sterol, a PEGylated phospholipid, and a polymer, (vii) a mixture of a cationic lipid, a neutral phospholipid, a PEGylated phospholipid, and a polymer.
133. The broad-spectrum, pan-COV immunogenic composition of embodiment 131 or the broad-spectrum, pan-COV immunogenic composition of embodiment 131, wherein the virus-like particle carrier includes components from an icosahedral or spherical plant virus, components from a rod-shaped plant virus, or components from a bacteriophage.
134. The broad-spectrum, pan-COV immunogenic composition of embodiment 131 or the broad-spectrum, pan-COV immunogenic composition of embodiment 131, wherein the self-assembling protein nanoparticle carrier includes a caged protein nanoparticle carrier, a proteosome nanoparticle carrier, a cochleate nanoparticle carrier, or a dendrimer nanoparticle carrier.
135. The broad-spectrum, pan-COV immunogenic composition of embodiment 134 or the broad-spectrum, pan-COV immunogenic composition of embodiment 134, wherein the caged protein nanoparticle carrier is a ferritin nanoparticle carrier, an encapsulin nanoparticle carrier, a sulfur oxygenase reductase nanoparticle carrier, a lumazine synthase nanoparticle carrier, a small heat-shock proteins (sHSP) nanoparticle carrier, a dihydrolipoamide acetyltransferase (E2) nanoparticle carrier, or vault protein nanoparticle carrier.
136. The broad-spectrum, pan-COV immunogenic composition of any one of embodiments 1-70 or 109-135 or the broad-spectrum, pan-COV immunogenic composition of any one of embodiments 71-135, wherein the polypeptide is necessary to elicit, evoke or otherwise stimulate an immune response against one or more Coronavirus species, one or more Coronavirus strains, one or more Coronavirus genotypes, one or more Coronavirus subgenotypes, one or more Coronavirus serotypes or one or more Coronavirus genetic/antigenic lineages.
137. The broad-spectrum, pan-COV immunogenic composition of any one of embodiments 1-70 or 109-135 or the broad-spectrum, pan-COV immunogenic composition of any one of embodiments 71-135, wherein the polypeptide is necessary to elicit, evoke or otherwise stimulate an immune response against one or more Coronavirus species, one or more Coronavirus strains, one or more Coronavirus genotypes, one or more Coronavirus subgenotypes, one or more Coronavirus serotypes or one or more Coronavirus genetic/antigenic lineages to produce a cellular and/or humoral response capable of recognizing infected cells and inducing cytotoxicity and/or neutralizing antibodies against the one or more Coronavirus species, one or more Coronavirus strains, one or more Coronavirus genotypes, one or more Coronavirus subgenotypes, one or more Coronavirus serotypes or one or more Coronavirus genetic/antigenic lineages.
138. The broad-spectrum, pan-COV immunogenic composition of embodiment 136 or 137 or the broad-spectrum, pan-COV immunogenic composition of embodiment 136 or 137, wherein the one or more Coronavirus species, one or more Coronavirus strains, one or more Coronavirus genotypes, one or more Coronavirus subgenotypes, one or more Coronavirus serotypes or one or more Coronavirus genetic/antigenic lineages is from an Alphacoronavirus, a Betacoronavirus, a Gammacoronavirus, a Deltacoronavirus, or any combination thereof.
139. The broad-spectrum, pan-COV immunogenic composition of embodiment 138 or the broad-spectrum, pan-COV immunogenic composition of embodiment 138, wherein the Alphacoronavirus comprises Colacovirus, Decacovirus, Duvinacovirus, Luchacovirus, Minacovirus, Minunacovirus, Myotacovirus, Nyctacovirus, Pedacovirus, Rhinacovirus, Setracovirus, Soracovirus, Sunacovirus, Tegacovirus, or any combination thereof; wherein the Betacoronavirus comprises Embecovirus, Hibecovirus, Merbecovirus, Nobecovirus, Sarbecovirus, or any combination thereof; wherein the Gammacoronavirus comprises Brangacovirus, Cegacovirus, Igacovirus, or any combination thereof; and/or wherein the Alphacoronavirus comprises Andecovirus, Buldecovirus, Herdecovirus, or any combination thereof.
140. A method of treating a Coronavirinae-based infection or pathology by administering a broad-spectrum, pan-COV immunogenic composition as defined in embodiments 1-70 or 109-139 or the broad-spectrum, pan-COV immunogenic composition of any one of embodiments 71-139 to an individual.
141. A broad-spectrum, pan-COV immunogenic composition as defined in embodiments 1-70 or 109-139 or the broad-spectrum, pan-COV immunogenic composition of any one of embodiments 71-139 for use in treating a Coronavirinae-based infection or pathology.
142. A use of a broad-spectrum, pan-COV immunogenic composition as defined in embodiments 1-70 or 109-139 or the broad-spectrum, pan-COV immunogenic composition of any one of embodiments 71-139 in treating a Coronavirinae-based infection or pathology.
143. A use of a broad-spectrum, pan-COV immunogenic composition as defined in embodiments 1-70 or 109-139 or the broad-spectrum, pan-COV immunogenic composition of any one of embodiments 71-139 in the manufacture of a medicament for treating a Coronavirinae-based infection or pathology.
144. The method of embodiment 140 or the use of any one of embodiments 141-143, wherein the Coronavirinae-based infection or pathology is caused by one or more Coronavirus species, one or more Coronavirus strains, one or more Coronavirus genotypes, one or more Coronavirus subgenotypes, one or more Coronavirus serotypes or one or more Coronavirus genetic/antigenic lineages, or any combination thereof.
145. A method of creating, maintaining or restoring antigenic memory to one or more Coronavirus species, one or more Coronavirus strains, one or more Coronavirus genotypes, one or more Coronavirus subgenotypes, one or more Coronavirus serotypes or one or more Coronavirus genetic/antigenic lineages in an individual or population of individuals comprises administering a broad-spectrum, pan-COV immunogenic composition as defined in embodiments 1-70 or 109-139 or the broad-spectrum, pan-COV immunogenic composition of any one of embodiments 71-139 to an individual.
146. A broad-spectrum, pan-COV immunogenic composition as defined in embodiments 1-70 or 109-139 or the broad-spectrum, pan-COV immunogenic composition of any one of embodiments 71-139 for use in creating, maintaining or restoring antigenic memory to one or more Coronavirus species, one or more Coronavirus strains, one or more Coronavirus genotypes, one or more Coronavirus subgenotypes, one or more Coronavirus serotypes or one or more Coronavirus genetic/antigenic lineages in an individual or population of individuals.
147. A use of a broad-spectrum, pan-COV immunogenic composition as defined in embodiments 1-70 or 109-139 or the broad-spectrum, pan-COV immunogenic composition of any one of embodiments 71-139 in creating, maintaining or restoring antigenic memory to one or more Coronavirus species, one or more Coronavirus strains, one or more Coronavirus genotypes, one or more Coronavirus subgenotypes, one or more Coronavirus serotypes or one or more Coronavirus genetic/antigenic lineages in an individual or population of individuals.
148. A use of a broad-spectrum, pan-COV immunogenic composition as defined in embodiments 1-70 or 109-139 or the broad-spectrum, pan-COV immunogenic composition of any one of embodiments 71-139 in the manufacture of a medicament for creating, maintaining or restoring antigenic memory to one or more Coronavirus species, one or more Coronavirus strains, one or more Coronavirus genotypes, one or more Coronavirus subgenotypes, one or more Coronavirus serotypes or one or more Coronavirus genetic/antigenic lineages in an individual or population of individuals.
149. The method of any one of embodiments 140, 144, or 145 or the use of any one of embodiments 141-144 or 146-148, wherein broad-spectrum, pan-COV immunogenic composition or the broad-spectrum, pan-COV immunogenic composition elicits, evokes or otherwise stimulates an immune response against one or more Coronavirus species, one or more Coronavirus strains, one or more Coronavirus genotypes, one or more Coronavirus subgenotypes, one or more Coronavirus serotypes or one or more Coronavirus genetic/antigenic lineages.
150. The method of any one of embodiments 140, 144, or 145 or the use of any one of embodiments 141-144 or 146-148, wherein broad-spectrum, pan-COV immunogenic composition or the broad-spectrum, pan-COV immunogenic composition elicits, evokes or otherwise stimulates an immune response against one or more Coronavirus species, one or more Coronavirus strains, one or more Coronavirus genotypes, one or more Coronavirus subgenotypes, one or more Coronavirus serotypes or one or more Coronavirus genetic/antigenic lineages to produce a cellular and/or humoral response capable of recognizing infected cells and inducing cytotoxicity and/or neutralizing antibodies against the one or more Coronavirus species, one or more Coronavirus strains, one or more Coronavirus genotypes, one or more Coronavirus subgenotypes, one or more Coronavirus serotypes or one or more Coronavirus genetic/antigenic lineages.
151. The method of any one of embodiments 144, 145, 149, or 150 or use of any one of embodiments 146-150, wherein the one or more Coronavirus species, one or more Coronavirus strains, one or more Coronavirus genotypes, one or more Coronavirus subgenotypes, one or more Coronavirus serotypes or one or more Coronavirus genetic/antigenic lineages is from an Alphacoronavirus, a Betacoronavirus, a Gammacoronavirus, a Deltacoronavirus, or any combination thereof.
152. The method of embodiment 151 or use of embodiment 151, wherein the Alphacoronavirus comprises Colacovirus, Decacovirus, Duvinacovirus, Luchacovirus, Minacovirus, Minunacovirus, Myotacovirus, Nyctacovirus, Pedacovirus, Rhinacovirus, Setracovirus, Soracovirus, Sunacovirus, Tegacovirus, or any combination thereof; wherein the Betacoronavirus comprises Embecovirus, Hibecovirus, Merbecovirus, Nobecovirus, Sarbecovirus, or any combination thereof; wherein the Gammacoronavirus comprises Brangacovirus, Cegacovirus, Igacovirus, or any combination thereof; and/or wherein the Alphacoronavirus comprises Andecovirus, Buldecovirus, Herdecovirus, or any combination thereof.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the anitgens, imminogenic compositions, or methods and uses disclosed herein.

### Example 1

### Identification of COV Antigenic Sequences

A computer algorithm capable of predicting T cell reactive epitopes within protein sequences peptides was used to identify Coronavirus antigens. In this in-silico model as many protein sequences as possible and from as many different strains were gathered from Coronavirus pathogens. Available sequences for a particular protein were then aligned using Clustal and a consensus sequence was generated using Jalview. This sequence was then uploaded into the algorithm which predicted all possible T cell reactive epitopes. Peptide sequences from regions containing high number of epitopes were identified and these highly T-cell immunogenic regions were further analysed for conservation amongst the different strains. Conserved peptide sequences of between 20-40 amino acids in length in which consecutive amino acids share at least 70% sequence identity amongst all the other sequences found were selected and further analyzed. For example, in order to avoid inducing autoimmune reactions post-vaccination, these conserved peptide sequences were evaluated to confirm that these sequence did not share significant identity with other human and murine protein sequences. In addition, these conserved peptide sequences were evaluated to confirm these sequence could be synthetically manufactured by F-moc chemistry.

Using this approach, a total of 12 peptides from two different proteins were identified (Table 1). SEQ ID NO: 1 (RdRp-α) is a 27 amino acid peptide present in the RNA dependent RNA polymerase from alphacoronaviruses. SEQ ID NO: 2 (RdRp-βγ) is a 27 amino acid peptide present in the RNA dependent RNA polymerase from both betacoronaviruses and gammacoronaviruses. SEQ ID NO: 3 (RdRp-βδ) is a 27 amino acid peptide present in the RNA dependent RNA polymerase from both betacoronaviruses and deltacoronaviruses. SEQ ID NO: 4 (Mα) is a 19 amino acid peptide present in the M protein from alphacoronaviruses. SEQ ID NO: 5 (Mα) is a 19 amino acid peptide present in the M protein from alphacoronaviruses. SEQ ID NO: 6 (Mα) is a 19 amino acid peptide present in the M protein from alphacoronaviruses. SEQ ID NO: 7 (Mα) is a 19 amino acid peptide present in the M protein from alphacoronaviruses. SEQ ID NO: 8 (Mα) is a 19 amino acid peptide present in the M protein from alphacoronaviruses. SEQ ID NO: 9 (Mβ) is a 23 amino acid peptide present in the M protein from betacoronaviruses. SEQ ID NO: 10 (Mβ) is a 23 amino acid peptide present in the M protein from betacoronaviruses. SEQ ID NO: 11 (Mγ) is a 24 amino acid peptide present in the M protein from gammacoronavirus. SEQ ID NO: 12 (Mδ) is a 18 amino acid peptide present in the M protein from a deltacoronavi ruses.

### Example 2

### Peptide Manufacturing

Peptides will be manufactured by conventional Fmoc chemistry (Bachem AG, Switzerland) via automated Solid Phase Peptide Synthesis (SPPS) using a peptide synthesizer in accordance with Good Manufacturing Practice (GMP). The initial amino acid derivative is loaded on solid resin (2-Chlorotrityl chloride resin) under alkaline conditions and subsequent amino acid derivatives are coupled by Fmoc-strategy as alternating coupling and de-protecting steps. Cleavage of the protected peptides from the resin and removal of the sidechain protecting groups. The resulting peptides obtained as TFA salts are purified by preparative HPLC chromatography, followed by conversion into the acetate salts. The final products are lyophilized and stored for future use.

For the formulation of the final vaccine, the individual peptides are reconstituted with an appropriate solvent and mixed together in an equimolar manner. For administration to animals or humans, and adjuvant may be added to the mix of peptides.

### Example 3

### Nucleotide Manufacturing

A nucleotide sequence (e.g., a DNA or RNA) encoding a polypeptide of of Polypeptide Structure 1-IV will be manufactured by conventional recombinant and molecular biology techniques.

For a DNA sequence, a completely functional gene comprising 1) the open-reading frame for the amino acid sequence of the encoded polypeptide; 2) 5′- and 3′-UTR sequences, and 3) strong polyadenylation/transcriptional termination signal, will be codon-optimised for human expression and a synthetic DNA molecule produced using standard gene synthesis procedures. The synthetic DNA gene will be subcloned into a plasmid DNA expression construct comprising a strong viral promotor to drive the in vivo transcription and translation of the gene. This expression construct will be purified, analysed for integrity, and stored for future use.

For a mRNA sequence, a completely functional mRNA sequence comprising 1) the open-reading frame for the amino acid sequence of the encoded polypeptide and 2) 5′- and 3′-UTR sequences will be codon-optimised for human expression and a synthetic DNA molecule produced using standard gene synthesis procedures. The synthetic DNA sequences will be subcloned into a plasmid DNA construct comprising an RNA polymerase promoter, which after sequencing and purification, is used to generate a Tail-PCR product having a poly(A) tail structure. This product will be purified and analysed by for example capillary electrophoresis, followed by use of the purified product as PCR template in in vitro transcription test reactions to produce RNA. The PCR product will be purified and analysed for integrity by capillary electrophoresis, and then will be used as a template in production scale IVT reaction with NTP-mix including 100% m5C, 100% pseudo-U. Following DNase treatment to eliminate any left-over DNA template, the mRNA is purified and analysed for integrity by capillary electrophoresis. After enzyme capping, the mRNA is purified, analysed for integrity by capillary electrophoresis, and stored for future use.

To analyse the expression of an encoded polypeptide from a DNA or mRNA, adherent cells, such as HeLa cells, will be grown to an appropriate density, e.g., 70-90% confluency and the mRNA will be introduced into cells by, e.g., transfection or transduction. A proteasome inhibitor may be added at the time of the transfection to prevent degradation of the translated protein encoded in the mRNA. After about 48 hours of incubation, the supernatant will be collected and the cell monolayers will be lysed using lysis buffer containing a proteinase inhibitor cocktail. Detection of the translated protein will be ascertained by separating the protein fractions in the cell supernatants and/or cell lysates by SDS-PAGE, transferring the protein from the gel to a membrane and then incubating it with antibodies specific for the protein or for a tag included in the protein such as a 6×His tag or HA tag. These antibodies or additional detection antibodies are labelled with HRP or AP. An enzymatic reaction will allow visualization of the band where the protein of interest is either by generation of colour or luminescence. Other ways of detecting the protein in intact cells would include cell based ELISAs or flow cytometry.

For the formulation of the final vaccine, the DNA or RNA molecules are reconstituted with an appropriate solvent. For administration to animals or humans, and adjuvant may be added to the DNA or RNA molecules. In order to protect the DNA or RNA from degradation, they may formulated in LNPs, NLPs or included in a virus.

### Example 4

### Recombinant Protein Manufacturing

A recombinant polypeptide of Polypeptide Structures 1-IV will be manufactured by conventional recombinant and molecular biology techniques.

A completely functional gene comprising 1) the open-reading frame for the amino acid sequence of the encoded polypeptide; 2) 5′- and 3′-UTR sequences, and 3) strong polyadenylation/transcriptional termination signal, will be codon-optimised for expression in the chosen expression organism and a synthetic DNA molecule produced using standard gene synthesis procedures. The synthetic DNA gene will be subcloned into an expression construct comprising a strong viral promotor to drive the in vivo transcription and translation of the gene. This expression construct will be purified, analysed for integrity, and stored for future use. For production of the recombinant protein, an expression construct will be introduced into host cells by, e.g., transfection or transduction, and the subsequently expressed polypeptide will be isolated and examined for composition integrity and/or activity.

For the formulation of the final vaccine, the recombinant protein is reconstituted with an appropriate solvent. For administration to animals or humans, and adjuvant may be added to the recombinant protein.

### Example 5

### In vivo Immunogenicity Testing of COV Immunogenic Compositions

For peptide vaccine testing, individual peptides produced in Example 2, are reconstituted with an appropriate solvent and a solution is prepared by adding peptide in equimolar amounts of between 0.5 nmol and 2.0 nmol and mixed together. The final volume of the peptide vaccine administered will depend on the route of administration. For a subcutaneous dose in mice the volume of vaccine administer will be 0.2 mL.

For DNA or RNA vaccine testing, mRNA produced in Example 3 are encapsulated in lipid nanoparticles (LNPs) or nano lipoproteins (NLPs), with a RALA destabilizing peptide. The final amount of the RNA included in the vaccine will be between 5 μg to 5 μg, this amount will be adjusted empirically. Encapsulation efficiency will be measured using a Ribogreen assay and particle stability will be assessed by measuring particle size, zeta potential and encapsulation efficiency over time. The final volume of the RNA vaccine administered will depend on the route of administration. For intramuscular or intradermal administration in mice the volume of vaccine administer will be 0.05 mL.

For recombinant protein vaccine testing, recombinant protein produced in Example 4, is reconstituted with an appropriate solvent and a solution is prepared by adding recombinant protein in an amount of between 0.5 nmol and 2.0 nmol. The final volume of the recombinant protein vaccine administered will depend on the route of administration. For a subcutaneous dose in mice the volume of vaccine administer will be 0.2 mL while for an intradermal or intramuscular dose in mice the volume of vaccine administer will be 0.050 mL.

To determine humoral and cellular responses, a C57/BLK/6 mice model will be used. C57/BLK/6 mice barrier bred (i.e. pathogen free), 7-10 weeks old at the start of the study will be divided into three groups with each group having 5 animals. Group A mice will be administered one dose of the active treatment (i.e., a COV peptide antigen mix, or a COV recombinant protein, or a COV mRNA or a COV DNA construct); Group B mice will be administered two doses of the active treatment with the second dose occurring 14-21 days after the first; and Group C mice will be administered a vehicle control. Animals will be culled 14-21 days after the last dose and spleens and terminal blood will be harvested for analysis. Other blood collection points could include pre-vaccination or between doses.

Additionally, follow-on studies to the ones described above could also be performed, including testing different amounts of peptide, DNA, mRNA or recombinant protein included in the vaccine, testing different combinations of individual peptides included in a vaccine, testing the inclusion of multiple DNA, mRNA, or recombinant protein included in a vaccine, testing the inclusion of an adjuvant, testing different encapsulation methods used to produce the DNA and mRNA vaccines, testing different number of doses administered into the animals, or testing different delivery routes.

Vaccine-specific antibodies will be measured by assessing total Ig titers specific in the sera or plasma from the blood samples of the animals in all groups. ELISA 96-well plates will be coated overnight at +4° C. with 2 μM of single or pooled Coronovirus peptides. Plates will be washed twice with PBST (PBS with 0.05% TWEEN 20) and blocked for 1 hour with 1% BSA Fraction V in PBS. Plates will be washed thrice with PBST before adding test sera samples at double decreasing dilutions from 1:50 to 1:3, 200 and incubating for 2 hours. After washing six times with PBST, wells will be loaded with HRP-conjugated anti-mouse-Ig sera. After 1 hour incubation, plates will be washed eight times with PBST, and TMB substrate will be added. The reaction will be stopped with 0.5 M H2504 and the absorbance of each well read at 450 nm. It is expected that the results will show a better antibody response in the vaccinated animals versus the unvaccinated controls.

T cell responses will be assessed by cytokine ELISA. Mouse spleens will be gently pressed through cell strainers and red blood cells will be removed with red cell lysis buffer (nine parts 0.16 M NH4C1 and one part of 0.17 M Tris, pH 7.2). Splenocyte suspensions from each experimental group will be plated in 96-well plates at a density of 4×10⁶ cells/well in RPMI-1640 supplemented with 50 IU/50 μg/mL of penicillin/streptomycin and 10% FCS, and containing either media alone, Concanavalin A (Con A)(5 μg/mL), single or pooled polyepitope COV peptides at 0.5 μM to 2 μM each or 15-mer overlapping peptides covering the length of the COV peptides. After 24-48 hours of incubation at 37° C. and 5% CO₂, the supernatant will be collected and analysed for the presence of IFN-γ and IL-4 production using a mouse cytokine ELISA kit (BD Biosciences) to assess the induction of an immune response. Responses will be plotted as the differential in cytokine production (μg/mL) between the groups immunised with the vaccine and their respective controls. Animals in all groups are expected to react to Con A with IFN-γ levels above 1000 μg/mL and IL-4 levels above 200 μg/mL. It is expected that no animals will show spontaneous activation, therefore all media alone readings should be below or around the threshold of detection for both IFN-γ and IL-4. It is expected that little or no IL-4 will be secreted in response to the various vaccine antigens compared to the secretion observed with Con A non-specific stimulation of the splenocytes. It is also expected that vaccinated animals will be secreting higher amounts of IFN-γ than unvaccinated animal controls when cells are cultured with vaccine-specific antigens.

T cell responses will be assessed by flow cytometry. Mouse spleens will be gently pressed through cell strainers and red blood cells will be removed with red cell lysis buffer (nine parts 0.16 M NH4C1 and one part of 0.17 M Tris, pH 7.2). Splenocyte suspensions from each experimental group will be plated in 96-well plates at a density of 4×10⁶ cells/well in RPMI-1640 supplemented with 50 IU/50 μg/mL of penicillin/streptomycin and 10% FCS. Splenocytes will be incubated at 37° C. and 5% CO₂ for 6 hours to 24 hours with media alone, Concanavalin A (Con A)(5 μg/mL) or another non-specific stimulant such as PMA or LPS, single or pooled polyepitope COV peptides at 0.5 μM to 2 μM each or 15-mer overlapping peptides covering the length of the COV peptides. After incubation, cells will be stained with different antibodies for example extracellular staining with anti-CD3, anti-CD4, anti-CD8 to determine the cell type (CD3+CD4+ are T-helper cells, and CD3+CD8+ are cytotoxic T cells) and intracellular staining with anti-IFN-γ, anti-TNFα, anti-IL-2 to detect cells producing these cytokines which are indicative of a Th1 stimulation of these cells. The antibodies used are labelled with fluorescence and with the use of a flow cytometer, the amount of fluorescence for each antibody used can be measured in every single cell. It is expected that the splenocytes stimulated with non-specific stimulant will show high staining for CD3, CD3 and CD8, and IFNgamma, TNF and IL-2 as these non-specific stimulants can induce cell proliferation and production of these cytokines. In contrast, splenocytes cultured with media only should show lower levels of these markers and will be used to determine the background levels. Splenocytes from animals vaccinated with COV antigens should result in a higher number of cells positively stained for CD3, CD4 and CD8 and the cytokines IFN-γ, TNFα, and IL-2.

T cell responses will be assessed by ELISPOT. Mouse spleens will be gently pressed through cell strainers and red blood cells will be removed with red cell lysis buffer (nine parts 0.16 M NH4C1 and one part of 0.17 M Tris, pH 7.2). Splenocyte suspensions from each experimental group will be plated in 96-well plates, coated with anti-IFN-γ or another antibodies specific for the soluble marker of interest, at a density of 4×10⁶ cells/well in RPMI-1640 supplemented with 50 IU/50 μg/mL of penicillin/streptomycin and 10% FCS. Splenocyte are then stimulated with peptides in the vaccine, or overlapping peptides corresponding to the polypeptide encoded by the DNA or RNA vaccine or recombinant protein, and incubated at 37° C. and 5% CO₂ for 6h to 24 h. Plates will be washed and analysed for the presence of IFN-γ or other soluble marker of interest using a color detection system and an ELISpot reader to identify splenocyte producing IFN-γ or other soluble marker of interest. It is expected that vaccinated animals will be secreting higher amounts of IFN-γ than unvaccinated animal controls in the wells where vaccine antigens were added compared to wells with media only.

### Example 6

### Efficacy Testing of COV Immunogenic Compositions

Once the optimal dose/formulation has been optimised for immune purposes, it will be tested in efficacy studies in an in vivo animal model for the disease or in an in vitro surrogate assay if animal models are not available. Currently there are three viruses within the Coronavirus family that can be tested in animals. SARS-cov-1 and SARS-cov-2 can be tested in hamsters, and efficacy against MERS can be tested in rabbits. Since immunogenicity is tested in mice but efficacy is tested in hamsters and/or rabbits, a bridging immunogenicity study will be performed prior to the efficacy studies to ensure that the candidate vaccines generate similar immune responses in hamsters and rabbits than in mice using an equivalent dose. Each challenge study will include two groups, a vaccinated group with n=6 and a placebo group with n=6. Animals will be vaccinated with the optimised dosing regime and 14 to 21 dayas later will be challenged with the virus. Animals will; be assessed daily for body weight loss, virology and pathology. It is expected that animals vaccinated with the COV antigens will experience fewer signs of disease than animals in the placebo group.

Sometimes the animal model of disease may not be the same as the species where immunogenicity was tested, and a bridging study may be needed where immunogenicity is tested in the species where efficacy will be tested to ensure that a similar immune response is observed.

In closing, foregoing descriptions of embodiments of the present invention have been presented for the purposes of illustration and description. It is to be understood that, although aspects of the present invention are highlighted by referring to specific embodiments, one skilled in the art will readily appreciate that these described embodiments are only illustrative of the principles comprising the present invention. As such, the specific embodiments are not intended to be exhaustive or to limit the invention to the precise forms disclosed. Therefore, it should be understood that embodiments of the disclosed subject matter are in no way limited to a particular element, compound, composition, component, article, apparatus, methodology, use, protocol, step, and/or limitation described herein, unless expressly stated as such.

In addition, groupings of alternative embodiments, elements, steps and/or limitations of the present invention are not to be construed as limitations. Each such grouping may be referred to and claimed individually or in any combination with other groupings disclosed herein. It is anticipated that one or more alternative embodiments, elements, steps and/or limitations of a grouping may be included in, or deleted from, the grouping for reasons of convenience and/or patentability. When any such inclusion or deletion occurs, the specification is deemed to contain the grouping as modified, thus fulfilling the written description of all Markush groups used in the appended claims.

Furthermore, those of ordinary skill in the art will recognize that certain changes, modifications, permutations, alterations, additions, subtractions and sub-combinations thereof can be made in accordance with the teachings herein without departing from the spirit of the present invention. Furthermore, it is intended that the following appended claims and claims hereafter introduced are interpreted to include all such changes, modifications, permutations, alterations, additions, subtractions and sub-combinations as are within their true spirit and scope. Accordingly, the scope of the present invention is not to be limited to that precisely as shown and described by this specification.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

The words, language, and terminology used in this specification is for the purpose of describing particular embodiments, elements, steps and/or limitations only and is not intended to limit the scope of the present invention, which is defined solely by the claims. In addition, such words, language, and terminology are to be understood not only in the sense of their commonly defined meanings, but to include by special definition in this specification structure, material or acts beyond the scope of the commonly defined meanings. Thus, if an element, step or limitation can be understood in the context of this specification as including more than one meaning, then its use in a claim must be understood as being generic to all possible meanings supported by the specification and by the word itself.

The definitions and meanings of the elements, steps or limitations recited in a claim set forth below are, therefore, defined in this specification to include not only the combination of elements, steps or limitations which are literally set forth, but all equivalent structure, material or acts for performing substantially the same function in substantially the same way to obtain substantially the same result. In this sense it is therefore contemplated that an equivalent substitution of two or more elements, steps and/or limitations may be made for any one of the elements, steps or limitations in a claim set forth below or that a single element, step or limitation may be substituted for two or more elements, steps and/or limitations in such a claim. Although elements, steps or limitations may be described above as acting in certain combinations and even initially claimed as such, it is to be expressly understood that one or more elements, steps and/or limitations from a claimed combination can in some cases be excised from the combination and that the claimed combination may be directed to a sub-combination or variation of a sub-combination. As such, notwithstanding the fact that the elements, steps and/or limitations of a claim are set forth below in a certain combination, it must be expressly understood that the invention includes other combinations of fewer, more or different elements, steps and/or limitations, which are disclosed in above combination even when not initially claimed in such combinations. Furthermore, insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. Accordingly, the claims are thus to be understood to include what is specifically illustrated and described above, what is conceptually equivalent, what can be obviously substituted and also what essentially incorporates the essential idea of the invention.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as, e.g., “first,” “second,” “third,” etc. —for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising”, variations thereof such as, e.g., “comprise” and “comprises”, and equivalent open-ended transitional phrases thereof like “including”, “containing” and “having”, encompass all the expressly recited elements, limitations, steps, integers, and/or features alone or in combination with unrecited subject matter; the named elements, limitations, steps, integers, and/or features are essential, but other unnamed elements, limitations, steps, integers, and/or features may be added and still form a construct within the scope of the claim. Specific embodiments disclosed herein may be further limited in the claims using the closed-ended transitional phrases “consisting of” or “consisting essentially of” (or variations thereof such as, e.g., “consist of”, “consists of”, “consist essentially of”, and “consists essentially of”) in lieu of or as an amendment for “comprising.” When used in the claims, whether as filed or added per amendment, the closed-ended transitional phrase “consisting of” excludes any element, limitation, step, integer, or feature not expressly recited in the claims. The closed-ended transitional phrase “consisting essentially of” limits the scope of a claim to the expressly recited elements, limitations, steps, integers, and/or features and any other elements, limitations, steps, integers, and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps, integers, and/or features specifically recited in the claim, whereas the meaning of the closed-ended transitional phrase “consisting essentially of” is being defined as only including those elements, limitations, steps, integers, and/or features specifically recited in the claim and those elements, limitations, steps, integers, and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such, the embodiments described herein or so claimed with the phrase “comprising” expressly and unambiguously provide description, enablement, and support for the phrases “consisting essentially of” and “consisting of.”

Lastly, all patents, patent publications, and other references cited and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard is or should be construed as an admission that the inventors are not entitled to antedate such disclosure by virtue of prior invention or for any other reason. All statements as to the date or representation as to the contents of these documents are based on the information available to the applicant and do not constitute any admission as to the correctness of the dates or contents of these documents.

1. A broad-spectrum, pan-COV immunogenic composition, wherein
a) the broad-spectrum, pan-COV immunogenic composition comprises a nucleotide sequence comprising an open reading frame encoding a polypeptide of Polypeptide Structure I, or
b) the broad-spectrum, pan-COV immunogenic composition comprises a polypeptide of Polypeptide Structure I:
wherein polypeptide of Polypeptide Structure I is:
COVPA¹-(PLS¹ₘ)-[COVPA²-(PLS²ₘ)]ₙ-COVPA³ Polypeptide Structure I
wherein COVPA¹ is a first COV peptide antigen selected from a COV peptide antigen having an amino acid sequence of at least 75% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12,
COVPA² is a second COV peptide antigen selected from a COV peptide antigen having an amino acid sequence of at least 75% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12,
COVPA³ is a third COV peptide antigen selected from a COV peptide antigen having an amino acid sequence of at least 75% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12,
PLS¹ and PLS² are each independently a peptide linker/spacer,
m is independently any integer from 0 to 1,
n is any integer from 0 to 52,
wherein the polypeptide comprises at least two peptide antigens selected from COVPA1, COVPA2, COVPA3, or any combination thereof,
wherein for each n, COVPA² can be a different peptide antigen or the same peptide antigen, and
wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other.

2. The broad-spectrum, pan-COV immunogenic composition of claim 1, wherein COVPA¹, COVPA², and COVPA³ each have a different peptide antigen from each other.

3. The broad-spectrum, pan-COV immunogenic composition of claim 1,
wherein PLS¹ has an amino acid sequence of SEQ ID NO: 18, SEQ ID NO: 19, SEQ ID NO: 20, or SEQ ID NO: 21, and/or
wherein PLS² has an amino acid sequence of SEQ ID NO: 18, SEQ ID NO: 19, SEQ ID NO: 20, or SEQ ID NO: 21.

4. The broad-spectrum, pan-COV immunogenic composition of claim 1,
wherein n is any integer from 2 to 48, 4 to 46, 6 to 44, 8 to 42, 10 to 40, 12 to 38, 14 to 36, 16 to 34, 18 to 32, or 20 to 30, or
wherein n is any integer from 0 to 10, 0 to 20, 0 to 30, 0 to 40, 0 to 50, 5 to 10, 5 to 20, 5 to 30, 5 to 40, 5 to 50, 10 to 20, 10 to 30, 10 to 40, 10 to 50, or
wherein n is any integer from 0 to 4, 4, to 8, 8 to 12, 12 to 16, 16 to 20, 20 to 24, 24 to 28, 28 to 32, 32 to 36, 36 to 40, 40 to 44, 44 to 48, or 48 to 52.

5. The broad-spectrum, pan-COV immunogenic composition of claim 1, wherein the nucleotide sequence further comprises an open reading frame for a stabilizing peptide operably linked to and in frame with the open reading frame encoding the polypeptide.

6. The broad-spectrum, pan-COV immunogenic composition of claim 5, wherein the stabilizing peptide is a lysosomal-associated membrane protein (LAMP) or fragment thereof.

7. The broad-spectrum, pan-COV immunogenic composition of claim 1, wherein the nucleotide-sequence is a DNA nucleotide sequence, a RNA nucleotide sequence, or a self-amplifying RNA nucleotide sequence, or wherein the polypeptide is recombinantly produced.

8. A broad-spectrum, pan-COV immunogenic composition comprising one or more COV peptide antigens and an adjuvant, the one or more COV peptide antigens having a length of about 10 amino acids to about 60 amino acids and comprising an amino acid sequence of at least 75% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, or a fragment thereof.

9. The broad-spectrum, pan-COV immunogenic composition of claim 8, wherein the one or more COV peptide antigens are the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, or a fragment thereof.

10. The broad-spectrum, pan-COV immunogenic composition of claim 8, wherein the adjuvant comprises an organic adjuvant, an inorganic adjuvant, or combinations thereof.

11. A broad-spectrum, pan-COV immunogenic composition, wherein
a) the broad-spectrum, pan-COV immunogenic composition comprises a nucleotide sequence comprising an open reading frame encoding a polypeptide of Polypeptide Structure IV, or
b) the broad-spectrum, pan-COV immunogenic composition comprises a polypeptide of Polypeptide Structure IV:
wherein polypeptide of Polypeptide Structure IV is:
PA¹-(SCP¹)[PA²-(SCP²)]ₒ-PA³ Polypeptide Structure IV
wherein PA¹, PA², and PA³ each independently comprise a nucleotide sequence comprising an open reading frame encoding a polypeptide of Polypeptide Structure I:
COVPA¹-(PLS¹ₘ)-[COVPA²-(PLS²ₘ)]ₙ-COVPA³ Polypeptide Structure I
wherein COVPA¹ is a first COV peptide antigen selected from a COV peptide antigen having an amino acid sequence of at least 75% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12,
COVPA² is a second COV peptide antigen selected from a COV peptide antigen having an amino acid sequence of at least 75% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12,
COVPA³ is a third COV peptide antigen selected from a COV peptide antigen having an amino acid sequence of at least 75% identity to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12,
PLS¹ and PLS² are each independently a peptide linker/spacer,
m is independently any integer from 0 to 1,
n is any integer from 0 to 52,
wherein the polypeptide comprises at least two peptide antigens selected from COVPA1, COVPA2, COVPA3, or any combination thereof,
wherein for each n, COVPA² can be a different peptide antigen or the same peptide antigen, and
wherein each of COVPA¹, COVPA², and COVPA³ can be the same peptide antigen, each of COVPA¹, COVPA², and COVPA³ can be a different peptide antigen from each other, or COVPA¹, COVPA², and COVPA³ can comprises a peptide antigen subset of the same peptide antigen and a peptide antigen subset of peptide antigens that are different from each other,
wherein SCP¹ and SCP² are each independently a self-cleaving peptide, and
wherein o from Polypeptide Structure IV is any integer from 0 to 10.

12. The broad-spectrum, pan-COV immunogenic composition of claim 11, wherein PA¹, PA², and PA³ can each independently encode the same polypeptide or a different polypeptide from each other.

13. The broad-spectrum, pan-COV immunogenic composition of claim 11, wherein an internal ribosome entry site is independently operationally linked to the 5′ end of the nucleotide sequence encoding each PA², to the 5′ end of the nucleotide sequence encoding PA³, or to any combination thereof.

14. The broad-spectrum, pan-COV immunogenic composition of claim 11,
wherein SOP¹ has an amino acid sequence of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30, and/or
wherein SCP² has an amino acid sequence of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30.

15. The broad-spectrum, pan-COV immunogenic composition of claim 11, wherein COVPA¹, COVPA², and COVPA³ each have a different peptide antigen from each other.

16. The broad-spectrum, pan-COV immunogenic composition of claim 11,
wherein PLS¹ has an amino acid sequence of SEQ ID NO: 18, SEQ ID NO: 19, SEQ ID NO: 20, or SEQ ID NO: 21, and/or
wherein PLS² has an amino acid sequence of SEQ ID NO: 18, SEQ ID NO: 19, SEQ ID NO: 20, or SEQ ID NO: 21.

17. The broad-spectrum, pan-COV immunogenic composition of claim 11, wherein the nucleotide sequence further comprises an open reading frame for a stabilizing peptide operably linked to and in frame with the open reading frame encoding the polypeptide.

18. The broad-spectrum, pan-COV immunogenic composition of claim 17, wherein the stabilizing peptide is a lysosomal-associated membrane protein (LAMP) or fragment thereof.

19. The broad-spectrum, pan-COV immunogenic composition of claim 11, wherein the nucleotide-sequence is a DNA nucleotide sequence, a RNA nucleotide sequence, or a self-amplifying RNA nucleotide sequence, or wherein the polypeptide is recombinantly produced.
